(19) United States
(12) Patent Application Publication
(10) Pub. No.: US 2017/0216277 A1
(43) Pub. Date: Aug. 3, 2017

(54) THERAPEUTIC USE OF NALBUPHINE WITHOUT AQUARETIC EFFECTS

(71) Applicant: TREVI THERAPEUTICS, INC., New Haven, CT (US)

(72) Inventor: THOMAS SCIASCIA, BELMONT, MA (US)

(21) Appl. No.: 15/400,486

(22) Filed: Jan. 6, 2017

Related U.S. Application Data
(60) Provisional application No. 62/275,394, filed on Jan. 6, 2016.

(51) Int. Cl. A61K 31/485

(52) U.S. Cl. CPC A61K 31/485

(57) ABSTRACT

The present invention relates to methods for treating pruritus with anti-pruritic compositions, wherein the method provides a therapeutic effect without producing a substantial aquaretic effect.

### CROSS-REFERENCE TO RELATED APPLICATIONS

The present application claims the benefit of priority to U.S. Provisional Application No. 62/275,394, filed on Jan. 6, 2016, the contents of which is hereby incorporated by reference in its entirety.

### FIELD OF THE INVENTION

The present invention relates to methods for treating pruritus using nalbuphine compositions, wherein the method provides a therapeutic effect without producing a substantial aquaretic effect.

### BACKGROUND

Pruritus, or itch, is a sensation that stimulates the desire or reflex to scratch, which can be either generalized or localized. The cause of pruritus is not fully understood. Proposed contributors to the pathogenesis of pruritus may include anemia or other manifestation of erythropoietin deficiency, histamine release from skin mast cells, skin dryness, secondary hyperparathyroidism, hyperphosphatemia with increased calcium phosphate deposition in the skin and alterations in the endogenous opioidergic system with overexpression of opioid μ-receptors.

### SUMMARY OF THE INVENTION

The present invention, among other things, provides methods of treating pruritus comprising administering an effective amount of an anti-pruritus agent to a subject in need of such treatment, wherein the method provides a therapeutic effect without producing a substantial aquaretic effect. In one embodiment, the anti-pruritic agent is nalbuphine or a pharmaceutically acceptable salt, solvate or ester thereof.

In one embodiment, the present invention provides methods for treating various pruritic conditions using nalbuphine or a pharmaceutically acceptable salt, solvate or ester thereof, wherein the administration of nalbuphine or a pharmaceutically acceptable salt, solvate or ester thereof provides a therapeutic effect without producing a substantial aquaretic effect.

In one embodiment, the method of treating pruritus comprising administering an effective amount of an anti-pruritic agent such as nalbuphine or a pharmaceutically acceptable salt, solvate or ester thereof to a subject does not significantly alter the total daily urine volume in the subject upon administration of the anti-pruritic agent. In some embodiments, the subject has a total daily urine volume of no more than about 9500 ml upon administration of the anti-pruritic agent. In one embodiment, the subject has a total daily urine volume of no more than about 9000 ml upon administration of the anti-pruritic agent. In another embodiment, the subject has a total daily urine volume of no more than about 8650 ml upon administration of the anti-pruritic agent. In yet another embodiment, the subject has a total daily urine volume of about 500 ml to about 9500 ml upon administration of the anti-pruritic agent. In yet another embodiment, the subject has a total daily urine volume of about 650 ml to about 9500 ml upon administration of the anti-pruritic agent. In yet another embodiment, the subject has a total daily urine volume of about 700 ml to about 9000 ml upon administration of the anti-pruritic agent. In certain embodiments, the subject has a total daily urine volume of about 750 ml to about 8650 ml upon administration of the anti-pruritic agent.

In one embodiment, the method of treating pruritus comprising administering an effective amount of an anti-pruritic agent to a subject does not significantly alter the urine specific gravity in the subject upon administration. In some embodiments, the urine specific gravity in the subject ranges from about 0.900 to about 1.133 upon administration of the anti-pruritic agent. In some other embodiments, the urine specific gravity in the subject ranges from about 0.951 to about 1.0815 upon administration of the anti-pruritic agent. In one embodiment, the urine specific gravity in the subject ranges from about 1.001 to about 1.030 upon administration of the anti-pruritic agent.

In one embodiment, the method of treating pruritus comprising administering an effective amount of an anti-pruritic agent to a subject does not significantly alter the urobilinogen content of urine in the subject upon administration of the anti-pruritic agent. In some embodiments, the urobilinogen content of urine in the subject ranges from about 0 to about 21 μmol/L upon administration of the anti-pruritic agent. In some other embodiments, the urobilinogen content of urine in the subject ranges from about 0 to about 20 μmol/L upon administration of the anti-pruritic agent. In certain other embodiments, the urobilinogen content of urine in the subject ranges from about 0 to about 19 μmol/L upon administration of the anti-pruritic agent.

In one embodiment, the method of treating pruritus comprising administering an effective amount of an anti-pruritic agent to a subject does not significantly alter the pH of urine in the subject upon administration. In one embodiment, the pH of urine in the subject ranges from about 4.5 to about 8.8 upon administration of the anti-pruritic agent. In another embodiment, the pH of urine in the subject ranges from about 4.7 to about 8.4 upon administration of the anti-pruritic agent. In yet another embodiment, the pH of urine in the subject ranges from about 5.0 to about 8.0 upon administration of the anti-pruritic agent.

In one embodiment, the method of treating pruritus comprising administering an effective amount of an anti-pruritic agent to a subject does not significantly alter the serum sodium content of the subject upon administration of the anti-pruritic agent. In some embodiments, the method of the invention does not induce hypernatremia in the subject upon administration of the anti-pruritic agent. In one embodiment, the serum sodium content of the subject ranges from about 122 to about 161 mmol/L upon administration of the anti-pruritic agent. In other embodiments, the serum sodium content of the subject ranges from about 129 to about 154 mmol/L upon administration of the anti-pruritic agent. In some other embodiments, the serum sodium content of the subject ranges from about 136 to about 146 mmol/L upon administration of the anti-pruritic agent. In yet some embodiments, the serum sodium content of the subject is no more than about 161 mmol/L, no more than about 153 mmol/L, or no more than about 146 mmol/L after administration of the anti-pruritic agent.

In one embodiment, the method of treating pruritus comprising administering an effective amount of an anti-pruritic agent to a subject does not significantly alter the heart rate of the subject upon administration of the anti-pruritic agent. In one embodiment, the hear rate of the subject ranges from about 45 to about 113 beats/min upon administration of the anti-pruritic agent, provided that the subject does not need hemodialysis. In another embodiment, the hear rate of the subject ranges from about 47 to about 107 beats/min upon administration of the anti-pruritic agent, provided that the subject does not need hemodialysis. In yet another embodiment, the hear rate of the subject ranges from about 50 to about 102 beats/min upon administration of the anti-pruritic agent, provided that the subject does not need hemodialysis. In some embodiments, the heart rate of the subject is no more than about 112 beats/min, no more than about 108 beats/min, or no more than about 102 beats/min upon administration of the anti-pruritic agent, provided that the subject does not need hemodialysis.

In some embodiments, the hear rate of the subject ranges from about 41 to about 140 beats/min upon administration of the anti-pruritic agent, provided that the subject needs hemodialysis. In some other embodiments, the hear rate of the subject ranges from about 43 to about 133 beats/min upon administration of the anti-pruritic agent, provided that the subject needs hemodialysis. In still some other embodiments, the hear rate of the subject ranges from about 46 to about 126 beats/min upon administration of the anti-pruritic agent, provided that the subject needs hemodialysis. In yet some embodiments, the heart rate of the subject is no more than about 139 beats/min, no more than about 132 beats/min, or no more than about 126 beats/min upon administration of the anti-pruritic agent, provided that the subject needs hemodialysis.

In one embodiment, the method of treating pruritus comprising administering an effective amount of an anti-pruritic agent to a subject does not significantly alter blood pressure of the subject upon administration of the anti-pruritic agent. In some embodiments, the methods of the invention do not induce hypotension in the subject upon administration of the anti-pruritic agent.

In one embodiment, the systolic blood pressure ranges from about 70 to about 163 mmHg upon administration of the anti-pruritic agent in a subject that does not need hemodialysis. In another embodiment, the systolic blood pressure ranges from about 74 to about 156 mmHg upon administration of the anti-pruritic agent in a subject that does not need hemodialysis. In yet another embodiment, the systolic blood pressure ranges from about 78 to about 148 mmHg upon administration of the anti-pruritic agent in a subject that does not need hemodialysis.

In one embodiment, the systolic blood pressure ranges from about 63 to about 220 mmHg upon administration of the anti-pruritic agent in a subject that needs hemodialysis. In another embodiment, the systolic blood pressure ranges from about 66 to about 209 mmHg upon administration of the anti-pruritic agent in a subject that needs hemodialysis. In yet another embodiment, the systolic blood pressure ranges from about 70 to about 199 mmHg upon administration of the anti-pruritic agent in a subject that needs hemodialysis.

In some embodiments, the diastolic blood pressure ranges from about 37 to about 103 mmHg upon administration of the anti-pruritic agent in a subject that does not need hemodialysis. In some other embodiments, the diastolic blood pressure ranges from about 40 to about 98 mmHg upon administration of the anti-pruritic agent in a subject that does not need hemodialysis. In still some other embodiments, the diastolic blood pressure ranges from about 42 to about 93 mmHg upon administration of the anti-pruritic agent in a subject that does not need hemodialysis.

In some embodiments, the diastolic blood pressure ranges from about 30 to about 120 mmHg upon administration of the anti-pruritic agent in a subject that needs hemodialysis. In some other embodiments, the diastolic blood pressure ranges from about 32 to about 115 mmHg upon administration of the anti-pruritic agent in a subject that needs hemodialysis. In still some other embodiments, the diastolic blood pressure ranges from about 34 to about 109 mmHg upon administration of the anti-pruritic agent in a subject that needs hemodialysis.

In one embodiment, the subject is suffering from a pruritic condition, and said pruritic condition comprises atopic dermatitis, nervous dermatitis, contact dermatitis, seborrheic dermatitis, autosensitization dermatitis, caterpillar dermatitis, asteatosis, senile pruritus cutaneous, insect sting, photosensitive dermatosis, urticaria, prurigo, herpes, impetigo, eczema, tinea, lichen, psoriasis, scabies and acne vulgaris, or visceral diseases complicated with pruritus, bullous pemphigoid, Netherton syndrome, and itching from poison or any other stimulus, e.g., poison oak, poison ivy, etc.

In another embodiment, the subject is suffering from a skin change comprising pruritus secondary to inflamed skin, pruritus arising from conditions of non-diseased skin, pruritus associated with chronic secondary scratch, or skin lesions resulting from an underlying medical condition.

In yet another embodiment, the subject has uremic pruritus or prurigo nodularis.

In some embodiments, the anti-pruritus agent is administered at an initial oral dose of from about 15 mg to about 30 mg once or twice a day and then titrated to an effective dose.

In some other embodiments, the anti-pruritus agent is administered at an initial dose of from about 15 mg to about 30 mg twice a day or once a day for about 2-3 days and then titrated to an effective dose at about 15 mg to about 30 mg increment.

In yet some other embodiments, the maximum dose of the anti-pruritus agent is about 480 mg when said agent is administered to a subject twice a day or about 240 mg when said agent is administered to a subject once a day.

In some embodiments, the anti-pruritus agent is administered with an AM dosage and a PM dosage and wherein the PM dosage is higher than the AM dosage, or vice versa.

In some other embodiments, the anti-pruritus agent is administered at a dose of about 60 mg or about 120 mg twice a day to a subject with uremic pruritus or renal impairment or about 90 mg or about 180 mg twice a day to a subject without a renal impairment condition.

In some embodiments, the anti-pruritus agent is in an extended release oral dosage form and the administration provides in the subject a mean Cₘₐₓ of from about 1 ng/mL to about 90 ng/mL, from about 5 ng/mL to about 85 ng/mL, from about 5 ng/ml to about 45 ng/ml, from about 25 ng/mL to about 72 ng/mL, or from about 13 ng/mL to about 28 ng/mL.

In some other embodiments, the anti-pruritus agent is in an extended release oral dosage form and the administration provides in the subject an AUC_{(0-∞)} of from about 40 ng·hr/mL to about 3000 ng·hr/mL, 40 ng·hr/mL to about 800 ng·hr/mL or 30 ng·hr/mL to about 360 ng·hr/mL.

In some embodiments, the anti-pruritus agent is in an extended release oral dosage form.

In some other embodiments, the administration provides in the subject a pK release profile with the characteristics of a) a mean Cₘₐₓ from about 1.5 ng/mL to about 195 ng/mL, and b) AUC_{(0-∞)} from about 20 ng·hr/mL to about 4100 ng·hr/mL.

In yet some other embodiments, the administration provides in the subject a pK release profile with the characteristics of a) a mean Cₘₐₓ from about 1.5 ng/mL to about 60 ng/mL, and b) AUC_{(0-∞)} from about 20 ng·hr/mL to about 700 ng·hr/mL.

The present methods, and advantages thereof, are further illustrated by the following non-limiting detailed description and Examples.

### BRIEF DESCRIPTION OF THE DRAWINGS

FIG. 1 is a graphical representation of urine volume in individual healthy subjects over 24 hours as a function of dose and dosing day.

FIG. 2 is a graphical representation of mean heart rate (SD, N=9) in healthy subjects over 24 hours before treatment and following nalbuphine HCl 180 mg BID dose on Day 12.

FIG. 3 is a graphical representation of mean heart rate (SD, N=14) in hemodialysis patients over 24 hours before treatment and following nalbuphine HCl 180 mg BID dose on a non-dialysis day (Day 11) or dialysis day (Day 12). The “24-hr Post dose” time point corresponds to the following day's pre-dose value.

### DETAILED DESCRIPTION

The term “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The terms “administer,” “administering” or “administration” as used herein refer to either directly administering a compound or pharmaceutically acceptable salt of the compound or a composition to a subject.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound, or a salt, solvate or ester thereof, that, when administered to a subject, is capable of performing the intended result. For example, an effective amount of an anti-pruritic agent is that amount which is required to reduce at least one symptom of pruritus in a subject, e.g. the amount required to reduce the itching sensation in a subject. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The term “therapeutic effect” as used herein refers to a desired or beneficial effect provided by the method and/or the composition. For example, the method for treating pruritus provides a therapeutic effect when the method reduces at least one symptom of pruritus, e.g., itching sensation, in a subject.

The term “aquaretic effect” as used herein refers to the increased urine output or production (diuresis) and/or changes in urine specific gravity, serum sodium levels, blood pressure or heart rate associated with increased urine output. According to one aspect, the phrase “without producing substantial aquaretic effect” means the methods of the invention do not increase urine output or alter urine specific gravity, serum sodium levels, blood pressure or heart rate in a subject upon administration of an anti-pruritic agent. That is, according to the invention, urine output, urine specific gravity, serum sodium levels, blood pressure and/or heart rate of a subject prior to, during, and after treatment do not change significantly or remain substantially similar. For instance, according to one aspect, the phrase “without producing substantial aquaretic effect” means a urine output in a subject prior to and upon administration of an anti-pruritic agent ranges from about 400 ml to about 9000 ml, about 500 ml to about 9500 ml, about 650 ml to about 9500 ml, about 700 ml to about 9000 ml, or about 750 ml to about 8650 ml, including values and ranges therebetween. According to additional aspects, urine output in a subject prior to and upon administration of an anti-pruritic agent ranges from about 500 ml to about 8500 ml, about 500 ml to about 8000 ml, about 500 ml to about 7500 ml, about 500 ml to about 7000 ml, about 500 ml to about 6500 ml, about 500 ml to about 6000 ml, about 500 ml to about 5500 ml, about 500 ml to about 5000 ml, about 500 ml to about 4500 ml, about 500 ml to about 4000 ml, about 500 ml to about 3500 ml, about 500 ml to about 3000 ml, about 500 ml to about 2500 ml, or about 500 ml to about 2000 ml, including values and ranges therebetween. According to another aspect, urine specific gravity in a subject prior to and upon administration of an anti-pruritic agent ranges from about 0.900 to about 1.133, about 0.951 to about 1.0815, or about 1.000 to 1.030.

According to yet another aspect, the phrase “without producing substantial aquaretic effect” means the methods of the invention do not significantly alter the urobilinogen content of urine. For instance, the urobilinogen content of urine in a subject prior to and upon administration of an anti-pruritic agent ranges from about 0 to about 21 μmol/L, 0 to about 20 μmol/L, or 0 to about 19 μmol/L, including values and ranges therebetween. According to yet another aspect, pH of the urine in a subject prior to and upon administration of an anti-pruritic agent remains substantially unaltered and ranges from about 4.5 to about 8.8, about 4.7 to about 8.4, or about 5.0 to about 8.0, including values and ranges therebetween. According to yet another aspect, serum sodium levels in a subject prior to and upon administration of an anti-pruritic agent range from about 122 to about 161 mmol/L, about 129 to about 154 mmol/L, or about 136 mmol/L to about 146 mmol/L, including values and ranges therebetween. According to yet another aspect, the phrase “without producing substantial aquaretic effect” means the methods of the invention do not induce hypernatremia in the subject upon administration of an anti-pruritic agent. In one embodiment, the serum sodium content of the subject prior to and upon administration of an anti-pruritic agent is no more than about 161 mmol/L, about 153 mmol/L, or about 146 mmol/L.

According to yet another aspect, the phrase “without producing substantial aquaretic effect” means the diastolic blood pressure in a subject that does not need hemodialysis ranges from about 37 to about 103 mmHg, about 40 to about 98 mmHg, or about 42 to about 93 mmHg prior to and upon administration of an anti-pruritic agent. According to yet another aspect, the diastolic blood pressure in a subject that needs hemodialysis ranges from about 30 to about 120 mmHg, about 32 to about 115 mmHg, or about 34 to about 109 mmHg prior to and upon administration of an anti-pruritic agent. According to yet another aspect, the systolic blood pressure in a subject that does not need hemodialysis ranges from about 70 to about 163 mmHg, about 74 to about 156 mmHg, or about 78 to about 148 mmHg prior to and upon administration of an anti-pruritic agent. According to yet another aspect, the systolic blood pressure in a subject that needs hemodialysis ranges from about 63 to about 220 mmHg, about 66 to about 209 mmHg, or about 70 to about 199 mmHg prior to and upon administration of an anti-pruritic agent. According to yet another aspect, the heart rate in a subject that does not need hemodialysis ranges from about 45 to about 113 beats/min, about 47 to about 107 beats/min, or about 50 to about 102 beats/min prior to and upon administration of an anti-pruritic agent. According to yet another aspect, the heart rate in a subject that needs hemodialysis ranges from about 41 to about 140 beats/min, about 43 to about 133 beats/min, or about 46 to about 126 beats/min prior to and upon administration of an anti-pruritic agent.

The term “urine output” or “daily urine output” refers to the amount of urine produced by a subject in a day or in 24 hours.

The term “hypernatremia” refers to increased serum sodium levels caused due to a decrease in total body water. According to one aspect, hypernatremia refers to a rise in serum sodium concentration to a value above 146 mmol/L.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used in this disclosure, the term “subject” includes, without limitation, a human or an animal. Exemplary animals include, but are not limited to, mammals such as mouse, rat, guinea pig, dog, cat, horse, cow, pig, monkey, chimpanzee, baboon, or rhesus monkey.

The term “treating” as used herein with regard to a subject, refers to improving at least one symptom of the subject's disorder. Treating can be curing, improving, or at least partially ameliorating a disorder.

### Methods for Treating Pruritus

In one aspect, the present invention provides a method of treating pruritus comprising administering an effective amount of an anti-pruritus agent to a subject in need of such treatment, wherein the anti-pruritus agent is nalbuphine or a pharmaceutically acceptable salt, solvate or ester thereof and the method provides a therapeutic effect without producing a substantial aquaretic effect.

Certain drugs that interact with opioid receptors are known to have aquaretic effects, i.e. they increase the urine output in a subject upon administration. Aquaretic effects are not desirable in normal healthy subjects as aquaresis can lead to dehydration or reduced total body water levels. Aquaretic effects are also not desirable in subjects with compromised renal function (e.g. subjects with chronic kidney disease, uremic pruritus, including dialysis patients) as aquaresis would put burden on fewer nephrons available in these subjects. In various embodiments, the present invention provides methods for treating pruritus comprising administering an effective amount of nalbuphine or a pharmaceutically acceptable salt, solvate or ester thereof to a subject in need of such treatment, wherein the methods provide a therapeutic effect without producing a substantial aquaretic effect. In one embodiment, the subject in need of a treatment of pruritus is a subject with compromised renal function. In another embodiment, the subject in need of a treatment of pruritus is a subject with uremic pruritus. In yet another embodiment, the subject in need of a treatment of pruritus is a subject with chronic kidney disease. In yet another embodiment, the subject in need of a treatment of pruritus is a subject who needs hemodialysis.

According to one aspect of the invention, treating pruritus in a subject by administering nalbuphine or a pharmaceutically acceptable salt, solvate or ester thereof does not alter the total daily (24-hour) urine volume of the subject significantly. That is, the total daily urine volume (also known as urine output) in a subject prior to and upon treatment with the methods of the invention remains substantially the same. The total daily urine volume in a subject prior to and upon treatment with the methods of the invention may range from about 750 ml to about 8650 ml, including values and ranges therebetween. In another embodiment, the total daily urine volume in a subject prior to and upon treatment with the methods of the invention may range from about 700 ml to about 9000 ml, including values and ranges therebetween. In still another embodiment, the total daily urine volume in a subject prior to and upon treatment with the methods of the invention may range from about 650 ml to about 9500 ml, including values and ranges therebetween. In certain other embodiments, the total daily urine volume in a subject prior to and upon treatment with the methods of the invention may range from about 500 ml to about 9500 ml, about 500 ml to about 9000 ml, about 600 ml to about 9300 ml, about 700 ml to about 9000 ml, about 750 ml to about 9000 ml, about 800 ml to about 8800 ml, about 850 ml to about 8650 ml, about 900 ml to about 8500 ml, about 1000 ml to about 8500 ml, about 1250 ml to about 8250 ml, about 1400 ml to about 8100 ml, about 1500 ml to about 7900 ml, about 500 ml to about 7500 ml, about 500 ml to about 7250 ml, about 700 ml to about 7000 ml, about 500 ml to about 6500 ml, about 500 ml to about 6250 ml, about 500 ml to about 6000 ml, about 500 ml to about 5750 ml, about 500 ml to about 5500 ml, about 500 ml to about 5250 ml, about 500 ml to about 5000 ml, about 500 ml to about 4750 ml, about 500 ml to about 4500 ml, about 500 ml to about 4250 ml, about 500 ml to about 4000 ml, about 500 ml to about 3750 ml, about 500 ml to about 3500 ml, about 500 ml to about 3250 ml, about 500 ml to about 3000 ml, about 500 ml to about 2750 ml, about 500 ml to about 2500 ml, about 500 ml to about 2250 ml, or about 500 ml to about 2000 ml, including values and ranges therebetween. In yet other embodiments, the total daily urine volume in a subject prior to and upon treatment with the methods of the invention is at least 500 ml. In yet other embodiments, the total daily urine volume in a subject prior to and upon treatment with the methods of the invention is not more than about 1000 ml, about 1500 ml, about 2000 ml, about 2500 ml, about 3000 ml, about 3500 ml, about 4000 ml, about 4500 ml, about 5000 ml, about 5500 ml, about 6000 ml, about 6500 ml, about 7000 ml, about 7500 ml, about 8000 ml, about 8500 ml, about 9000 ml, or about 9500 ml.

According to another aspect, treating pruritus in a subject by administering nalbuphine or a pharmaceutically acceptable salt, solvate or ester thereof does not significantly alter the urine specific gravity in a subject. That is, the urine specific gravity of the subject prior to and upon administration of nalbuphine or a pharmaceutically acceptable salt, solvate or ester thereof according to the methods of the invention remains substantially the same. In one embodiment, the urine specific gravity in the subject prior to and upon treatment with the methods of the invention ranges from about 1.001 to about 1.030, including values and ranges therebetween. In another embodiment, the urine specific gravity in the subject prior to and upon treatment with the methods of the invention ranges from about 0.951 to about 1.0815, including values and ranges therebetween. In yet another embodiment, the urine specific gravity in the subject prior to and upon treatment with the methods of the invention ranges from about 0.900 to about 1.133, including values and ranges therebetween.

According to yet another aspect, treating pruritus in a subject by administering nalbuphine or a pharmaceutically acceptable salt, solvate or ester thereof does not significantly alter the urobilinogen content of urine in a subject. That is, the urobilinogen content of urine in the subject prior to and upon administration of nalbuphine or a pharmaceutically acceptable salt, solvate or ester thereof according to the methods of the invention remains substantially the same. In one embodiment, the urobilinogen content of urine in the subject prior to and upon treatment with the methods of the invention ranges from about 0 to about 19 μmol/L, including values and ranges therebetween. In another embodiment, the urobilinogen content of urine in the subject prior to and upon treatment with the methods of the invention ranges from about 0 to about 20 μmol/L, including values and ranges therebetween. In yet another embodiment, the urobilinogen content of urine in the subject prior to and upon treatment with the methods of the invention ranges from about 0 to about 21 μmol/L, including values and ranges therebetween.

According to yet another aspect, treating pruritus in a subject by administering nalbuphine or a pharmaceutically acceptable salt, solvate or ester thereof does not significantly alter the pH of urine in a subject. That is, the pH of urine in the subject prior to and upon administration of nalbuphine or a pharmaceutically acceptable salt, solvate or ester thereof according to the methods of the invention remains substantially the same. In one embodiment, the pH of urine in the subject prior to and upon treatment with the methods of the invention ranges from about 5.0 to about 8.0, including values and ranges therebetween. In another embodiment, the pH of urine in the subject prior to and upon treatment with the methods of the invention ranges from about 4.7 to about 8.4, including values and ranges therebetween. In yet another embodiment, the pH of urine in the subject prior to and upon treatment with the methods of the invention ranges from about 4.5 to about 8.8, including values and ranges therebetween.

According to yet another aspect, treating pruritus in a subject by administering nalbuphine or a pharmaceutically acceptable salt, solvate or ester thereof does not significantly alter the serum sodium content of a subject. That is, the serum sodium content of the subject prior to and upon administration of nalbuphine or a pharmaceutically acceptable salt, solvate or ester thereof according to the methods of the invention remains substantially the same. In one embodiment, the serum sodium content of the subject prior to and upon treatment with the methods of the invention ranges from about 136 to about 146 mmol/L, including values and ranges therebetween. In another embodiment, the serum sodium content of the subject prior to and upon treatment with the methods of the invention ranges from about 129 to about 154 mmol/L, including values and ranges therebetween. In yet another embodiment, the serum sodium content of the subject prior to and upon treatment with the methods of the invention ranges from about 122 to about 161 mmol/L, including values and ranges therebetween.

In some embodiments, the methods of the invention do not induce hypernatremia in the subject upon administration of an anti-pruritic agent. In one embodiment, the serum sodium content of the subject prior to and upon treatment with the anti-pruritic agent is no more than 146 mmol/L. In another embodiment, the serum sodium content of the subject prior to and upon treatment with the anti-pruritic agent is no more than 153 mmol/L. In yet another embodiment, the serum sodium content of the subject prior to and upon treatment with the anti-pruritic agent is no more than 161 mmol/L.

According to yet another aspect, treating pruritus in a subject by administering nalbuphine or a pharmaceutically acceptable salt, solvate or ester thereof does not significantly alter the heart rate of the subject. That is, the heart rate of the subject prior to and upon administration of nalbuphine or a pharmaceutically acceptable salt, solvate or ester thereof according to the methods of the invention remains substantially the same. In one embodiment, the heart rate of the subject prior to and upon treatment with the methods of the invention ranges from about 50 to about 102 beats per minute, including values and ranges therebetween, provided that the subject does not need hemodialysis. In another embodiment, the heart rate of the subject prior to and upon treatment with the methods of the invention ranges from about 47 to about 107 beats per minute, including values and ranges therebetween, provided that the subject does not need hemodialysis. In yet another embodiment, the heart rate of the subject prior to and upon treatment with the methods of the invention ranges from about 45 to about 113 beats per minute, including values and ranges therebetween, provided that the subject does not need hemodialysis. In some embodiments, the average heart rate of the subject prior to and upon treatment with the methods of the invention is no more than about 73 beats per minute, about 77 beats per minute, about 81 beats per minute, about 85 beats per minute, about 88 beats per minutes, about 92 beats per minutes, about 96 beats per minutes, about 100 beats per minute, about 102 beats per minute, about 105 beats per minutes, about 108 beats per minutes, about 112 beats per minutes, or about 115 beats per minutes, provided that the subject does not need hemodialysis.

In another embodiment, the heart rate of the subject prior to and upon treatment with the methods of the invention ranges from about 46 to about 126 beats per minute, including values and ranges therebetween, provided that the subject needs hemodialysis. In yet another embodiment, the heart rate of the subject prior to and upon treatment with the methods of the invention ranges from about 43 to about 133 beats per minute, including values and ranges therebetween, provided that the subject needs hemodialysis. In yet another embodiment, the heart rate of the subject prior to and upon treatment with the methods of the invention ranges from about 41 to about 140 beats per minute, including values and ranges therebetween, provided that the subject needs hemodialysis. In some embodiments, the average heart rate of the subject prior to and upon treatment with the methods of the invention is no more than about 104 beats per minute, about 109 beats per minute, about 115 beats per minute, about 120 beats per minute, about 125 beats per minutes, about 130 beats per minutes, about 132 beats per minutes, about 135 beats per minutes, or about 139 beats per minute, provided that the subject needs hemodialysis.

According to yet another aspect, treating pruritus in a subject by administering nalbuphine or a pharmaceutically acceptable salt, solvate or ester thereof does not significantly alter the blood pressure of the subject. That is, the blood pressure of the subject prior to and upon administration of nalbuphine or a pharmaceutically acceptable salt, solvate or ester thereof according to the methods of the invention remains substantially the same. In one embodiment, the methods of the invention do not induce hypotension in the subject upon administration of an anti-pruritic agent. In another embodiment, the methods of the invention do not substantially lower the blood pressure of the subject upon administration of an anti-pruritic agent.

In one embodiment, the diastolic blood pressure of the subject prior to and upon treatment with the methods of the invention ranges from about 42 to about 93 mmHg, including values and ranges therebetween, provided that the subject does not need hemodialysis. In another embodiment, the diastolic blood pressure of the subject prior to and upon treatment with the methods of the invention ranges from about 40 to about 98 mmHg, including values and ranges therebetween, provided that the subject does not need hemodialysis. In yet another embodiment, the diastolic blood pressure of the subject prior to and upon treatment with the methods of the invention ranges from about 37 to about 103 mmHg, including values and ranges therebetween, provided that the subject does not need hemodialysis.

In one embodiment, the diastolic blood pressure of the subject prior to and upon treatment with the methods of the invention ranges from about 34 to about 109 mmHg, including values and ranges therebetween, provided that the subject needs hemodialysis. In another embodiment, the diastolic blood pressure of the subject prior to and upon treatment with the methods of the invention ranges from about 32 to about 115 mmHg, including values and ranges therebetween, provided that the subject needs hemodialysis. In yet another embodiment, the diastolic blood pressure of the subject prior to and upon treatment with the methods of the invention ranges from about 30 to about 120 mmHg, including values and ranges therebetween, provided that the subject needs hemodialysis.

In one embodiment, the systolic blood pressure of the subject prior to and upon treatment with the methods of the invention ranges from about 78 to about 148 mmHg, including values and ranges therebetween, provided that the subject does not need hemodialysis. In another embodiment, the systolic blood pressure of the subject prior to and upon treatment with the methods of the invention ranges from about 74 to about 156 mmHg, including values and ranges therebetween, provided that the subject does not need hemodialysis. In yet another embodiment, the systolic blood pressure of the subject prior to and upon treatment with the methods of the invention ranges from about 70 to about 163 mmHg, including values and ranges therebetween, provided that the subject does not need hemodialysis.

In one embodiment, the mean systolic blood pressure of the subject prior to and upon treatment with the methods of the invention ranges from about 108 to about 128 mmHg, including values and ranges therebetween, provided that the subject does not need hemodialysis. In another embodiment, the mean systolic blood pressure of the subject prior to and upon treatment with the methods of the invention ranges from about 102 to about 135 mmHg, including values and ranges therebetween, provided that the subject does not need hemodialysis. In yet another embodiment, the mean systolic blood pressure of the subject prior to and upon treatment with the methods of the invention ranges from about 97 to about 141 mmHg, including values and ranges therebetween, provided that the subject does not need hemodialysis.

In one embodiment, the systolic blood pressure of the subject prior to and upon treatment with the methods of the invention ranges from about 70 to about 199 mmHg, including values and ranges therebetween, provided that the subject needs hemodialysis. In another embodiment, the systolic blood pressure of the subject prior to and upon treatment with the methods of the invention ranges from about 66 to about 209 mmHg, including values and ranges therebetween, provided that the subject needs hemodialysis. In yet another embodiment, the systolic blood pressure of the subject prior to and upon treatment with the methods of the invention ranges from about 63 to about 220 mmHg, including values and ranges therebetween, provided that the subject needs hemodialysis.

In one embodiment, the mean systolic blood pressure of the subject prior to and upon treatment with the methods of the invention ranges from about 106 to about 158 mmHg, including values and ranges therebetween, provided that the subject needs hemodialysis. In another embodiment, the mean systolic blood pressure of the subject prior to and upon treatment with the methods of the invention ranges from about 100 to about 166 mmHg, including values and ranges therebetween, provided that the subject needs hemodialysis. In yet another embodiment, the mean systolic blood pressure of the subject prior to and upon treatment with the methods of the invention ranges from about 95 to about 174 mmHg, including values and ranges therebetween, provided that the subject needs hemodialysis.

In one embodiment, the anti-pruritic agent administered to a subject is nalbuphine or a pharmaceutically acceptable salt, solvate or ester thereof “Nalbuphine” includes nalbuphine free base, metabolites thereof, derivatives thereof, solvates thereof (e.g., hydrates, alcoholates, etc.) and/or pharmaceutically acceptable salts or esters thereof. Metabolites of nalbuphine include, for example the glucuronide conjugate metabolite and metabolites resulting from methylation, oxidation/dehydrogenation, hydroxylation, double hydroxylation, triple hydroxylation, oxidative methylation, glucoside conjugation, glucuronide conjugation, and hydroxyl-glucuronide conjugation of nalbuphine. Exemplary metabolites can include nornalbuphine, 6-ketonalbuphine, nalbuphine 3-glucuronide. In one embodiment, metabolites include triple hydroxylated nalbuphine, mono-hydroxylated nalbuphine, and mono-glucuronidated nalbuphine. Isomers include the C-6β-epimer of nalbuphine (Mallinckrodt, Nalbuphine hydrochloride Technical Package August 2003). Derivatives of nalbuphine can include pharmaceutically acceptable ester prodrugs thereof (including alkoxy esters such as methoxy and ethoxy esters) which can be hydrolyzed in vivo to provide nalbuphine, as well as ether or other compounds prepared by, e.g. reacting the hydroxyl groups of nalbuphine with suitable protecting agents.

In one embodiment, nalbuphine suitable for use in the present methods is in the form of any pharmaceutically acceptable salt, solvate or ester known in the art. Exemplary pharmaceutically acceptable salts include without limitation hydrochloric, sulfuric, nitric, phosphoric, hydrobromic, maleic, malic, ascorbic, citric, tartaric, pamoic, lauric, stearic, palmitic, oleic, myristic, lauryl sulfuric, napthalinesulfonic, linoleic, linolenic acid, and the like. In one embodiment the anti-pruritus agent is the hydrochloride salt of nalbuphine.

The present invention also includes pharmaceutically acceptable esters of the anti-pruritic agent. The term “ester” denotes a derivative of the anti-pruritic agent containing an ester functional group (as described herein), which is capable of releasing the anti-pruritic agent when the ester form is administered to a subject. Release of the active ingredient occurs in vivo. Pharmaceutically acceptable esters can be prepared by techniques known to one skilled in the art. These techniques generally modify appropriate functional groups in a given compound. These modified functional groups however regenerate original functional groups by metabolism of the compound in vivo. Esters include compounds wherein a hydroxy, amino, carboxylic, or a similar group is modified.

Suitable pharmaceutically acceptable esters for a hydroxyl group include inorganic esters such as phosphate esters and α-acyloxyalkyl ethers and related compounds which, as a result of in vivo hydrolysis of the ester, provide the parent hydroxy group. In vivo hydrolyzable ester forming groups for hydroxy include alkanoyl (e.g., C₁₋₁₀ linear, branched or cyclic alkyl), benzoyl, phenylacetyl and substituted benzoyl and phenylacetyl, alkoxycarbonyl (to give alkyl carbonate esters), dialkylcarbamoyl and N—(N,N-dialkylaminoethyl)-N-alkyl carbamoyl (to give carbamates), N,N-dialkylaminoacetyl and carboxyacetyl.

Nalbuphine as employed in the present methods can form a part of a pharmaceutical composition by combining nalbuphine, or a pharmaceutically acceptable salt, solvate or ester thereof, with a pharmaceutically acceptable carrier. Additionally, the compositions can include an additive selected from the group consisting of adjuvants, excipients, diluents, release-modifying agents and stabilizers. The composition can be an immediate release formulation, a delayed release formulation, a sustained release formulation or an extended release formulation.

### Nalbuphine

Nalbuphine HCl (17-(cyclobutylmethyl)-4,5α-epoxymorphinian-3,6α,14-triol, hydrochloride) is a synthetic opioid. Structurally, nalbuphine is a derivative of 14 hydroxymorphine.

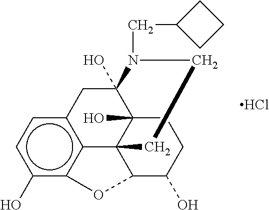

Nalbuphine HCl is currently available only as a generic medication in an injectable form. An injectable form of nalbuphine has been available as an approved drug formulation since 1978. Nubain® was the innovator brand injectable form of nalbuphine on which the presently sold generic bioequivalent injectable formulations are based. The injectable formulation is currently approved for use in the relief of moderate to severe pain, a supplement to balanced anesthesia, for pre-operative and post-operative analgesia and obstetrical analgesia during labor and delivery.

### Opioid Receptors

There are three classical types of opioid receptors that have been investigated as the mediators of opiate effects. These opioid receptors are classified as mu (“μ”), kappa (“κ”) and delta (“δ”). Nalbuphine is a derivative of 14-hydroxymorphine and is structurally related to the opioid μ-receptor agonist oxymorphone and the opioid μ-receptor antagonist naloxone. Gutstein et al. (Chapter 23: Opioid Analgesics, Goodman & Gilman's The Pharmacologic Basis of Therapeutics, 10th Ed., McGraw Hill 2001, pp 569-619) report that nalbuphine exerts its clinical pharmacologic action by competitively antagonizing the opioid μ-receptor and simultaneously acting as an agonist at the opioid κ-receptor, and thus is a member of the “opioid agonist-antagonist” class of drugs that mechanistically work through this dual pharmacologic process. Subsequent in vitro work by Gharagozlou et al. (Neurosci. 2002 3:19) showed that nalbuphine is in addition a δ opioid receptor antagonist. Gutstein et al. (supra) state that the stimulus for the development opioid agonist-antagonist drugs was to identify a drug with analgesic properties with less respiratory depression and addiction potential.

### Pruritic Conditions

According to the present invention, pruritus includes any itchy or pruritic condition, e.g., a sensation that causes the desire or reflex to scratch. In some embodiments, methods of the present invention are used for the treatment of a subject suffering from a pruritic condition selected from the group consisting of atopic dermatitis, nervous dermatitis, contact dermatitis, seborrheic dermatitis, autosensitization dermatitis, caterpillar dermatitis, asteatosis, senile pruritus cutaneous, insect sting, photosensitive dermatosis, urticarial, prurigo, herpes, impetigo, eczema, tinea, lichen, psoriasis, scabies and acne vulgaris, bullous pemphigoid, Netherton syndrome, itching from poison or any other stimulus, e.g., poison oak, poison ivy, etc., and visceral diseases complicated with pruritus such as malignant tumors, diabetes mellitus, hepatic diseases, renal failure, hemodialysis, peritoneal dialysis, and/or pregnancy.

In some embodiments, methods of the present invention are used for the treatment of a subject suffering from a pruritic condition associated with a skin change. For example, such pruritic condition can be selected from the group consisting of pruritus secondary to inflamed skin (e.g., atopic dermatitis, psoriasis, burns); pruritus arising from conditions of non-diseased skin (e.g., uremic pruritus, cholestatic pruritus, cancers, hydroxyethyl starch induced pruritus), and pruritus associated with chronic secondary scratch or other types of skin lesions that may or may not be the result of an underlying medical condition (e.g., prurigo nodularis) and the underlying disease is categorized based on histological, radiological or other investigations as being of an origin selected from the group consisting of dermatologic origin, systemic disease origin, neurologic origin, psychogenic origin, mixed origin, or other origin.

In some embodiments, methods of the present invention are used for the treatment of a subject suffering from a pruritic condition associated with neurogenic inflammation of the skin, e.g., prurigo nodularis, atopic dermatitis, burn pruritus, burn, wound healing, etc. In some other embodiments, methods of the present invention are used for the treatment of a subject suffering from a pruritic condition associated with neurogenic inflammation with elevated substance P level. In still some other embodiments, methods of the present invention are used for the treatment of a subject suffering from a pruritic condition associated with elevated substance P level.

In some embodiments, methods of the present invention are used for the treatment of a subject suffering from a pruritic condition associated with one or more related or unrelated conditions. For example, the pruritic condition can be associated with a dermatologic condition including aquagenic pruritus, atopic dermatitis, idiopathic pruritus, Lichen simplex chronicus, prurigo nodularis, psoriasis, and scabies. In another example, the pruritic condition can be associated with a hematological or oncological condition including cancer related pruritus, chemotherapy induced pruritus, HIV protease inhibitor induced pruritus, Hodgkin's lymphoma associated pruritus, polycythemia vera, etc. In another example, the pruritic condition can be associated with a metabolic condition including cholestatic pruritus, uremic pruritus, etc. In still another example, the pruritic condition can be associated with a condition of pain or neurological condition including brachioradial pruritus, burn induced pruritus, neuropathic pruritus, morphine induced pruritus, multiple sclerosis associated pruritus, post herpetic pruritus, pruritus associated with psychiatric causes, etc.

In one embodiment, methods of the present invention are used for the treatment of uremic pruritus. In another embodiment, methods of the present invention are used for the treatment of prurigo nodularis. In certain embodiments, Nalbuphine HCl is used or indicated for the treatment of itch in adult patients with end-stage-renal disease on hemodialysis with moderate to severe uremic pruritus. In certain embodiments, Nalbuphine HCl is used or indicated for the treatment of itch in adult patients with moderate to severe prurigo nodularis.

In yet another embodiment, methods of the present invention are used to treat human beings. In still another embodiment, methods of the present invention are used to treat animals other than human beings.

### Formulations

The methods of the present invention can employ various formulations for administration to subjects, e.g., humans and animals in unit dosage forms, such as tablets, capsules, pills, powders, granules, sterile parenteral solutions or suspensions, and oral solutions or suspensions, and oil-water emulsions containing suitable quantities of an anti-pruritic agent, e.g., nalbuphine, or pharmaceutically acceptable salts or esters thereof.

Oral pharmaceutical dosage forms can be either solid or liquid. The solid dosage forms can be tablets, capsules, granules, and bulk powders. Types of oral tablets include compressed, chewable lozenges and tablets which can be enteric-coated, sugar-coated or film-coated. Capsules can be hard or soft gelatin capsules, while granules and powders can be provided in non-effervescent or effervescent form with the combination of other ingredients known to those skilled in the art. In other embodiments, the oral dosage form may be an osmotic-controlled release oral delivery system (OROS). In other embodiments, the oral dosage form may include matrix-embedded dosage forms or related devices. In some embodiments, the present oral dosage forms may include orally-disintegrating tablets.

Pharmaceutically acceptable carriers utilized in tablets include binders, lubricants, diluents, disintegrating agents, coloring agents, flavoring agents, and wetting agents.

Liquid oral dosage forms include aqueous solutions, emulsions, suspensions, solutions and/or suspensions reconstituted from non-effervescent granules and effervescent preparations reconstituted from effervescent granules.

Aqueous solutions include, for example, elixirs and syrups. Emulsions can be either oil-in water or water-in-oil. Elixirs are clear, sweetened, hydroalcoholic preparations. Pharmaceutically acceptable carriers used in elixirs include solvents. Syrups can be concentrated aqueous solutions of a sugar, for example, sucrose, and can contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions can use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substance used in effervescent granules, to be reconstituted into a liquid oral dosage form, can include organic acids and a source of carbon dioxide. Coloring and flavoring agents can be used in all of the above dosage forms.

Parenteral administration of the formulations of the present invention includes intravenous, subcutaneous and intramuscular administrations of immediate, sustained (e.g., depot), extended, and/or modified release formulations (e.g., as described herein). Preparations for parenteral administration include sterile solutions ready for injection, sterile dry soluble products ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous. Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

The concentration of the pharmaceutically active compound can be adjusted so that an injection provides an effective amount to produce the desired pharmacological effect. The exact dose depends on the age, weight and condition of the patient or animal, as is known in the art. The unit-dose parenteral preparations are packaged in an ampoule or a syringe with a needle. All preparations for parenteral administration must be sterile, as is known and practiced in the art. Illustratively, intravenous or intra-arterial infusion of a sterile aqueous solution containing an anti-pruritic agent is an effective mode of administration.

Pharmaceutical dosage forms for rectal administration can be rectal suppositories, capsules and tablets for systemic effect. Rectal suppositories as used herein mean solid bodies for insertion into the rectum which melt or soften at body temperature releasing the pharmacologically and/or therapeutically active ingredients contained in the composition of this invention. Pharmaceutically acceptable substances utilized in rectal suppositories are bases or vehicles and agents to raise the melting point. Examples of bases include cocoa butter (theobroma oil), glycerin-gelatin, carbowax, polyoxyethylene glycol and mixtures of mono-, di- and triglycerides of fatty acids. Combinations of the various bases can be used. Agents to raise the melting point of suppositories include spermaceti and wax. Rectal suppositories can be prepared either by the compressed method or by molding. The typical weight of a rectal suppository is about 2 to 3 gm. Tablets and capsules for rectal administration can be manufactured using the same pharmaceutically acceptable substance and by the same methods as for formulations for oral administration.

The compositions can be suspended in micronized or other suitable form or can be derivatized to produce a more soluble active product. The form of the resulting composition depends upon a number of factors, including the intended mode of administration and the solubility of the anti-pruritic agent in the selected carrier or vehicle. The effective concentration is sufficient for treating or alleviating pruritus, and can be empirically determined. The concentration is generally greater than the concentration for systemic administration of the compound.

The resulting mixture can be a solution, suspension, emulsion or the like, and can be formulated as a cream, gel, ointment, emulsion, solution, elixir, lotion, suspension, tincture, paste, foam, aerosol, irrigation, spray, suppository, bandage, or any other formulation suitable for topical or local administration. Modes of administration can include topical application to the skin, scalp, eyes, and/or nasal, buccal or sublingual mucosa.

Pharmaceutical and cosmetic carriers or vehicles suitable for administration of the compositions include any such carriers known to those skilled in the art to be suitable for the particular mode of administration. The anti-pruritic agent can be included in the carriers in amounts sufficient to exert a therapeutically useful effect without serious toxic effects on the treated individual.

To formulate these compositions, a weight fraction of an anti-pruritic agent is dissolved, suspended, dispersed or otherwise mixed in a selected vehicle at an effective concentration such that the pruritic condition is relieved or ameliorated. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream (USP), and hydrophilic ointment (USP).

The compositions employed in the present methods can relieve pruritus when applied to the skin. The composition can be administered topically to the affected area up to eight times per day, as needed, to provide reduction in and relief from itching. Relief can be temporary or permanent, and can even be evident after a single dose of the composition. When the composition is administered in a form other than a topical preparation, it should be administered in an amount sufficient to provide relief from pruritus that is within safety guidelines established by the FDA. Determining the appropriate amount to administer to a patient is within the skill of the person of ordinary skill in the art in association with teachings provided by the present invention.

Solutions of the compositions of this invention intended for topical administration contain an amount of the composition effective to deliver an anti-pruritic amount, typically at a concentration of between about 0.01% w/w to about 5% w/w. The balance of the solution is water, a suitable organic solvent or other suitable solvent or buffer. These compositions that are formulated as solutions or suspensions can be applied to the skin, or can be formulated as an aerosol or foam and applied to the skin as a spray-on. The aerosol compositions typically contain from 25% to 80% w/w, preferably from 30% to 50% w/w, of a suitable propellant. Gel compositions can be formulated by simply admixing a suitable thickening agent to the solution or suspension.

Solutions and suspensions can also be topically applied to the eyes and mucosa. Solutions, particularly those intended for ophthalmic use, can be formulated as 0.01%-10% w/w isotonic solutions, pH about 5-7, with appropriate salts, and preferably containing one or more of the compositions herein at a concentration of about 0.1% w/w, up to about 5% w/w or more. Suitable ophthalmic solutions are known in the art.

Compositions of solid forms intended for topical application can be formulated as stick-type compositions intended for application to the lips or other parts of the body. Such compositions contain an effective amount of an anti-pruritic agent, e.g. nalbuphine or a pharmaceutically acceptable salt, solvate or ester thereof. The amount of the anti-pruritic agent present is typically from about 0.01% w/w to about 5% w/w. The solids also contain from about 40% to 98% w/w, preferably from about 50% to 90% w/w, of emollients. This composition can further contain from 1% to 20% w/w, preferably from 5% to 15% w/w, of a suitable thickening agent, and, if desired or needed, emulsifiers and water or buffers.

In addition, the compositions, and preparations containing the compositions, can also be coated on bandages, mixed with bioadhesives, or included in dressings. Thus, combinations of bandages, bioadhesives, dressings and other such materials and the compositions formulated as described herein are provided.

### Sustained Release Formulations

Nalbuphine formulations which can be employed in the present methods include oral sustained release nalbuphine formulations as described in U.S. Provisional Pat. Appl. Nos. 60/772,466, 60/710,772, and 62/011,936; U.S. patent application Ser. No. 11/509,347 (published as US 2007/0048376), Ser. No. 12/154,496 (published as US 2009/0030026), and Ser. No. 14/738,550; and PCT Appl. No. PCT/US2015/035650; each of which is incorporated herein by reference in their entireties.

In some embodiments, the present methods can employ oral sustained release formulations of nalbuphine including an anti-pruritic effective amount of nalbuphine or a pharmaceutically acceptable salt, solvate or ester thereof. The oral sustained release formulations can provide a controlled release of the drug over a longer period than observed for bolus injections or immediate release oral formulations (e.g., at least about 8-12 hours). Reducing the frequency of dosing provides the potential for enhanced patient convenience and compliance with the present methods. The lower dosing frequency also has the potential to provide reduced side effects because the patient may be exposed to lower peak concentrations of drug over time.

The present methods can employ compositions including nalbuphine or a pharmaceutically acceptable salt, solvate or ester thereof and a sustained release delivery system. The sustained release delivery system includes (i) at least one hydrophilic compound, at least one cross-linking agent, and at least one pharmaceutical diluent; (ii) at least one hydrophilic compound, at least one cross-linking agent, at least one pharmaceutical diluent, and at least one cationic cross-linking agent different from the first cross-linking agent; or (iii) at least one hydrophilic compound, at least one cationic cross-linking compound, and at least one pharmaceutical diluent. Alternatively, in other embodiments, the present methods can employ compositions including nalbuphine or a pharmaceutically acceptable salt, solvate or ester thereof and a sustained release delivery system, which may employ a hydrophobic compound in a sustained release system.

The nalbuphine can be homogeneously dispersed in the sustained release delivery system. In some embodiments, the nalbuphine or pharmaceutically acceptable salt, solvate or ester thereof is present in the composition in an amount of about 1 mg to about 240 mg; about 1 mg to about 150 mg; about 1 mg to about 125 mg; or about 1 mg to about 100 mg. In some embodiments, the nalbuphine or pharmaceutically acceptable salt, solvate or ester thereof is present in the composition in an amount of about 5 mg to about 80 mg; about 10 mg to about 70 mg; about 15 mg to about 60 mg; about 40 mg to about 80 mg; about 50 mg to about 70 mg; or about 45 mg to about 60 mg. In one embodiment, the nalbuphine or pharmaceutically acceptable salt, solvate or ester thereof is present in the composition in an amount of about 15 mg, about 20 mg, about 25 mg, about 30 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, or about 240 mg. In another embodiment, the nalbuphine or pharmaceutically acceptable salt thereof is present in the composition in an amount of about 30 mg, about 45 mg, about 60 mg, about 120 mg, or about 180 mg. In yet another embodiment, the nalbuphine or pharmaceutically acceptable salt thereof is present in the composition in an amount of about 15 mg, 30 mg, 90 mg, 120 mg or 180 mg.

In yet another embodiment, the nalbuphine or pharmaceutically acceptable salt thereof, e.g., HCL is present in the composition in an amount of about 15 mg, about 30 mg, about 60 mg, about 90 mg, about 120 mg, or about 180 mg.

In some embodiments, the sustained release delivery system is present in the composition in an amount from about 10 mg to about 420 mg; from about 25 mg to about 225 mg; from about 21 mg to about 198 mg; or from about 80 mg to about 200 mg; from about 80 mg to about 220 mg; from about 90 mg to about 210 mg; from about 100 mg to about 200 mg; from about 110 mg to about 190 mg; from about 120 mg to about 180 mg; from about 130 mg to about 170 mg; from about 140 mg to about 160 mg; from about 30 mg to about 60 mg; from about 60 mg to about 180 mg; from about 30 mg to about 180 mg, from about 75 mg to about 150 mg, from about 80 mg to about 160 mg, from about 90 mg to about 150 mg, from about 100 mg to about 140 mg, from about 110 mg to about 130 mg, from about 100 mg to about 300 mg, from about 200 mg to about 300 mg or from about 200 mg to about 250 mg. In one embodiment, the sustained release delivery system is present in the composition in an amount from about 75 mg to about 150 mg.

In some embodiments, the sustained release delivery system is present in the composition in an amount of about 30 mg, about 60 mg, about 75 mg, about 80 mg, about 90 mg, about 100 mg, about 110 mg, about 112 mg, about 115 mg, about 117 mg, about 120 mg, about 125 mg, about 130 mg, about 135 mg, about 140 mg, about 145 mg, about 150 mg, about 160 mg, about 170 mg, about 180 mg, about 190 mg, about 200 mg, about 210 mg, about 220 mg, about 225 mg, about 230 mg, about 240 mg, about 250 mg, about 260 mg, about 270 mg, about 280 mg, about 300 mg, about 320 mg, about 340 mg, about 360 mg, about 380 mg, about 400 mg or about 420 mg. In another embodiment, the sustained release delivery system is present in the composition in an amount of about 112 mg.

The ratio of nalbuphine or pharmaceutically acceptable salt, solvate or ester thereof in the compositions to the sustained release delivery system is generally from about 4:1 to about 1:25. In some embodiments, the ratio of nalbuphine or pharmaceutically acceptable salt, solvate or ester thereof to the sustained release delivery system is generally from about 2.5:1 to about 1:4. In some embodiments, the ratio of nalbuphine or pharmaceutically acceptable salt, solvate or ester thereof to the sustained release delivery system is generally from about 5:1 to about 1:5, about 4:1 to about 1:4, about 3:1 to about 1:3, about 2:1 to about 1:2, about 1:1 to about 1:5, about 1:1 to about 1:4, about 1:1 to about 1:3, about 1:1 to about 1.2, and about 1:2 to about 1:3. In some embodiments, the ratio of nalbuphine or pharmaceutically acceptable salt, solvate or ester thereof to the sustained release delivery system is about 1:1, about 1:2, about 1:2.5, about 1:3, about 1:4, or about 1:5.

In one embodiment, at least one hydrophilic compound is present in the sustained release delivery system in an amount of about 5% to about 80% by weight; the at least one cross-linking agent is present in the sustained release delivery system in an amount of about 0.5% to about 80% by weight; and the at least one pharmaceutical diluent is present in the sustained release delivery system in an amount of about 20% to about 80% by weight. In another embodiment, the at least one hydrophilic compound is present in the sustained release delivery system in an amount of about 8% to about 31% by weight; the at least one cross-linking agent is present in the sustained release delivery system in an amount of about 12% to about 47% by weight; and the at least one pharmaceutical diluent is present in the sustained release delivery system in an amount of about 20% to about 78% by weight. In another embodiment, the at least one hydrophilic compound is present in the sustained release delivery system in an amount of about 10% to about 20% by weight; the at least one cross-linking agent is present in the sustained release delivery system in an amount of about 15% to about 25% by weight; and the at least one pharmaceutical diluent is present in the sustained release delivery system in an amount of about 50% to about 85% by weight. In some embodiments, the at least one hydrophilic compound is present in the sustained release delivery system in an amount of about 8%, about 9%, about 10%, about 11%, about 12%, about 13%, about 14%, about 15%, about 16%, about 17%, about 18%, about 19%, about 20%, about 22%, about 24%, about 26%, about 28%, about 30%, about 32%, about 34%, or about 36% by weight; the at least one cross-linking agent is present in the sustained release delivery system in an amount of about 10%, about 11%, about 12%, about 13%, about 14%, about 15%, about 16%, about 17%, about 18%, about 19%, about 20%, about 22%, about 24%, about 26%, about 28%, about 30%, about 32%, about 33%, about 34%, or about 35% by weight; and the at least one pharmaceutical diluent is present in the sustained release delivery system in an amount of about 40%, about 45%, about 50%, about 55%, about 60%, about 65%, about 70%, about 80%, or about 85% by weight.

In some embodiments, the at least one hydrophilic compound is present in the sustained release delivery system in an amount of about 10%, about 11%, about 12%, about 13%, about 14%, about 15%, about 16%, about 17%, about 18%, about 19%, or about 20% by weight; the at least one cross-linking agent is present in the sustained release delivery system in an amount of about 15%, about 16%, about 17%, about 18%, about 19%, about 20%, or about 22% by weight; and the at least one pharmaceutical diluent is present in the sustained release delivery system in an amount of about 55%, about 60%, about 65%, about 70%, about 80%, or about 85% by weight. In one embodiment, the at least one hydrophilic compound is present in the sustained release delivery system in an amount of about 8%, about 12%, or about 20% by weight; the at least one cross-linking agent is present in the sustained release delivery system in an amount of about 12%, about 18%, or about 30% by weight; and the at least one pharmaceutical diluent is present in the sustained release delivery system in an amount of about 40%, about 60%, or about 70% by weight.

In one embodiment, nalbuphine is in the form of any pharmaceutically acceptable salt known in the art. Exemplary pharmaceutically acceptable salts include without limitation hydrochloric, sulfuric, nitric, phosphoric, hydrobromic, maleic, malic, ascorbic, citric, tartaric, pamoic, lauric, stearic, palmitic, oleic, myristic, lauryl sulfuric, napthalenesulfonic, linoleic, linolenic acid, and the like. One embodiment includes the hydrochloride salt of nalbuphine.

The sustained release delivery system includes at least one hydrophilic compound. The hydrophilic compound preferably forms a gel matrix that releases the nalbuphine or the pharmaceutically acceptable salt, solvate or ester thereof at a sustained rate upon exposure to liquids. The rate of release of the nalbuphine or the pharmaceutically acceptable salt, solvate or ester thereof from the gel matrix depends on the drug's partition coefficient between the components of the gel matrix and the aqueous phase within the gastrointestinal tract. The weight ratio of nalbuphine to hydrophilic compound is generally in the range of about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, and about 2:1 to about 1:2. In some embodiments, the weight ratio of nalbuphine to hydrophilic compound is in the range of about 10:1 to about 1:1, about 10:1 to about 2:1, about 9:1 to about 1:1, about 8:1 to about 1:1, about 7:1 to about 1:1, about 6:1 to about 1:1, about 5:1 to about 1:1, about 4:1 to about 1:1, about 3:1 to about 1:1, and about 2:1 to about 1:1. In some embodiments, the weight ratio of nalbuphine to hydrophilic compound is in the range of about 6:1 to about 1:1, about 5:1 to about 2:1, about 4:1 to about 3:1, about 4:1 to about 2:1, and about 5:1 to about 2:1. In some embodiments, the weight ratio of nalbuphine to hydrophilic compound is about 1:5, about 1:4.5, about 1:4.4, about 1:4, about 1:3.5, about 1:3.3, about 1:3, about 1:2.5, about 1:2, about 1:1, and about 1:1.5.

The sustained release delivery system generally includes the hydrophilic compound in an amount of about 5% to about 80% by weight. In some embodiments, the sustained release delivery system generally includes the hydrophilic compound in an amount of about 5% to about 30%, about 8% to about 31%, about 10% to about 20%, about 20% to about 60%, or about 40% to about 60% by weight. In one embodiment, the sustained release delivery system includes the hydrophilic compound in an amount of about 8% to about 31% by weight. In one embodiment, the sustained release delivery system includes the hydrophilic compound in an amount of about 10% to about 20% by weight. In some embodiments, the sustained release delivery system includes the hydrophilic compound in an amount of about 10%, about 11%, about 12%, about 13%, about 14%, about 15%, about 16%, about 17%, about 18%, about 19%, or about 20% by weight. In one embodiment, the sustained release delivery system includes the hydrophilic compound in an amount of about 12% by weight. In one embodiment, the sustained release delivery system includes the hydrophilic compound in an amount of about 8% by weight. In one embodiment, the sustained release delivery system includes the hydrophilic compound in an amount of about 20% by weight. In one embodiment, the sustained release delivery system includes the hydrophilic compound in an amount of about 28% by weight.

The hydrophilic compound is any compound known in the art to be hydrophilic. Exemplary hydrophilic compounds include without limitation gums, cellulose ethers, polyvinyl pyrrolidone, protein-derived compounds, and mixtures thereof. Exemplary gums include without limitation heteropolysaccharide gums and homopolysaccharide gums, such as xanthan, tragacanth, pectins, acacia, karaya, alginates, agar, guar, hydroxypropyl guar, carrageenan, locust bean gums, and gellan gums. Exemplary cellulose ethers include without limitation hydroxyalkyl celluloses and carboxyalkyl celluloses. In some embodiments, cellulose ethers include hydroxyethyl celluloses, hydroxypropyl celluloses, hydroxypropylmethyl-celluloses, carboxy methylcelluloses, and mixtures thereof. In some embodiments, the hydrophilic compound is a gum. In other embodiments, the hydrophilic compound is a heteropolysaccharide gum. In further embodiments, the hydrophilic compound is a xanthan gum or derivative thereof. Derivatives of xanthan gum include without limitation, for example, deacylated xanthan gum, the carboxymethyl esters of xanthan gum, and the propylene glycol esters of xanthan gum.

In another aspect, the sustained release delivery system further includes at least one cross-linking agent. In one embodiment, the cross-linking agent is a compound that is capable of cross-linking the hydrophilic compound to form a gel matrix in the presence of liquids. As used herein, “liquids” includes, for example, gastrointestinal fluids and aqueous solutions, such as those used for in vitro dissolution testing. The sustained release delivery system generally includes the cross-linking agent in an amount of about 0.5% to about 80% by weight. In one embodiment, the sustained release delivery system generally includes the cross-linking agent in an amount of about 12% to about 47% by weight. In another embodiment, the sustained release delivery system generally includes the cross-linking agent in an amount of about 20% to about 30% by weight. In one embodiment, the sustained release delivery system generally includes the cross-linking agent in an amount of about 15% to about 25% by weight. In some embodiments, the at least one cross-linking agent is present in the sustained release delivery system in an amount of about 15%, about 16%, about 17%, about 18%, about 19%, about 20%, about 21%, about 22%, about 23%, about 24%, or about 25% by weight. In one embodiment, the sustained release delivery system includes the cross-linking agent in an amount of about 18% by weight. In one embodiment, the sustained release delivery system includes the cross-linking agent in an amount of about 12% by weight. In one embodiment, the sustained release delivery system includes the cross-linking agent in an amount of about 30% by weight. In one embodiment, the sustained release delivery system includes the cross-linking agent in an amount of about 42% by weight.

Exemplary cross-linking agents include homopolysaccharides. Exemplary homopolysaccharides include without limitation galactomannan gums, such as guar gum, hydroxypropyl guar gum, and locust bean gum. In some embodiments, the cross-linking agent is a locust bean gum or a guar gum. In other embodiments, the cross-linking agent is an alginic acid derivative or hydrocolloid.

In some embodiments, when the sustained release delivery system includes at least one hydrophilic compound and at least one cross-linking agent, the weight ratio of hydrophilic compound to cross-linking agent is from about 1:9 to about 9:1, about 1:8 to about 8:1, about 1:7 to about 7:1, about 1:6 to about 6:1, about 1:5 to about 5:1, about 1:4 to about 4:1, about 1:3 to about 3:1, or about 1:2 to about 2:1. In some embodiments, the weight ratio of hydrophilic compound to cross-linking agent is about 1:5, about 1:4.5, about 1:4, about 1:3.5, about 1:3, about 1:2.5, about 1:2, about 1:1.5, and about 1:1.

When the sustained release delivery system includes at least one hydrophilic compound and at least one cross-linking agent, the weight ratio of the nalbuphine or pharmaceutically acceptable salt, solvate or ester thereof to the sum of the at least one hydrophilic compound and the at least one cross-linking agent is from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, or from about 2:1 to about 1:2. In some embodiments, the weight ratio of the nalbuphine or pharmaceutically acceptable salt, solvate or ester thereof to the sum of the at least one hydrophilic compound and the at least one cross-linking agent is from about 4:1 to about 1:1, from about 4:1 to about 1:1.5, from about 3:1 to about 1:1, or from about 2:1 to about 1:1. In one embodiment, the ratio of the nalbuphine or pharmaceutically acceptable salt, solvate or ester thereof to the sum of the at least one hydrophilic compound and the at least one cross-linking agent is about 5:1, about 4:1 (i.e., 1:0.25), about 3.5:1, about 3:1, about 2.5:1, about 2:1 (i.e., 1:0.5), about 1.9:1, about 1.8:1, about 1.7:1, about 1.6:1, about 1.5:1, about 1.4:1, about 1.3:1, about 1.2:1, about 1.1:1, about 1:1, about 1:1.5, about 1:2, about 1:3, about 1:4, and about 1:5.

The sustained release delivery system further includes one or more pharmaceutical diluents known in the art. Exemplary pharmaceutical diluents include without limitation monosaccharides, disaccharides, polyhydric alcohols and mixtures thereof. In some embodiments, pharmaceutical diluents include, for example, starch, mannitol, lactose, dextrose, sucrose, microcrystalline cellulose, sorbitol, xylitol, fructose, and mixtures thereof. In some embodiments, the pharmaceutical diluent is water-soluble. Nonlimiting examples of water-soluble pharmaceutical diluents include lactose, dextrose, sucrose, or mixtures thereof. The weight ratio of pharmaceutical diluent to hydrophilic compound is generally from about 1:9 to about 9:1, from about 1:8 to about 8:1, from about 1:7 to about 7:1, from about 1:6 to about 6:1, from about 1:5 to about 5:1, from about 1:4 to about 4:1, from about 1:3 to about 3:1, or from about 1:2 to about 2:1. In some embodiments, the weight ratio of pharmaceutical diluent to hydrophilic compound is generally from about 9:1 to about 1:1.5. In some embodiments, the weight ratio of pharmaceutical diluent to hydrophilic compound is about 9:1, about 8.75:1, about 8.5:1, about 8.25:1, about 8:1, about 7.5:1, about 7:1, about 6.5:1, about 6:1, about 5.5:1, about 5:1, about 4.5:1, about 4:1, about 3.5:1, about 3:1, about 2.5:1, about 2:1, about 1.5:1, or about 1:1.

The sustained release delivery system generally includes one or more pharmaceutical diluents in an amount of about 20% to about 80%, about 30% to about 70%, about 40% to about 70%, or about 40% to about 60%. In one embodiment, the sustained release delivery system includes one or more pharmaceutical diluents in an amount of about 20% to about 70% by weight. In one embodiment, the sustained release delivery system includes one or more pharmaceutical diluents in an amount of about 50% to about 85% by weight. In some embodiments, the sustained release delivery system includes one or more pharmaceutical diluents in an amount of about 55%, about 60%, about 65%, about 70%, about 80%, or about 85% by weight. In one embodiment, the sustained release delivery system includes one or more pharmaceutical diluents in an amount of about 20% by weight. In one embodiment, the sustained release delivery system includes one or more pharmaceutical diluents in an amount of about 30% by weight. In one embodiment, the sustained release delivery system includes one or more pharmaceutical diluents in an amount of about 40% by weight. In one embodiment, the sustained release delivery system includes one or more pharmaceutical diluents in an amount of about 50% by weight. In one embodiment, the sustained release delivery system includes one or more pharmaceutical diluents in an amount of about 60% by weight. In one embodiment, the sustained release delivery system includes one or more pharmaceutical diluents in an amount of about 70% by weight.

In a further aspect, the sustained release delivery system includes one or more cationic cross-linking compounds. In some embodiments, the one or more cationic cross-linking compounds are used instead of the cross-linking agent. In some embodiments, the one or more cationic cross-linking compounds are used in addition to the cross-linking agent. In one embodiment, the one or more cationic cross-linking compounds are used in an amount sufficient to cross-link the hydrophilic compound to form a gel matrix in the presence of liquids. In some embodiments, the one or more cationic cross-linking compounds are present in the sustained release delivery system in an amount of about 0.5% to about 30%, about 0.5% to about 25%, about 0.5% to about 20%, about 0.5% to about 15%, about 0.5% to about 10%, or about 0.5% to about 5% by weight. In some embodiments, the one or more cationic cross-linking compounds are present in the sustained release delivery system in an amount of about 5% to about 20%, about 5% to about 15%, about 6% to about 14%, about 7% to about 13%, about 8% to about 12%, or about 9% to about 11% by weight. In some embodiments, the one or more cationic cross-linking compounds are present in the sustained release delivery system in an amount of about 5%, about 6%, about 7%, about 8%, about 9%, about 10%, about 11%, about 12%, about 13%, about 14%, or about 15% by weight. In one embodiment, the cationic cross-linking compound is present in the sustained release delivery system in an amount of about 10% by weight.

Exemplary cationic cross-linking compounds include without limitation monovalent metal cations, multivalent metal cations, and inorganic salts, including alkali metal and/or alkaline earth metal sulfates, chlorides, borates, bromides, citrates, acetates, lactates, and mixtures thereof. For example, the cationic cross-linking compound include without limitation one or more of calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, or mixtures thereof.

When the sustained release delivery system includes at least one hydrophilic compound and at least one cationic cross-linking compound, the weight ratio of hydrophilic compound to cationic cross-linking compound ranges from about 1:9 to about 9:1, from about 1:8 to about 8:1, from about 1:7 to about 7:1, from about 1:6 to about 6:1, from about 1:5 to about 5:1, from about 1:4 to about 4:1, from about 1:3 to about 3:1, or from about 1:2 to about 2:1. In one embodiment, the weight ratio of hydrophilic compound to cationic cross-linking compound ranges from about 1:3 to about 3:1. In some embodiments, the weight ratio of hydrophilic compound to cationic cross-linking compound is about 3:1, about 2.75:1, about 2.5:1, about 2.25:1, about 2:1, about 1.8:1, about 1.6:1, about 1.4:1, about 1.2:1, about 1:1, about 1:1.25, about 1:1.5, or about 1:2. In one embodiment, the weight ratio of hydrophilic compound to cationic cross-linking compound is about 1:1.25. In one embodiment, the weight ratio of hydrophilic compound to cationic cross-linking compound is about 1.2:1. In one embodiment, the weight ratio of hydrophilic compound to cationic cross-linking compound is about 2:1. In one embodiment, the weight ratio of hydrophilic compound to cationic cross-linking compound is about 2.8:1.

In one embodiment, the at least one hydrophilic compound is present in the sustained release delivery system in an amount of about 5% to about 80% by weight; the at least one cationic cross-linking agent is present in the sustained release delivery system in an amount of about 0.5% to about 30% by weight; and the at least one pharmaceutical diluent is present in the sustained release delivery system in an amount of about 20% to about 80% by weight. In another embodiment, the at least one hydrophilic compound is present in the sustained release delivery system in an amount of about 8% to about 30% by weight; the at least one cationic cross-linking agent is present in the sustained release delivery system in an amount of about 10% by weight; and the at least one pharmaceutical diluent is present in the sustained release delivery system in an amount of about 20% to about 70% by weight. In another embodiment, the at least one hydrophilic compound is present in the sustained release delivery system in an amount of about 5% to about 30% by weight; the at least one cationic cross-linking agent is present in the sustained release delivery system in an amount of about 5% to about 20% by weight; and the at least one pharmaceutical diluent is present in the sustained release delivery system in an amount of about 20% to about 85% by weight. In another embodiment, the at least one hydrophilic compound is present in the sustained release delivery system in an amount of about 10% to about 20% by weight; the at least one cationic cross-linking agent is present in the sustained release delivery system in an amount of about 5% to about 15% by weight; and the at least one pharmaceutical diluent is present in the sustained release delivery system in an amount of about 50% to about 85% by weight.

In some embodiments, the at least one hydrophilic compound is present in the sustained release delivery system in an amount of about 8%, about 9%, about 10%, about 11%, about 12%, about 13%, about 14%, about 15%, about 16%, about 17%, about 18%, about 19%, about 20%, about 22%, about 24%, about 26%, about 28%, or about 30% by weight; the at least one cationic cross-linking agent is present in the sustained release delivery system in an amount of about 5%, about 6%, about 7%, about 8%, about 9%, about 10%, about 11%, about 12%, about 13%, about 14%, about 15%, about 16%, about 17%, about 18%, about 19%, or about 20%, by weight; and the at least one pharmaceutical diluent is present in the sustained release delivery system in an amount of about 40%, about 45%, about 50%, about 55%, about 60%, about 65%, about 70%, about 80%, or about 85% by weight. In one embodiment, the at least one hydrophilic compound is present in the sustained release delivery system in an amount of about 10%, about 11%, about 12%, about 13%, about 14%, about 15%, about 16%, about 17%, about 18%, about 19%, or about 20% by weight; the at least one cationic cross-linking agent is present in the sustained release delivery system in an amount of about 5%, about 6%, about 7%, about 8%, about 9%, about 10%, about 11%, about 12%, about 13%, about 14%, about 15%, by weight; and the at least one pharmaceutical diluent is present in the sustained release delivery system in an amount of about 55%, about 60%, about 65%, about 70%, about 80%, or about 85% by weight. In one embodiment, the at least one hydrophilic compound is present in the sustained release delivery system in an amount of about 8%, about 12%, or about 20% by weight; the at least one cationic cross-linking agent is present in the sustained release delivery system in an amount of about 10%, about 12%, or about 14% by weight; and the at least one pharmaceutical diluent is present in the sustained release delivery system in an amount of about 40%, about 60%, or about 70% by weight.

In one embodiment, the sustained release delivery system includes about 0.5% to about 80% locust bean gum, about 5% to about 80% xanthan gum, about 20% to about 80% mannitol and about 0.5% to 80% calcium sulfate dihydrate. In one embodiment, the sustained release delivery system includes about 12% to about 47% locust bean gum, about 8% to about 31% xanthan gum, about 20% to about 78% mannitol and about 0.5% to 25% calcium sulfate dihydrate. In one embodiment, the sustained release delivery system includes about 15% to about 25% locust bean gum, about 10% to about 20% xanthan gum, about 50% to about 85% mannitol and about 5% to 15% calcium sulfate dihydrate. In one embodiment, the sustained release delivery system includes about 18% locust bean gum, about 12% xanthan gum, about 60% mannitol and about 10% calcium sulfate dihydrate. In one embodiment, the sustained release delivery system includes about 12% locust bean gum, about 8% xanthan gum, about 70% mannitol and about 10% calcium sulfate dihydrate. In one embodiment, the sustained release delivery system includes about 20% locust bean gum, about 30% xanthan gum, about 40% mannitol and about 10% calcium sulfate dihydrate. In one embodiment, the sustained release delivery system includes about 30% locust bean gum, about 20% xanthan gum, about 40% mannitol and about 10% calcium sulfate dihydrate. In one embodiment, the sustained release delivery system includes about 42% locust bean gum, about 28% xanthan gum, about 20% mannitol and about 10% calcium sulfate dihydrate.

Two properties of the components of this sustained release system (e.g., the at least one hydrophilic compound and the at least one cross-linking agent; or the at least one hydrophilic compound and at least one cationic cross-linking compound) are that it forms a gel matrix upon exposure to liquids are fast hydration of the compounds/agents and the ability to form a gel matrix having a high gel strength. These two properties, which are needed to achieve a slow release gel matrix, are maximized by the particular combination of compounds (e.g., the at least one hydrophilic compound and the at least one cross-linking agent; or the at least one hydrophilic compound and the at least one cationic cross-linking compound). For example, hydrophilic compounds (e.g., xanthan gum) have excellent water-wicking properties that provide fast hydration. The combination of hydrophilic compounds with materials that are capable of cross-linking the rigid helical ordered structure of the hydrophilic compound (e.g., cross-linking agents and/or cationic cross-linking compounds) thereby acts synergistically to provide a higher than expected viscosity (i.e., high gel strength) of the gel matrix.

In some embodiments, the sustained release compositions are further admixed with one or more wetting agents (e.g., polyethoxylated castor oil, polyethoxylated hydrogenated castor oil, polyethoxylated fatty acid from castor oil, polyethoxylated fatty acid from hydrogenated castor oil) one or more lubricants (e.g., magnesium stearate, sodium stearyl fumarate, and the like), one or more buffering agents, one or more colorants, and/or other conventional ingredients.

In some embodiments compositions employed in the present methods can contain additional pharmaceutical excipients. For example, in certain embodiments, fumaric acid can be added to the formulations described herein.

In other embodiments, a non-functional coating, e.g., Opadry®, can be added to the compositions described herein.

In some embodiments, the compositions described herein further include a second hydrophilic compound. In some embodiments, the second hydrophilic compound is a cellulose ether. In some embodiments, the second hydrophilic compound is a hydroxyalkyl cellulose or a carboxyalkyl cellulose. In some embodiments, the second hydrophilic compound is a hydroxyethyl cellulose, a hydroxypropyl cellulose, a hydroxypropylmethyl-cellulose, a carboxy methylcellulose, or a mixture thereof. In some embodiments, the second hydrophilic is an ethyl cellulose or wax (e.g., including without limitation cetyl alcohol, stearyl alcohol, white wax, or carnauba wax). The second hydrophilic compound is present in the formulation in an amount ranging from about 5% to about 45%, about 5% to about 25%, about 10% to about 20%, or 12% to about 18% by weight. In some embodiments, the second hydrophilic compound is present in the formulation in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, or about 45%.

In some embodiments, the weight ratio of the second hydrophilic compound to the nalbuphine or pharmaceutically acceptable salt, solvate or ester ranges from about 5:1 to about 1:5, about 4:1 to about 1:4, about 3:1 to about 1:3, about 2:1 to about 1:2, about 1:1 to about 1:3, or about 1:1 to about 1:2. In some embodiments, the weight ratio of the second hydrophilic compound to the nalbuphine or pharmaceutically acceptable salt, solvate or ester is about 5:1, about 4:1, about 3:1, about 2:1, about 1:1, about 1:2, about 1:3, about 1:4, or about 1:5.

In some embodiments, the weight ratio of the second hydrophilic compound to the sustained release delivery system ranges from about 10:1 to about 1:10, about 8:1 to about 1:8, about 6:1 to about 1:6, about 4:1 to about 1:4, about 2:1 to about 1:3, about 1:1 to about 1:10, about 1:1 to about 1:6, or about 1:2 to about 1:6. In some embodiments, the weight ratio of the second hydrophilic compound to the sustained release delivery system is about 10:1, about 8:1, about 6:1, about 4:1, about 2:1, about 1:1, about 1:1.5, about 1:2, about 1:2.5, about 1:3, about 1:4, about 1:5, about 1:6, about 1:7, about 1:8, about 1:9 or about 1:10.

In some embodiments, the oral sustained release solid dosage formulations including from about 1 mg to 200 mg nalbuphine hydrochloride and about 10 mg to about 420 mg of a sustained release delivery system. In these embodiments, the sustained release delivery system includes about 12% to about 42% locust bean gum; about 8.0% to about 28% xanthan gum; about 20% to about 70% mannitol; and about 5% to about 20% calcium sulfate dihydrate. In some embodiments, the present methods can employ oral sustained release solid dosage formulations including from about 5 mg to about 80 mg nalbuphine hydrochloride and about 80 mg to about 360 mg of a sustained release delivery system. In some embodiments, the present methods can employ oral sustained release solid dosage formulations including from about 50 mg to about 150 mg nalbuphine hydrochloride and about 100 mg to about 300 mg of a sustained release delivery system.

In some embodiments, the present methods employ oral sustained release solid dosage formulations including about 15 mg nalbuphine hydrochloride, and from about 25 mg to about 225 mg, for example about 195 mg, of a sustained release delivery system. In these embodiments, the sustained release delivery system includes about 14% locust bean gum; about 9% xanthan gum; about 47% mannitol; and about 8% calcium sulfate dihydrate.

In some embodiments, the present methods employ oral sustained release solid dosage formulations including about 30 mg nalbuphine hydrochloride, and from about 25 mg to about 225 mg, for example about 180 mg, of a sustained release delivery system. In these embodiments, the sustained release delivery system includes about 18% locust bean gum; about 12% xanthan gum; about 60% mannitol; and about 10% calcium sulfate dihydrate.

In some embodiments, the present methods employ oral sustained release solid dosage formulations including about 60 mg nalbuphine hydrochloride, and from about 25 mg to about 225 mg, for example about 120 mg, of a sustained release delivery system. In these embodiments, the sustained release delivery system includes about 10% locust bean gum; about 12% xanthan gum; about 60% mannitol; and about 10% calcium sulfate dihydrate. In some embodiments, the present methods employ oral sustained release solid dosage formulations including from about 5 mg to about 80 mg nalbuphine hydrochloride and about 80 mg to about 360 mg of a sustained release delivery system.

In some embodiments, the present methods employ oral sustained release solid dosage formulations including about 120 mg nalbuphine hydrochloride, and from about 25 mg to about 250 mg, for example about 240 mg, of a sustained release delivery system. In these embodiments, the sustained release delivery system includes about 18% locust bean gum; about 12% xanthan gum; about 60% mannitol; and about 10% calcium sulfate dihydrate.

In some embodiments, the present methods employ oral sustained release solid dosage formulations including about 30 mg nalbuphine hydrochloride, and from about 25 mg to about 350 mg, for example about 270 mg or about 360 mg, of a sustained release delivery system. In these embodiments, the sustained release delivery system includes about 18% locust bean gum; about 12% xanthan gum; about 60% mannitol; and about 10% calcium sulfate dihydrate.

In some embodiments, the present methods employ oral sustained release solid dosage formulations including about 45 to about 60 mg nalbuphine hydrochloride and from about 100 mg to about 200 mg of a sustained release delivery system. In these embodiments, the sustained release delivery system includes about 15% to about 25% locust bean gum; about 10% to about 20% xanthan gum; about 50% to about 85% mannitol; and about 5% to about 15% calcium sulfate dihydrate.

In some embodiments, the present methods employ oral sustained release solid dosage formulations including about 30 mg nalbuphine hydrochloride, about 32.4 mg locust bean gum; about 21.6 mg xanthan gum; about 108 mg mannitol; about 18 mg calcium sulfate dihydrate, about 35 mg hydroxypropylcellulose, and about 1.9 mg magnesium stearate.

In some embodiments, the present methods employ oral sustained release solid dosage formulations including about 60 mg nalbuphine hydrochloride, about 21.6 mg locust bean gum; about 14.4 mg xanthan gum; about 72 mg mannitol; about 12 mg calcium sulfate dihydrate, about 30 mg hydroxypropylcellulose, and about 1.6 mg magnesium stearate.

In some embodiments, the present methods employ oral sustained release solid dosage formulations including about 120 mg nalbuphine hydrochloride, about 43.2 mg locust bean gum; about 28.8 mg xanthan gum; about 144 mg mannitol; about 24 mg calcium sulfate dihydrate, about 60 mg hydroxypropylcellulose, and about 3.2 mg magnesium stearate.

In some embodiments, the present methods employ oral sustained release solid dosage formulations including about 180 mg nalbuphine hydrochloride, about 64.8 mg locust bean gum; about 43.2 mg xanthan gum; about 216 mg mannitol; about 36 mg calcium sulfate dihydrate, about 90 mg hydroxypropylcellulose, about 5 mg magnesium stearate, and about 25 mg fumaric acid.

In some embodiments, the present methods employ oral sustained release solid dosage formulations including about 180 mg nalbuphine hydrochloride, about 48.6 mg locust bean gum; about 32.4 mg xanthan gum; about 162 mg mannitol; about 27 mg calcium sulfate dihydrate, about 60 mg hydroxypropylcellulose, about 4 mg magnesium stearate, and about 25 mg fumaric acid.

In some embodiments, the present methods employ oral sustained release solid dosage formulations including about 30 mg nalbuphine hydrochloride, about 32.4 mg locust bean gum; about 21.6 mg xanthan gum; about 108 mg mannitol; about 18 mg calcium sulfate dihydrate, about 35 mg hydroxypropylcellulose, about 1.9 mg magnesium stearate, and about 7.4 mg Opadry II White.

The sustained release formulations of nalbuphine are orally administrable solid dosage formulations. Nonlimiting examples of oral solid dosage formulations include tablets, capsules including a plurality of granules, sublingual tablets, powders, granules, syrups, and buccal dosage forms or devices (e.g., buccal patches, tablets, etc.). In some embodiments, tablets have an enteric coating or a hydrophilic coating.

The sustained release delivery system is prepared by dry granulation or wet granulation, before the nalbuphine or pharmaceutically acceptable salt, solvate or ester thereof is added, although the components can be held together by an agglomeration technique to produce an acceptable product. In the wet granulation technique, the components (e.g., hydrophilic compounds, cross-linking agents, pharmaceutical diluents, cationic cross-linking compounds, hydrophobic polymers, etc.) are mixed together and then moistened with one or more liquids (e.g., water, propylene glycol, glycerol, alcohol) to produce a moistened mass that is subsequently dried. The dried mass is then milled with conventional equipment into granules of the sustained release delivery system. Thereafter, the sustained release delivery system is mixed in the desired amounts with the nalbuphine or the pharmaceutically acceptable salt, solvate or ester thereof and, optionally, one or more wetting agents, one or more lubricants, one or more buffering agents, one or more coloring agents, one or more second hydrophilic compounds, or other conventional ingredients, to produce a granulated composition. The sustained release delivery system and the nalbuphine can be blended with, for example, a high shear mixer. The nalbuphine is preferably finely and homogeneously dispersed in the sustained release delivery system. The granulated composition, in an amount sufficient to make a uniform batch of tablets, is subjected to tableting in a conventional production scale tableting machine at typical compression pressures, i.e., about 2,000-16,000 psi. In some embodiments, the mixture should not be compressed to a point where there is subsequent difficulty with hydration upon exposure to liquids.

In some embodiments, the nalbuphine formulation is prepared by dry granulation or wet granulation. The components of the sustained release delivery system are added, along with the nalbuphine or a pharmaceutically acceptable salt, solvate or ester thereof. Alternatively, all of the components can be held together by an agglomeration technique to produce an acceptable product. In the wet granulation technique, nalbuphine or pharmaceutically salt, solvate or ester thereof and the components (e.g., hydrophilic compounds, cross-linking agents, pharmaceutical diluents, cationic cross-linking compounds, hydrophobic polymers, etc.) are mixed together and then moistened with one or more liquids (e.g., water, propylene glycol, glycerol, alcohol) to produce a moistened mass that is subsequently dried. The dried mass is then milled with conventional equipment into granules. Optionally, one or more wetting agents, one or more lubricants, one or more buffering agents, one or more coloring agents, one or more second hydrophilic compounds, or other conventional ingredients, are also added to the granulation. The granulated composition, in an amount sufficient to make a uniform batch of tablets, is subjected to tableting in a conventional production scale tableting machine at typical compression pressures, i.e., about 2,000-16,000 psi. In some embodiments, the mixture should not be compressed to a point where there is subsequent difficulty with hydration upon exposure to liquids.

The average particle size of the granulated composition is from about 50 μm to about 400 gm by weight. In some embodiments, the average particle size by weight is from about 185 μm to about 265 The average density of the granulated composition is from about 0.3 g/mL to about 0.8 g/mL. In some embodiments, the average density is from about 0.5 g/mL to about 0.7 g/mL. The tablets formed from the granulations are generally from about 4 Kp to about 22 Kp hardness. The average flow of the granulations is from about 25 to about 40 g/sec.

In some embodiments, the present methods can employ a multilayer solid dosage form, in which the layers are formulated to release the nalbuphine hydrochloride at different rates. For example, in one embodiment, the second layer is an extended release layer that includes nalbuphine or a pharmaceutically acceptable salt, solvate or ester thereof and a sustained release delivery system designed to release the nalbuphine or the pharmaceutically acceptable salt, solvate or ester thereof at a controlled rate so that therapeutically beneficial blood levels are maintained over an extended period of time (e.g., from about 8 to about 12 hours). The first layer is an immediate release layer that includes a formulation of nalbuphine or a pharmaceutically acceptable salt, solvate or ester thereof designed to release the nalbuphine or the pharmaceutically acceptable salt, solvate or ester thereof at a rate that is faster than the rate of the second layer to achieve a therapeutically beneficial blood level in an immediate period of time (e.g., from about 1 to about 2 hours). In some embodiments, the first layer includes a sustained release delivery system. In some embodiments, the first layer does not include a sustained release delivery system.

In some embodiments, the weight ratio of the second layer to the first layer is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, about 2:1 to about 1:2. In one embodiment, the weight ratio of the second layer to the first layer is about 5:1 to about 1:5. In a further embodiment, the weight ratio of the second layer to the first layer is about 1:1 to about 1:2. In some embodiments, the weight ratio of the second layer to the first layer is about 1:1, about 1:1.2, about 1:1.4, about 1:1.6, about 1:1.8, or about 1:2. In one embodiment, the weight ratio of the second layer to the first layer is about 1:2. In one embodiment, the weight ratio of the second layer to the first layer is about 1:1.4. In some embodiments, the weight ratio of the second layer to the first layer is about 3:1, about 2.5:1, about 2:1, about 1.5:1. In one embodiment, the weight ratio of the second layer to the first layer is about 2.5:1.

The sustained release delivery system of the multilayer dosage form includes (i) at least one hydrophilic compound, at least one cross-linking agent, and at least one pharmaceutical diluent; (ii) at least one hydrophilic compound, at least one cross-linking agent, at least one pharmaceutical diluent, and at least one cationic cross-linking agent different from the first cross-linking agent; or (iii) at least one hydrophilic compound, at least one cationic cross-linking compound, and at least one pharmaceutical diluent. In some embodiments, when the first layer includes a sustained release delivery system, the sustained release delivery system of the first layer includes the same components as the sustained release delivery system of the second layer (e.g., both the first and second layers are one of embodiments (i)-(iii), listed above). In other embodiments, the sustained release delivery system of the first layer includes different components as the sustained release delivery system of the second layer (e.g., the first layer is embodiment (i), listed above, while the second layer is embodiment (iii), listed above). It is recognized that the sustained release delivery system of either layer can be one of embodiments (i)-(iii) listed above. Moreover, it is recognized that in some embodiments, the first layer does not include a sustained release delivery system.

The sustained release delivery system is generally present in the second layer (e.g., extended release layer) in an amount ranging from about 10 mg to about 420 mg. In some embodiments, the sustained release delivery system is present in the second layer in an amount ranging from about 110 mg to about 200 mg. In some embodiments, the sustained release delivery system is present in the second layer in an amount ranging from about 110 mg to about 150 mg. In some embodiments, the sustained release delivery system is present in the second layer in an amount ranging from about 90 mg to about 150 mg. In some embodiments, the sustained release delivery system is present in the second layer in an amount of about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg. In one embodiment, the sustained release delivery system is present in the second layer in an amount of about 123 mg. In one embodiment, the sustained release delivery system is present in the second layer in an amount of about 101 mg. In one embodiment, the sustained release delivery system is present in the second layer in an amount of about 92 mg. In another embodiment, the sustained release delivery system is present in the second layer in an amount of about 112.5 mg. In one embodiment, the sustained release delivery system is present in the second layer in an amount of about 135 mg. In one embodiment, the sustained release delivery system is present in the second layer in an amount of about 150 mg.

Nalbuphine or a pharmaceutically acceptable salt, solvate or ester thereof is generally present in the second layer in an amount ranging from about 15 mg to about 60 mg. In some embodiments, nalbuphine or a pharmaceutically acceptable salt, solvate or ester thereof is present in the second layer in an amount ranging from about 30 mg to about 60 mg. In some embodiments, nalbuphine or a pharmaceutically acceptable salt, solvate or ester thereof is present in the second layer in an amount ranging from about 45 mg to about 60 mg. In one embodiment, nalbuphine or a pharmaceutically acceptable salt, solvate or ester thereof is present in the second layer in an amount of about 15 mg. In one embodiment, nalbuphine or a pharmaceutically acceptable salt, solvate or ester thereof is present in the second layer in an amount of about 30 mg. In one embodiment, nalbuphine or a pharmaceutically acceptable salt, solvate or ester thereof is present in the second layer in an amount of about 45 mg. In one embodiment, nalbuphine or a pharmaceutically acceptable salt, solvate or ester thereof is present in the second layer in an amount of about 15 mg, about 30 mg, about 60 mg, about 90 mg, about 120 mg, or about 180 mg.

In some embodiments, the weight ratio of nalbuphine or a pharmaceutically acceptable salt, solvate or ester thereof to the sustained release delivery system in the second layer is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, or about 2:1 to about 1:2. In one embodiment, the weight ratio of nalbuphine or a pharmaceutically acceptable salt, solvate or ester thereof to the sustained release delivery system in the second layer is about 1:2 to about 1:4. In one embodiment, the weight ratio of nalbuphine or a pharmaceutically acceptable salt, solvate or ester thereof to the sustained release delivery system in the second layer is about 1:1 to about 1:5. In some embodiments, the weight ratio of nalbuphine or a pharmaceutically acceptable salt, solvate or ester thereof to the sustained release delivery system in the second layer is about 1:1, about 1:1.2, about 1:1.4, about 1:1.6, about 1:1.8, about 1:2, about 1:2.5, about 1:3, or about 1:3.5. In one embodiment, the weight ratio of nalbuphine or a pharmaceutically acceptable salt, solvate or ester thereof to the sustained release delivery system in the second layer is about 1:2.5. In another embodiment, the weight ratio of nalbuphine or a pharmaceutically acceptable salt, solvate or ester thereof to the sustained release delivery system in the second layer is about 1:3.3. In a further embodiment, the weight ratio of nalbuphine or a pharmaceutically acceptable salt, solvate or ester thereof to the sustained release delivery system in the second layer is about 1:3. In yet another embodiment, the ratio of nalbuphine or a pharmaceutically acceptable salt, solvate or ester thereof to the sustained release delivery system in the second layer is about 1:2.

When the sustained release delivery system is present in the first layer (e.g., immediate release layer), it is generally present in an amount ranging from about 0 mg to about 50 mg. In some embodiments, the sustained release delivery system is present in the first layer in an amount ranging from about 5 mg to about 25 mg or from about 5 mg to about 15 mg. In one embodiment, the sustained release delivery system is present in the first layer in an amount of about 3 mg to about 9 mg. In one embodiment, the sustained release delivery system is present in the first layer in an amount of about 4 mg to about 6 mg. In some embodiments, the sustained release delivery system is present in the first layer in an amount of about 2 mg, about 4 mg, about 6 mg, about 8 mg, about 10 mg, about 12 mg, about 14 mg, about 15 mg, about 16 mg, about 18 mg, about 20 mg about 25 mg, about 30 mg, about 35 mg, about 40 mg, about 45 mg or about 50 mg. In one embodiment, the sustained release delivery system is present in the first layer in an amount of about 6 mg.

In some embodiments, nalbuphine or a pharmaceutically acceptable salt, solvate or ester thereof is generally present in the first layer (e.g., immediate release layer) in an amount ranging from about 5 mg to about 180 mg. In some embodiments, nalbuphine or a pharmaceutically acceptable salt, solvate or ester thereof is present in the first layer in an amount ranging from about 5 mg to about 25 mg or from about 10 mg to about 20 mg. In some embodiments, the nalbuphine or a pharmaceutically acceptable salt, solvate or ester thereof is present in the first layer in an amount of about 5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg or about 50 mg. In one embodiment, nalbuphine or a pharmaceutically acceptable salt, solvate or ester thereof is present in the first layer in an amount of about 15 mg, about 30 mg, about 60 mg, about 90 mg, about 120 mg, or about 180 mg.

In some embodiments, when the first layer includes a sustained release delivery system, the ratio of nalbuphine or pharmaceutically acceptable salt, solvate or ester thereof to the sustained release delivery system in the first layer is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, about 2:1 to about 1:2. In one embodiment, the ratio of nalbuphine or pharmaceutically acceptable salt, solvate or ester thereof to the sustained release delivery system in the first layer is about 2:1 to about 4:1. In some embodiments, the ratio of nalbuphine or pharmaceutically acceptable salt, solvate or ester thereof to the sustained release delivery system in the first layer is about 5:1, about 4.5:1, about 4:1, about 3.5:1, about 3:1, about 2.5:1, about 2:1, about 1.5:1, or about 1:1. In one embodiment, the ratio of nalbuphine or pharmaceutically acceptable salt, solvate or ester thereof to the sustained release delivery system in the first layer is about 2.5:1. In another embodiment, the ratio of nalbuphine or pharmaceutically acceptable salt, solvate or ester thereof to the sustained release delivery system in the first layer is about 3:1.

In some embodiments, the multilayer dosage form further includes a pharmaceutical disintegrant. The disintegrant promotes the dissolution and absorption of nalbuphine or pharmaceutically acceptable salt, solvate or ester thereof from the immediate release layer. Nonlimiting examples of pharmaceutical disintegrants include croscarmellose sodium, starch glycolate, crospovidone, and unmodified starch. In one embodiment, the disintegrant is in the first layer (i.e., the immediate release layer), of the dosage form. The disintegrant is generally present in the layer in an amount of about 1.5 mg to about 4.5 mg. In one embodiment, the disintegrant is present in an amount of about 3 mg. In one embodiment, the disintegrant is present in the layer in an amount of about 2-10% by weight. In one embodiment, the disintegrant is present in the layer in an amount of about 5% by weight. When the layer contains a sustained release delivery system, the weight ratio of the sustained release delivery system to the disintegrant is in a range of about 5:1 to about 1:5. In some embodiments, the ratio of the sustained release delivery system to the disintegrant is in a range of about 1:1 to about 3:1. In other embodiments, the ratio of the sustained release delivery system to the disintegrant is in a range of about 2:1.

In some embodiments, the multilayer tablets are prepared by first preparing the immediate release layer and extended release layer blends separately. The extended release layer is prepared as described above. The wet granulation of the extended release layer is then dried and milled to an appropriate size. Magnesium stearate is added and mixed with the milled granulation. The immediate release layer is prepared by first mixing the nalbuphine or the pharmaceutically acceptable salt, solvate or ester thereof with one or more diluents (e.g., microcrystalline cellulose). This mix is then optionally mixed with one or more disintegrants. The blend is mixed with magnesium stearate. Finally, the immediate release layer blend and the extended release layer blend are compressed into multi-layer (e.g., bi-layer) tablets.

The invention provides methods for treating pruritus by administering an effective amount, e.g., an effective amount of a sustained release formulation of nalbuphine or a pharmaceutically acceptable salt, solvate or ester thereof to a subject, e.g., human or animal patient in need thereof. An effective amount is an amount sufficient to eliminate or significantly reduce pruritus symptoms or to alleviate those symptoms (e.g., reduce the symptoms, such as itching, compared to the symptoms present prior to administration of the nalbuphine sustained release formulation). “Sustained release” or “extended release” means that the nalbuphine or pharmaceutically acceptable salt, solvate or ester thereof is released from the formulation at a controlled rate so that therapeutically beneficial blood levels (but below toxic levels) of the nalbuphine or pharmaceutically acceptable salt, solvate or ester thereof are maintained over an extended period of time. Alternatively, “sustained release” or “extended release” means that the desired pharmacologic effect is maintained over an extended period of time. Clinical trials of the formulations described herein find that the duration of relief of pruritus symptoms is longer than expected. The half-life of experimental orally administered nalbuphine formulations (i.e., immediate release formulations) has been reported to be relatively short, only about 5-7 hours. Moreover, the published literature suggests that the duration of effect for experimental formulations of immediate release nalbuphine was only about 4 hours. Based on these data, it was expected that a sustained release formulation would provide a duration of anti-pruritic effect for approximately 6-8 hours, i.e., allowing for 2-3 times daily dosing. In the clinical trials described herein, however, nalbuphine sustained release formulations have an anti-pruritic effect of longer than 8 hours. In some cases, the duration of anti-pruritic effect is at least about 12 hours, thus providing the possibility of fewer dosing administrations.

Without wishing to be bound by a particular theory, the longer than expected duration of anti-pruritic effect is attributed to the enterohepatic recirculation of nalbuphine. Nalbuphine forms a glucuronic acid or other type of conjugated metabolite in vivo through enzymatic reaction with an enzyme system such as UDP-glucuronyl transferase. It is also possible that enterohepatic recirculation also occurs when parent drug in the bile is released from the gallbladder into the intestine and reabsorbed. Once formed, the conjugated nalbuphine product is thought to be transported into the gastrointestinal tract via biliary secretion whereby the drug conjugate is cleaved liberating nalbuphine which can be reabsorbed from the intestine. The sustained release formulation can improve the duration of anti-pruritic effect, by more slowly releasing nalbuphine into the in vivo system and allowing more drug to be conjugated and therefore available for recirculation and later reabsorption from the intestine.

In certain embodiments, the chemistry of certain of the components of the formulation, such as the hydrophilic compound (e.g., xanthan gum), is such that the components are considered to be self-buffering agents which are substantially insensitive to the solubility of the nalbuphine and the pH changes along the length of the gastrointestinal tract. Moreover, the chemistry of the components is believed to be similar to certain known muco-adhesive substances, such as polycarbophil. Muco-adhesive properties are desirable for buccal delivery systems. Thus, the sustained release formulation can loosely interact with the mucin in the gastrointestinal tract and thereby provide another mode by which a constant rate of delivery of the nalbuphine is achieved.

The two phenomenon discussed above (buoyancy and muco-adhesive properties) are mechanisms by which the sustained release formulations can interact with the mucin and fluids of the gastrointestinal tract and provide a constant rate of delivery of the nalbuphine.

When measured by USP Procedure Drug Release General Chapter <711> Dissolution, (incorporated by reference herein in its entirety), the sustained release formulations employed in the present methods generally exhibit an in vitro dissolution of about 15% to about 50% by weight nalbuphine after 1 hour, about 45% to about 80% by weight nalbuphine after 4 hours, or at least about 80% by weight nalbuphine after 10 hours. In some embodiments, the in vitro and in vivo release characteristics of the sustained release formulations are modified using mixtures of one or more different water insoluble and/or water soluble compounds, using different plasticizers, varying the thickness of the sustained release film, including providing release-modifying compounds in the coating, and/or by providing passageways through the coating. In some embodiments, the dissolution rate is determined using apparatus USP Type III/250 mL at pH 6.8, 37° C. and 15 dpm. In some embodiments, the dissolution rate is determined using apparatus USP Type III/250 mL performed in pH change (0-1 hours pH 1.2, after hour 1 pH 4.5, after hour 2 pH 6.8) at 37° C. and 15 dpm.

In some embodiments, the sustained release formulation has an in vitro dissolution of about 50% to about 100% by weight nalbuphine after about 6 hours. In some embodiments, the sustained release formulation has an in vitro dissolution of about 75% to about 100% by weight nalbuphine after about 6 hours. In other embodiments, the sustained release formulation has an in vitro dissolution of about 75% to about 100% by weight nalbuphine from about 6 hours to about 8 hours. In further embodiments, the sustained release formulation has an in vitro dissolution of about 80% to about 100% by weight nalbuphine after about 12 hours. In still other embodiments, the sustained release formulation has an in vitro dissolution of about 80% to about 100% by weight nalbuphine from about 12 hours to about 24 hours. In some embodiments, the sustained release formulation has an in vitro dissolution of about 80% to about 100% after about 8 hours to about 12 hours. In yet other embodiments, the sustained release formulation has an in vitro dissolution of about 15% to about 75% by weight nalbuphine after about 1 hour. In still further embodiments, the sustained release formulation has an in vitro dissolution of about 50% by weight nalbuphine after about 1 hour. In some embodiments, the sustained release formulation has an in vitro dissolution of about 50% by weight nalbuphine after about 1 hour and about 75% to about 100% by weight nalbuphine from about 6 hours to about 8 hours. In some embodiments, the sustained release formulation has an in vitro dissolution of about 50% by weight nalbuphine after about 1 hour and about 75% to about 100% by weight nalbuphine from about 8 hours to about 12 hours. In some embodiments, the sustained release formulation has an in vitro dissolution of about 50% by weight nalbuphine after about 1 hour and about 75% to about 100% by weight nalbuphine from about 12 hours to about 24 hours. In some embodiments, the sustained release formulation has an in vitro dissolution of about 50% by weight nalbuphine after about 1 hour and about 80% to about 100% by weight nalbuphine after about 12 hours.

Where the tablet is a multilayer dosage form having a first extended release layer and a second, immediate release, layer, the sustained release formulation has an in vitro dissolution of about 25% to about 75% by weight nalbuphine after about 1 hour. In some embodiments, the multilayer dosage form has an in vitro dissolution of about 25% by weight nalbuphine after about 1 hour. In some embodiments, the multilayer dosage form has an in vitro dissolution of about 50% by weight nalbuphine after about 1 hour. In some embodiments, the multilayer dosage form has an in vitro dissolution of about 75% to about 100% nalbuphine after about 6-8 hours. In some embodiments, the multilayer dosage form has an in vitro dissolution of about 75% to about 100% nalbuphine after about 8-12 hours. In some embodiments, the multilayer dosage form has an in vitro dissolution of about 75% to about 100% nalbuphine after about 12-24 hours. In some embodiments, the multilayer dosage form has an in vitro dissolution of about 75% to about 100% nalbuphine after about 12 hours.

When administered orally to patients having either normal or impaired (e.g., reduced) kidney function, the sustained release formulations described herein exhibit the following in vivo characteristics: (a) a peak plasma level of nalbuphine occurs within about 4 hours to about 6 hours, e.g., for subjects with uremic pruritus or renal impairment, or about 3 hours to about 5 hours, e.g., for subjects without renal impairment after administration; (b) onset of nalbuphine anti-pruritic effect from about 30 minutes of dosing to within about 6 hours of dosing; (c) duration of the nalbuphine anti-pruritic effect is about 2 to about 24 hours; and (d) the relative nalbuphine bioavailability is about 0.5, about 1, about 1.5 or between about 0.5 to about 1.5 compared to an orally administered aqueous solution of nalbuphine. The time of onset for an anti-pruritic effect can depend on at least on dosing and the severity of pruritic symptoms. In some embodiments, the duration of the nalbuphine anti-pruritic effect is at least about 8 hours. In some embodiments, the duration of the nalbuphine anti-pruritic effect is at least about 9 hours. In some embodiments, the duration of the nalbuphine anti-pruritic effect is at least about 10 hours. In some embodiments, the duration of the nalbuphine anti-pruritic effect is at least about 11 hours. In some embodiments, the duration of the nalbuphine anti-pruritic effect is at least about 12 hours. In some embodiments, the duration of nalbuphine anti-pruritic effect is about 6, hours, 8 hours, 10 hours, 12 hours, 15 hours, or 18 hours. In some embodiments, the relative nalbuphine bioavailability is about 0.94 compared to an orally administered aqueous solution of nalbuphine. In some embodiments, the relative nalbuphine bioavailability is about 1.35 compared to an orally administered aqueous solution of nalbuphine.

In some embodiments, the sustained release nalbuphine formulations provide an oral unit dosage form including nalbuphine or a pharmaceutically acceptable salt, solvate or ester thereof. The oral dosage form provides an anti-pruritic effect over a period of at least about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours. In some embodiments, the oral dosage form provides an anti-pruritic effect over a period of about 6-18 hours, about 8-16 hours, about 8-12 hours, about 8 to about 24 hours, about 12 to about 24 hours, about 18 to about 24 hours, or about 8-10 hours. The oral dosage form provides an anti-pruritic effect over a period of about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours.

In one embodiment, the oral dosage form provides an anti-pruritic effect as well as breaking the cycle effect, e.g., the itchy sensation does not return after certain treatment period.

In some embodiments, the oral dosage form provides a blood plasma level of nalbuphine characterized by one or more peaks followed by a plateau region. The plateau region is characterized as having a relatively consistent blood plasma level of nalbuphine (e.g., the blood plasma level of nalbuphine does not consistently increase or decrease from time point to time point). In some embodiments, the plateau region is characterized as having a consistent average blood plasma level of nalbuphine. The plateau region is contrasted with the region following the plateau region, in which the blood plasma level of nalbuphine generally decreases from one time point to the next. In some embodiments, the plateau region has a duration of at least about 1 hour, about 2 hours, about 3 hours, about 4 hours, about 5 hours, about 6 hours, about 7 hours, about 8 hours, about 9 hours, about 10 hours, about 11 hours or about 12 hours. In some embodiments, the plateau region has a duration from about 1 hour to about 12 hours, from about 2 hours to about 10 hours, from about 2 hours to about 8 hours, from about 2 hours to about 7 hours or from about 4 hours to about 10 hours, from about 4 hours to about 8 hours, or from about 4 hours to about 6 hours. In some embodiments, the blood plasma level of nalbuphine at each time point in the plateau region ranges from about 75% to about 125% of the mean blood plasma level in the plateau region. In some embodiments, the blood plasma level of nalbuphine at each time point in the plateau region ranges from about 80% to about 120% of the mean blood plasma level in the plateau region. In some embodiments, the blood plasma level of nalbuphine at each time point in the plateau region ranges from about 85% to about 115% of the mean blood plasma level in the plateau region. In some embodiments, the blood plasma level of nalbuphine at each time point in the plateau region ranges from about 90% to about 110% of the mean blood plasma level in the plateau region.

In some embodiments, the minimum blood plasma level of nalbuphine observed during the plateau region is not more than about 25% below the mean blood plasma level for all time points in the plateau region. In some embodiments, the minimum blood plasma level of nalbuphine observed during the plateau region is not more than about 20% below the mean blood plasma level in the plateau region. In some embodiments, the minimum blood plasma level of nalbuphine observed during the plateau region is not more than about 15% below the mean blood plasma level in the plateau region. In some embodiments, the minimum blood plasma level of nalbuphine observed during the plateau region ranges from about 75% to about 100% of the mean blood plasma level in the plateau region. In some embodiments, the minimum blood plasma level of nalbuphine observed during the plateau region ranges from about 80% to about 100% of the mean blood plasma level in the plateau region. In some embodiments, the minimum blood plasma level of nalbuphine observed during the plateau region ranges from about 85% to about 100% of the mean blood plasma level in the plateau region. In some embodiments, the minimum blood plasma level of nalbuphine observed during the plateau region ranges from about 80% to about 95% of the mean blood plasma level in the plateau region.

### Co-Therapy

While the compositions can be administered as the sole active pharmaceutical ingredient or sole active anti-pruritus ingredient in the methods described herein, in other embodiments they can also be used in combination with one or more ingredients which are known to be therapeutically effective against pruritus and/or compliment the effect of anti-pruritus ingredient.

For example, in some embodiments, the present methods can employ nalbuphine or a pharmaceutically acceptable salt, solvate or ester thereof in conjunction with one or more anti-pruritic agents. In some embodiments, additional compounds combined with the anti-pruritic agent, e.g., nalbuphine, or a pharmaceutically acceptable salt, solvate or ester thereof, include antihistamines, anti-inflammatory corticosteroids, topical anti-infectives and antifungals, antibacterials, and antivirals, cytotoxic agents, and counter-irritants/analgesics. Other antipruritic agents include anti-depressants, vitamin D, kappa agonists, irritants such as coal tar derivatives and psoralens, 5-HT3 antagonists such as ondansetron, H2 receptor antagonist such as cimetidine, H1 receptor antagonist such as cetirizine, immunomodulators such as tacrolimus, immunosupressants such as cyclosporine A, antagonists, capsaicin, cannabinoids, latex extracts from various Croton species found in the Amazon jungle (e.g., Zangrado), or Nkl antagonists, etc. In some embodiments, nalbuphine or a pharmaceutically acceptable salt, solvate or ester thereof is not administered in combination with a second anti-pruritus agent, e.g., co-formulated or administered separately.

In some embodiments, in nondependent patients nalbuphine does not antagonize an opioid analgesic administered just before, concurrently, or just after nalbuphine. In some other embodiments, patients receiving an opioid analgesic, general anesthetics, phenothiazines, or other tranquilizers, sedatives, hypnotics, or other CNS depressants (including alcohol) concomitantly with nalbuphine exhibit an additive effect. In some other embodiments, when nalbuphine is used in combination with one or more opioid analgesics, general anesthetics, phenothiazines, or other tranquilizers, sedatives, hypnotics, or other CNS depressants (including alcohol), the dosage of one or both agents is reduced.

### Dosing

Formulations employed in the present methods can incorporate nalbuphine in a controlled release formulation such that the formulation provides therapeutically effective blood plasma levels of nalbuphine for the treatment of pruritus. A dosing regimen can be selected for administration of the formulation on a once a day basis, a twice a day basis, thrice a day basis, or four times a day basis. The frequency of dosing can be selected to provide a target plasma concentration of nalbuphine to provide effective relief of the symptoms of pruritus.

In some embodiments, the total daily dose of nalbuphine, either as a single dose or as the sum of two, three or four doses, can range from about 15 mg a day to about 480 mg a day (e.g., about 15 mg a day, about 20 mg a day, about 25 mg a day, about 30 mg a day, about 35 mg a day, about 40 mg a day, about 45 mg a day, about 50 mg a day, about 55 mg a day, about 60 mg a day, about 65 mg a day, about 70 mg a day, about 75 mg a day, about 80 mg a day, about 85 mg a day, about 90 mg a day, about 95 mg a day, about 100 mg a day, about 105 mg a day, about 110 mg a day, about 115 mg a day, about 120 mg a day, about 125 mg a day, about 130 mg a day, about 135 mg a day, about 140 mg a day, about 145 mg a day, about 150 mg a day, about 155 mg a day, about 160 mg a day, about 165 mg a day, about 170 mg a day, about 175 mg a day, about 180 mg a day, about 185 mg a day, about 190 mg a day, about 195 mg a day, about 200 mg a day, about 205 mg a day, about 210 mg a day, about 215 mg a day, about 220 mg a day, about 225 mg a day, about 230 mg a day, about 235 mg a day, about 240 mg a day, about 245 mg a day, about 250 mg a day, about 255 mg a day, about 260 mg a day, about 265 mg a day, about 270 mg a day, about 275 mg a day, about 280 mg a day, about 285 mg a day, about 290 mg a day, about 295 mg a day, about 300 mg a day, about 305 mg a day, about 310 mg a day, about 315 mg a day, about 320 mg a day, about 325 mg a day, about 330 mg a day, about 335 mg a day, about 340 mg a day, about 345 mg a day, about 350 mg a day, about 355 mg a day, about 360 mg a day, about 380 mg, about 400 mg, about 420 mg, about 440 mg, about 460 mg, about 480 mg a day or any other value or range of values therein).

In one embodiment, the total daily dose of nalbuphine, either as a single dose or as the sum of two, three or four doses is 15 mg, 30 mg, 60 mg, 120 mg, 240 mg, 360 mg, or 480 mg. For example, the total daily dose of nalbuphine, either as a single dose or the sum of two doses is 120 mg or 240 mg for the treatment of uremic pruritus. In another example, the total daily dose of nalbuphine, either as a single dose or the sum of two doses is 180 mg or 360 mg for the treatment of a subject who does not have any renal impairment. In certain embodiments, the dose of nalbuphine ranges between one to three 60 mg tablets, twice daily, taken 12 hours apart (one to three tables in the morning and one to three tablets in the evening) for a total daily dose ranging between 120 mg and 360 mg. In some embodiments, the total daily dose of nalbuphine is 120 mg including twice per day and 60 mg/dose. In some other embodiments, the total daily dose of nalbuphine is 240 mg including twice per day and 120 mg/dose. In some other embodiments, the total daily dose of nalbuphine is 360 mg including twice per day and 180 mg/dose.

In some embodiments, the maximum total daily dose of nalbuphine or a pharmaceutically acceptable salt, solvate or ester thereof is about 75 mg to about 180 mg, administered once a day, for example about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, or about 180 mg, administered once a day, including all values or ranges there between.

In some embodiments, the maximum total daily dose of nalbuphine or a pharmaceutically acceptable salt, solvate or ester thereof is about 90 mg to about 360 mg, administered once a day, for example about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about to 60 mg, about 270 mg, about 280 mg, about 290 mg, about 300 mg, about 310 mg, about 320 mg, about 330 mg, about 340 mg, about 350 mg, in about 360 mg, including all values or ranges there between.

In some other embodiments, the maximum total daily dose of nalbuphine or a pharmaceutically acceptable salt, solvate or ester thereof is about 480 mg for a subject either with renal impairment or without renal impairment.

In some embodiments, the dosing regimen is a twice-daily dose of a controlled release formulation, and the amount of nalbuphine in the controlled release formulation is from about 15 mg to about 180 mg (e.g., about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, or any other value or range of values therein). A twice-daily dosage regimen may include a total daily dosage over the two administrations of from about 15 mg a day to about 360 mg a day (e.g., about 15 mg a day, about 20 mg a day, about 25 mg a day, about 30 mg a day, about 35 mg a day, about 40 mg a day, about 45 mg a day, about 50 mg a day, about 55 mg a day, about 60 mg a day, about 65 mg a day, about 70 mg a day, about 75 mg a day, about 80 mg a day, about 85 mg a day, about 90 mg a day, about 95 mg a day, about 100 mg a day, about 105 mg a day, about 110 mg a day, about 115 mg a day, about 120 mg a day, about 125 mg a day, about 130 mg a day, about 135 mg a day, about 140 mg a day, about 145 mg a day, about 150 mg a day, about 155 mg a day, about 160 mg a day, about 165 mg a day, about 170 mg a day, about 175 mg a day, about 180 mg a day, about 185 mg a day, about 190 mg a day, about 195 mg a day, about 200 mg a day, about 205 mg a day, about 210 mg a day, about 215 mg a day, about 220 mg a day, about 225 mg a day, about 230 mg a day, about 235 mg a day, about 240 mg a day, about 245 mg a day, about 250 mg a day, about 255 mg a day, about 260 mg a day, about 265 mg a day, about 270 mg a day, about 275 mg a day, about 280 mg a day, about 285 mg a day, about 290 mg a day, about 295 mg a day, about 300 mg a day, about 305 mg a day, about 310 mg a day, about 315 mg a day, about 320 mg a day, about 325 mg a day, about 330 mg a day, about 335 mg a day, about 340 mg a day, about 345 mg a day, about 350 mg a day, about 355 mg a day, about 360 mg a day, or any other value or range of values therein).

In some embodiments, a twice-daily dosage regimen may include a total daily dosage over the two administrations of from about 60 mg, to about 120 mg, to about 240 mg, to about 360 mg or to about 480 mg.

In some embodiments, a twice-daily dosage regimen may include a total daily dosage over the two administrations of from about 120 mg to 240 mg for subjects with any renal impairment or from about 180 mg to about 360 mg for subject without any renal impairment.

In some embodiments, a twice-daily dosage regimen may include a total daily dosage over the two administrations of from about 60 mg, to about 120 mg, to about 240 mg for subjects without any renal impairment or from about 60 mg to about 120 mg for subjects with any renal impairment.

In some embodiments, a twice-daily dosage regimen may include a total daily dosage over the two administrations of about 60 mg, about 120 mg, or about 180 mg.

In some embodiments, the dosing regimen is a thrice-daily dose of a controlled release formulation, and the amount of nalbuphine in the controlled release formulation is from about 15 mg to about 180 mg (e.g., about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, or any other value or range of values therein). A thrice-daily dosage regimen may include a total daily dosage over the three administrations of from about 15 mg a day to about 360 mg a day (e.g., about 15 mg a day, about 20 mg a day, about 25 mg a day, about 30 mg a day, about 35 mg a day, about 40 mg a day, about 45 mg a day, about 50 mg a day, about 55 mg a day, about 60 mg a day, about 65 mg a day, about 70 mg a day, about 75 mg a day, about 80 mg a day, about 85 mg a day, about 90 mg a day, about 95 mg a day, about 100 mg a day, about 105 mg a day, about 110 mg a day, about 115 mg a day, about 120 mg a day, about 125 mg a day, about 130 mg a day, about 135 mg a day, about 140 mg a day, about 145 mg a day, about 150 mg a day, about 155 mg a day, about 160 mg a day, about 165 mg a day, about 170 mg a day, about 175 mg a day, about 180 mg a day, about 185 mg a day, about 190 mg a day, about 195 mg a day, about 200 mg a day, about 205 mg a day, about 210 mg a day, about 215 mg a day, about 220 mg a day, about 225 mg a day, about 230 mg a day, about 235 mg a day, about 240 mg a day, about 245 mg a day, about 250 mg a day, about 255 mg a day, about 260 mg a day, about 265 mg a day, about 270 mg a day, about 275 mg a day, about 280 mg a day, about 285 mg a day, about 290 mg a day, about 295 mg a day, about 300 mg a day, about 305 mg a day, about 310 mg a day, about 315 mg a day, about 320 mg a day, about 325 mg a day, about 330 mg a day, about 335 mg a day, about 340 mg a day, about 345 mg a day, about 350 mg a day, about 355 mg a day, about 360 mg a day, or any other value or range of values therein).

In some embodiments, the dosing regimen is a once-daily dose, and the amount of nalbuphine in the formulation is from about 15 mg to about 180 mg (e.g., about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, or any other value or range of values therein).

In some embodiments, irrespective of the dosing regimen, the controlled release formulation comprises nalbuphine or a pharmaceutically acceptable salt, solvate or ester thereof of about 15 mg, about 30 mg, about 60 mg, about 90 mg, about 120 mg, or about 180 mg.

In some embodiments, the daily dose of nalbuphine can be selected as described above, in either a once-daily dose, a twice daily dose, or a thrice-daily dose, and then titrated upward until the patient experiences satisfactory relief from the pruritic condition. Titrating the dose can include administering a baseline daily dose, in either a once-daily dose, a twice daily dose, or a thrice daily dose, then after a period of observation at the baseline daily dose value to determine the efficacy of the baseline first daily dose and/or side effect severity, increasing the first daily dose if the subject does not experience adequate symptom relief. The period of observation at the baseline daily dose before increasing the daily dose can be from about 1 day to about 21 days (e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days). The daily dose can be titrated in increments ranging from about 5 mg to about 180 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, or any other value or range of values therein). The daily dose can be titrated in one or more steps. The daily dosage can be titrated by increasing a single daily dosage, or each dose of a twice-daily dosing regimen. The amount a dosage is stepped, where there are multiple titration steps, can be the same, or can be different. In some embodiments, a dosage in, e.g., a twice-daily or thrice-daily dosing regimen can be titrated downward, while the corresponding second dose (e.g., in a twice-daily regimen) or the corresponding second and third doses (e.g., in a twice-daily regimen) can independently be held constant or increased, to reduce the total number of doses per day while retaining therapeutic efficacy.

In some embodiments, nalbuphine or a pharmaceutically acceptable salt, solvate or ester is administered once a day at about 15 mg to about 30 mg, then twice a day at about 30 mg per dose for about 2 to 3 days, and then increased to twice a day at about 60 mg or 120 mg per dose, e.g., for subjects with uremic pruritus or renal impairment. In some other embodiments, nalbuphine or a pharmaceutically acceptable salt, solvate or ester is administered once a day at about 15-30 mg, then twice a day at about 30 mg per dose for about 2 to 3 days, and then increased to twice a day at about 90 mg or 180 mg per dose, e.g., for subjects without any renal impairment.

In some other embodiments, nalbuphine or a pharmaceutically acceptable salt, solvate or ester may be initiated with the 30 mg tablet twice daily (at 12-hour intervals). In certain embodiments, doses can be adjusted in 30 mg increments every 3 to 4 days. Subjects can self-titrate to effect over 21 days to a dose that provides adequate relief from itch and minimizes adverse reactions. In certain other embodiments, subjects can be provided initially with 30 mg tablets sufficient to self-titrate to effect up to 180 mg twice a day over 3 weeks of therapy as clinical benefits should generally be identified within 3-weeks after starting nalbuphine, nalbuphine HCl. In certain other embodiments, a VAS score review is recommended at three weeks to evaluate the improvement (reduction) in itch after three weeks. In certain embodiments, subjects can be maintained at the highest dose reached after 3 weeks and reported by the subjects to be beneficial. Physicians may consider re-assessing treatment and dose level relative to the benefits of nalbuphine.

During chronic therapy, physicians can also consider re-assessing treatment and dose level relative to the benefits of nalbuphine. The doses of nalbuphine can range between one to three 60 mg tablets, twice daily, taken 12 hours apart (one to three tablets in the morning and one tablet to three tablets in the evening). Nalbuphine can be discontinued if patients no longer draw benefits from the treatment.

When nalbuphine HCl solution is administered with food, AUC is relatively unchanged whereas Cmax values are about 1.5 fold higher in the fed than the fasted state. Exposure (AUC) following tablets was comparable to that from the solution with a relative bioavailability of 94% under fasted state. On the other hand, with sustained release nalbuphine formulations, Cmax was blunted (˜50%) and Tmax prolonged both in the fed and fasted state relative to the nalbuphine HCl solution. In one embodiment, following oral administration of an aqueous solution, nalbuphine is readily absorbed with a median Tmax of 0.5, 1, 2, 3, 4, 5, 6, 7, or 8 hrs and a mean plasma half-life (T_{1/2}) ranging between about 4 to about 20 hrs. In another embodiment, following oral administration of an aqueous solution, nalbuphine is readily absorbed with a median Tmax of 0.5-1 hr and a mean plasma half-life (T_{1/2}) ranging between 6.87 and 6.99 hr across studies under fasted conditions.

In some embodiments, the dosing frequency and dose amount per administration are selected to provide therapeutically effective blood plasma levels of nalbuphine for the treatment of pruritus when administered on a once-a-day or twice-a-day basis. For example, in certain embodiments, the controlled release formulation, provides a mean Cₘₐₓ of from about 1.0 to about 120 ng/mL, for example, a mean Cₘₐₓ of about 1.0, about 1.1, about 1.2, about 1.3, about 1.4, about 1.5, about 1.6, about 1.7, about 1.8, about 1.9, about 2.0, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0, about 5.2, about 5.4, about 5.6, about 5.8, about 6.0, about 6.2, about 6.4, about 6.6, about 6.8, about 7.0, about 7.2, about 7.4, about 7.6, about 7.8, about 8.0, about 8.2, about 8.4, about 8.6, about 8.8, about 9.0, about 9.2, about 9.4, about 9.6, about 9.8, about 10, about 11, about 12, about 13, about 14, about 15, about 16, about 17, about 18, about 19, about 20, about 21, about 22, about 23, about 24, about 25, about 26, about 27, about 28, about 29, about 30, 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, 81, about 82, about 83, about 84, about 85, about 86, about 87, about 88, about 89, about 90, about 91, about 92, about 93, about 94, about 95, about 96, about 97, about 98, about 99, about 100, about 101, about 102, about 1 to 3, about 104, about 105, about 16, about 107, about 108, about 109, about 110, about 111, about 112, about 113, about 114, about 115, about 116, about 117, about 118, about 119, about 120 ng/mL, or any other value or range of values therein.

In one embodiment, the controlled release formulation provides a mean Cₘₐₓ of from about 1.9 ng/mL to about 102 ng/mL. In another embodiment, the controlled release formulation provides a mean Cₘₐₓ of from about 30 ng/mL to about 60 ng/mL. In yet another embodiment, the controlled release formulation provides a mean Cₘₐₓ of from about 2 ng/mL to about 15 ng/mL. In yet another embodiment, the controlled release formulation provides a mean Cₘₐₓ of from about 5 ng/mL to about 10 ng/mL. In still other embodiments, the controlled release formulation provides a mean Cₘₐₓ of from about 10 ng/mL to about 20 ng/mL. In still other embodiments, the controlled release formulation provides a mean Cₘₐₓ of from about 20 ng/mL to about 30 ng/mL. In still other embodiments, the controlled release formulation provides a mean Cₘₐₓ of from about 30 ng/mL to about 50 ng/mL. In still other embodiments, the controlled release formulation provides a mean Cₘₐₓ of from about 50 ng/mL to about 60 ng/mL.

In one embodiment, the controlled release formulation provides a mean Cₘₐₓ of from about 5 ng/mL to about 85 ng/mL, e.g., for subjects with uremic pruritus or renal impairment or from about 5 ng/mL to about 45 ng/mL, e.g., for subjects without any renal impairment. For example, the controlled release formulation provides a mean Cₘₐₓ of from about 24 ng/mL to about 71 ng/mL, e.g., for subjects with uremic pruritus or renal impairment or from about 13 ng/mL to about 28 ng/mL, e.g., for subjects without any renal impairment. In another embodiment, the controlled release formulation provides a mean Cₘₐₓ of from about 24 ng/mL to about 83 ng/mL, e.g., for subjects with uremic pruritus or renal impairment or from about 13 ng/mL to about 44 ng/mL, e.g., for subjects without any renal impairment. In yet another embodiment, the controlled release formulation provides a mean Cₘₐₓ of from about 18 ng/mL, 19 ng/mL, 20 ng/mL, 21 ng/mL, 22 ng/mL, 23 ng/mL, 24 ng/mL, 25 ng/mL, 26 ng/mL, 27 ng/mL, 28 ng/mL, or 30 ng/mL to about 75 ng/mL, 76 ng/mL, 77 ng/mL, 78 ng/mL, 79 ng/mL, 80 ng/mL, 81 ng/mL, 82 ng/mL, 83 ng/mL, 84 ng/mL, 85 ng/mL, 86 ng/mL, 87 ng/mL, 88 ng/mL, 89 ng/mL, or 90 ng/mL e.g., for subjects with uremic pruritus or renal impairment or from about 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, or 18 ng/mL to about 36 ng/mL, 37 ng/mL, 38 ng/mL, 39 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, or 50 ng/mL e.g., for subjects without any renal impairment.

In other embodiments, the present formulations provide a mean Cmax from about 0.088 (ng/mL)/mg to about 0.245 (ng/mL)/mg (e.g., about 0.08 (ng/mL)/mg, about 0.09 (ng/mL)/mg, about 0.1 (ng/mL)/mg, about 0.11 (ng/mL)/mg, about 0.12 (ng/mL)/mg, about 0.13 (ng/mL)/mg, about 0.14 (ng/mL)/mg, about 0.15 (ng/mL)/mg, about 0.16 (ng/mL)/mg, about 0.17 (ng/mL)/mg, about 0.18 (ng/mL)/mg, about 0.19 (ng/mL)/mg, about 0.20 (ng/mL)/mg, about 0.21 (ng/mL)/mg, about 0.22 (ng/mL)/mg, about 0.23 (ng/mL)/mg, about 0.24 (ng/mL)/mg, or any other value or range of values therein). In some embodiments, the present formulations provide a mean Cmax from about 0.15 (ng/mL)/mg to about 0.35 (ng/mL)/mg. For example, according to the present invention the sustained release formulation can have a mean Cmax from about 0.2 (ng/mL)/mg to about 0.3 (ng/mL)/mg.

In one embodiment, the present formulations provide a mean Cmax from about 0.2 (ng/mL)/mg to about 0.6 (ng/mL)/mg, e.g., for subjects with uremic pruritus or renal impairment or from about 0.15 (ng/mL)/mg to about 0.25 (ng/mL)/mg, e.g., for subjects without any renal impairment. For example, the present formulations provide a mean Cmax from about 0.4 (ng/mL)/mg to about 0.6 (ng/mL)/mg, e.g., for subjects with uremic pruritus or renal impairment or from about 0.2 (ng/mL)/mg to about 0.3 (ng/mL)/mg, e.g., for subjects without any renal impairment.

In some embodiments, the dosing frequency and dose amount per administration are selected to provide a mean Cₘᵢₙ of from about 1 ng/mL to about 20 ng/mL (e.g., about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, about 20 ng/mL, or any other value or range of values therein). In certain embodiments, the dosing frequency and dose amount per administration are selected to provide a mean Cₘᵢₙ of from about 2 ng/mL to about 15 ng/mL. In other embodiments, the dosing frequency and dose amount per administration are selected to provide a mean Cₘᵢₙ of from about 5 ng/mL to about 10 ng/mL, 10 ng/mL to about 20 ng/mL, 20 ng/mL to about 30 ng/mL, 30 ng/mL to about 40 ng/mL, 40 ng/mL to about 50 ng/mL or 50 ng/mL to about 60 ng/mL.

In one embodiment, the dosing frequency and dose amount per administration are selected to provide a mean Cₘᵢₙ of from about 2 ng/mL to about 45 ng/mL, e.g., for subjects with uremic pruritus or renal impairment and optionally a pre AM dose or from about 0.5 ng/mL to about 25 ng/mL for subjects without any renal impairment and optionally a pre AM dose. In another embodiment, the dosing frequency and dose amount per administration are selected to provide a mean Cₘᵢₙ of from about 5 ng/mL to about 60 ng/mL, e.g., for subjects with uremic pruritus or renal impairment and optionally a pre PM dose or from about 2 ng/mL to about 20 ng/mL for subjects without any renal impairment and optionally a pre PM dose.

In some embodiments, the controlled release formulation, provides a median Tₘₐₓ of from about 1 to about 10, for example, a median Tₘₐₓ of about 1, about 2, about 3, about 4, about 5, about 6, about 7, about 8, about 9, about 10, or any other value or range of values therein.

In certain embodiments, the controlled release formulation provides a median Tₘₐₓ of from about 5 to about 6, e.g., for subjects with uremic pruritus or renal impairment or from about 3 to about 5, e.g., for subjects without any renal impairment.

In some embodiments, the controlled release formulation, provides a mean plasma half-life (T_{1/2}) of from about 1 hr to about 50 hr, for example, a mean plasma half-life (T_{1/2}) of about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 50 hr or any other value or range of values therein.

In certain embodiments, the controlled release formulation provides a mean plasma half-life (T_{1/2}) of from about 10 to about 20 hr, e.g., for subjects with uremic pruritus or renal impairment or from about 6 hr to about 9 hr, e.g., for subjects without any renal impairment.

In certain embodiments, the dosing frequency and dose amount of the controlled release formulation provides a nalbuphine AUC_{(0-∞)} of from about 30 ng·hr/mL to about 950 ng·hr/mL, for example about 30, about 31, about 32, about 33, about 34, about 35, about 36, about 37, about 38, about 39, about 40, about 45, about 50, about 55, about 60, about 65, 70, 75 about 80, 85, about 90, about 95, about 100, about 105, about 110, about 115, about 120, about 125, about 130, about 135, about 140, about 145, about 150, about 155, about 160, 165, about 170, about 175, about 180, about 185, about 190, about 195, about 200, about 205, about 210, about 215, about 220, about 225, about to 30, about 235, about 240, about 245, about 250 about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 35, about 390, about 395, about 400, about 405, about 410, about 415, about 420, about 425, about 430, about 435, about 440, about 445, about 450, about 455, about 460, about 465, about 470, about 475, about 480, about 485, about 490, about 495, about 500, about 505, about 510, about 515, about 520, about 525, about 530, about 535, about 540, about 545, about 550, about 555, about 560, about 565, about 570, about 575, about 580, about 585, about 588, about 590, about 595, about 600, about 605, about 610, about 615, about 620, about 625, about 630, about 635, about 640 about 645, about 650, about 655, about 670, about 675, about 680, about 685, about 690, about 695, about 700, about 705, about 710, about 715, about 720, about 725, about 730, about 735, about 740, about 745, about 750, about 755, about 760, about 765, about 770, about 775, about 780, about 785, about 790, about 795, about 800, about 805, about 810, about 815, about 820, about 825, about 830, about 835, about 840, about 845, about 850, about 855, about 860, but 865, about 870, about 875, about 880, about 885, about 890, about 895, about 900, about 905, about 910, about 915, about 920, about 925, about 930, about 935, about 940, about 945, about 950 ng·hr/mL, or any other value or range of values therein.

In one embodiment, the dosing frequency and dose amount of the controlled release formulation provides a nalbuphine AUC_{(0-∞)} of from about 37 ng·hr/mL to about 910 ng·hr/mL. In another embodiment, the controlled release formulation provides a nalbuphine AUC_{(0-∞)} of from about 200 ng·hr/mL to about 500 ng·hr/mL. In another embodiment, the controlled release formulation provides a nalbuphine AUC_{(0-∞)} of from about 70 ng·hr/mL to about 210 ng·hr/mL. In yet another embodiment, the controlled release formulation provides a nalbuphine AUC_{(0-∞)} of from about 50 ng·hr/mL to about 800 ng·hr/mL. In still another embodiment, the controlled release formulation provides a nalbuphine AUC_{(0-∞)} of from about 60 ng·hr/mL to about 720 ng·hr/mL. In still another embodiment, the controlled release formulation provides a nalbuphine AUC_{(0-∞)} of from about 60 ng·hr/mL to about 80 ng·hr/mL.

In some embodiments, the dosing frequency and dose amount of the controlled release formulation provides a nalbuphine AUC_{(0-∞)} of from about 142 ng·hr/mL to about 2640 ng·hr/mL, e.g., for subjects with uremic pruritus or renal impairment or from about 49 ng·hr/mL to about 600 ng·hr/mL, e.g., for subjects without any renal impairment. In certain embodiments, the dosing frequency and dose amount of the controlled release formulation provides a nalbuphine AUC_{(0-∞)} of from about 500 ng·hr/mL to about 600 ng·hr/mL, e.g., for subjects without any renal impairment. In certain embodiments, the dosing frequency and dose amount of the controlled release formulation provides a nalbuphine AUC_{(0-∞)} of about 588 ng·hr/mL, e.g., for subjects without any renal impairment.

In other embodiments, the present formulations provide a nalbuphine AUC_{(0-inf)} from about 1.392 (ng*hr/ml)/mg to about 3.43 (ng*hr/ml)/mg (e.g., about 1.4 (ng*hr/ml), about 1.5 (ng*hr/ml), about 1.6 (ng*hr/ml), about 1.7 (ng*hr/ml), about 1.8 (ng*hr/ml), about 1.9 (ng*hr/ml), about 2.0 (ng*hr/ml), about 2.1 (ng*hr/ml), about 2.2 (ng*hr/ml), about 2.3 (ng*hr/ml), about 2.4 (ng*hr/ml), about 2.5 (ng*hr/ml), about 2.6 (ng*hr/ml), about 2.7 (ng*hr/ml), about 2.8 (ng*hr/ml), about 2.9 (ng*hr/ml), about 3.0 (ng*hr/ml), about 3.1 (ng*hr/ml), about 3.2 (ng*hr/ml), about 3.3 (ng*hr/ml), about 3.4 (ng*hr/ml), or any other value or range of values therein.

In some embodiments, the present formulations provide a nalbuphine AUC_{(0-inf)} from about 20 (ng*hr/ml)/mg to about 500 (ng*hr/ml)/mg. In some other embodiments, the present formulations provide a nalbuphine AUC_{(0-inf)} from about 30 (ng*hr/ml)/mg to about 450 (ng*hr/ml)/mg. In yet some other embodiments, the present formulations provide a nalbuphine AUC_{(0-inf)} from about 30 (ng*hr/ml)/mg to about 150 (ng*hr/ml)/mg.

In some other embodiments, the dosing frequency and dose amount of the controlled release formulation provides a nalbuphine AUC_{(0-inf)} of about 500 hr·ng/mL, about 520 hr·ng/mL, about 550 hr·ng/mL, about 580 hr·ng/mL, or about 600 hr·ng/mL at 180 mg BID, e.g., for subjects without any renal impairment. In some other embodiments, the dosing frequency and dose amount of the controlled release formulation provides a nalbuphine AUC_{(0-inf)} ranging from about 500 hr·ng/mL to about 600 hr·ng/mL at 180 mg BID, e.g., for subjects without any renal impairment.

In yet some other embodiments, the dosing frequency and dose amount of the controlled release formulation provides a mean Cₘₐₓ of about 13 ng/mL and AUC_{(0-inf)} of about 20 hr·ng/mL at 30 mg BID, e.g., for subjects with uremic pruritus or renal impairment. In yet some other embodiments, the dosing frequency and dose amount of the controlled release formulation provides a mean Cₘₐₓ of about 25 ng/mL and AUC_{(0-inf)} of about 1000 hr·ng/mL at 60 mg BID, e.g., for subjects with uremic pruritus or renal impairment. In yet some other embodiments, the dosing frequency and dose amount of the controlled release formulation provides a mean Cₘₐₓ of about 70 ng/mL and AUC_{(0-inf)} of about 2000 hr·ng/mL at 120 mg BID, e.g., for subjects with uremic pruritus or renal impairment. In yet some other embodiments, the dosing frequency and dose amount of the controlled release formulation provides a mean Cₘₐₓ of about 83 ng/mL and AUC_{(0-inf)} of about 2600 hr·ng/mL at 180 mg BID, e.g., for subjects with uremic pruritus or renal impairment. In yet some other embodiments, the dosing frequency and dose amount of the controlled release formulation provides a mean Cₘₐₓ of about 61 ng/mL and AUC_{(0-inf)} of about 1600 hr·ng/mL at 240 mg BID, e.g., for subjects with uremic pruritus or renal impairment.

In some embodiments, the dosing frequency and dose amount of the controlled release formulation provides a mean Cₘₐₓ of about 6.5 ng/mL and AUC_{(0-inf)} of about 20 hr·ng/mL at 30 mg BID, e.g., for subjects without any renal impairment. In some other embodiments, the dosing frequency and dose amount of the controlled release formulation provides a mean Cₘₐₓ of about 13 ng/mL and AUC_{(0-inf)} of about 300 hr·ng/mL at 60 mg BID, e.g., for subjects without any renal impairment. In some other embodiments, the dosing frequency and dose amount of the controlled release formulation provides a mean Cₘₐₓ of about 28 ng/mL and AUC_{(0-inf)} of about 400 hr·ng/mL at 120 mg BID, e.g., for subjects without any renal impairment. In some embodiments, the dosing frequency and dose amount of the controlled release formulation provides a mean Cₘₐₓ of about 44 ng/mL and AUC_{(0-inf)} of about 590 hr·ng/mL at 180 mg BID, e.g., for subjects without any renal impairment.

In certain embodiments, the dosing frequency and dose amount of the controlled release formulation provides a nalbuphine AUCₜₐᵤ of from about 30 ng·hr/mL to about 950 ng·hr/mL, for example about 30, about 31, about 32, about 33, about 34, about 35, about 36, about 37, about 38, about 39, about 40, about 45, about 50, about 55, about 60, about 65, 70, 75 about 80, 85, about 90, about 95, about 100, about 105, about 110, about 115, about 120, about 125, about 130, about 135, about 140, about 145, about 150, about 155, about 160, 165, about 170, about 175, about 180, about 185, about 190, about 195, about 200, about 205, about 210, about 215, about 220, about 225, about to 30, about 235, about 240, about 245, about 250 about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 35, about 390, about 395, about 400, about 405, about 410, about 415, about 420, about 425, about 430, about 435, about 440, about 445, about 450, about 455, about 460, about 465, about 470, about 475, about 480, about 485, about 490, about 495, about 500, about 505, about 510, about 515, about 520, about 525, about 530, about 535, about 540, about 545, about 550, about 555, about 560, about 565, about 570, about 575, about 580, about 585, about 588. about 590, about 595, about 600, about 605, about 610, about 615, about 620, about 625, about 630, about 635, about 640 about 645, about 650, about 655, about 670, about 675, about 680, about 685, about 690, about 695, about 700, about 705, about 710, about 715, about 720, about 725, about 730, about 735, about 740, about 745, about 750, about 755, about 760, about 765, about 770, about 775, about 780, about 785, about 790, about 795, about 800, about 805, about 810, about 815, about 820, about 825, about 830, about 835, about 840, about 845, about 850, about 855, about 860, but 865, about 870, about 875, about 880, about 885, about 890, about 895, about 900, about 905, about 910, about 915, about 920, about 925, about 930, about 935, about 940, about 945, about 950 ng·hr/mL, or any other value or range of values therein.

In certain embodiments, the dosing frequency and dose amount of the controlled release formulation provides a nalbuphine AUCₜₐᵤ of from about 40 ng·hr/mL to about 800 ng·hr/mL (about 50 ng·hr/mL, about 60 ng·hr/mL, about 70 ng·hr/mL, about 80 ng·hr/mL, about 90 ng·hr/mL, about 100 ng·hr/mL, about 120 ng·hr/mL, about 140 ng·hr/mL, about 160 ng·hr/mL, about 180 ng·hr/mL, about 200 ng·hr/mL, about 220 ng·hr/mL, about 300 ng·hr/mL, about 400 ng·hr/mL, about 500 ng·hr/mL, about 600 ng·hr/mL, about 620 ng·hr/mL, about 700 ng·hr/mL, about 750 ng·hr/mL, about 770 ng·hr/mL, or any other value or range of values therein), e.g., for subjects with uremic pruritus or renal impairment or from about 30 ng·hr/mL to about 360 ng·hr/mL (about 40 ng·hr/mL, about 50 ng·hr/mL, about 60 ng·hr/mL, about 70 ng·hr/mL, about 80 ng·hr/mL, about 90 ng·hr/mL, about 100 ng·hr/mL, about 120 ng·hr/mL, about 140 ng·hr/mL, about 160 ng·hr/mL, about 180 ng·hr/mL, about 200 ng·hr/mL, about 220 ng·hr/mL, about 240 ng·hr/mL, about 260 ng·hr/mL, about 280 ng·hr/mL, about 300 ng·hr/mL, about 320 ng·hr/mL, about 350 ng·hr/mL or any other value or range of values therein), e.g., for subjects without any renal impairment.

In certain embodiments, the dosing frequency and dose amount of the controlled release formulation provides a nalbuphine AUCₜₐᵤ of from about 221 ng·hr/mL to about 621 ng·hr/mL, e.g., for subjects with uremic pruritus or renal impairment. In certain embodiments, the dosing frequency and dose amount of the controlled release formulation provides a nalbuphine AUCₜₐᵤ of from about 51 ng·hr/mL to about 351 ng·hr/mL, or from about 106 ng·hr/mL to about 351 ng·hr/mL, e.g., for subjects without any renal impairment.

In certain embodiments, the dosing frequency and dose amount of the controlled release formulation provides a mean Cₘₐₓ of about 13 ng/mL and AUCₜₐᵤ of about 118 hr·ng/mL at 30 mg BID, e.g., for subjects with uremic pruritus or renal impairment. In certain embodiments, the dosing frequency and dose amount of the controlled release formulation provides a mean Cₘₐₓ of about 25 ng/mL and AUCₜₐᵤ of about 221 hr·ng/mL at 60 mg BID, e.g., for subjects with uremic pruritus or renal impairment. In certain embodiments, the dosing frequency and dose amount of the controlled release formulation provides a mean Cₘₐₓ of about 70 ng/mL and AUCₜₐᵤ of about 621 hr·ng/mL at 120 mg BID, e.g., for subjects with uremic pruritus or renal impairment. In certain embodiments, the dosing frequency and dose amount of the controlled release formulation provides a mean Cₘₐₓ of about 82 ng/mL and AUCₜₐᵤ of about 760 hr·ng/mL at 180 mg BID, e.g., for subjects with uremic pruritus or renal impairment.

In certain embodiments, the dosing frequency and dose amount of the controlled release formulation provides a mean Cₘₐₓ of about 6 ng/mL and AUCₜₐᵤ of about 50 hr·ng/mL at 30 mg BID, e.g., for subjects without any renal impairment. In certain embodiments, the dosing frequency and dose amount of the controlled release formulation provides a mean Cₘₐₓ of about 13 ng/mL and AUCₜₐᵤ of about 106 hr·ng/mL at 60 mg BID, e.g., for subjects without any renal impairment. In certain embodiments, the dosing frequency and dose amount of the controlled release formulation provides a mean Cₘₐₓ of about 28 ng/mL and AUCₜₐᵤ of about 240 hr·ng/mL at 120 mg BID, e.g., for subjects without any renal impairment. In certain embodiments, the dosing frequency and dose amount of the controlled release formulation provides a mean Cₘₐₓ of about 44 ng/mL and AUCₜₐᵤ of about 351 hr·ng/mL at 180 mg BID, e.g., for subjects without any renal impairment.

In certain embodiments, the present methods can further comprise administering a rescue dose comprising nalbuphine to provide breakthrough relief of pruritus. The rescue dose can comprise nalbuphine in an amount of from about 1 mg to about 60 mg (e.g., about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, or any other value or range of values therein). In some embodiments, the rescue dose comprises from about 3 mg to about 30 mg of nalbuphine. In other embodiments, the rescue dose comprises from about 3 mg to about 45 mg of nalbuphine. The rescue dose can be administered parenterally, orally in an immediate release formulation, or as a buccal, sublingual, intranasal, or rectal dosage form. In some embodiments, the rescue dose is a tablet, capsule, a solution, a lozenge, or a suppository. In other embodiments, the rescue dose can be administered via a bilayer tablet which includes a recue nalbuphine dose in an immediate release layer, and the tablet further comprises a nalbuphine dose in an extended release layer.

In some embodiments, the present methods employ a twice daily dosage regimen, and the first daily dose is less than the second daily dose. For example, in some embodiments, the second daily dose is from about 5 mg to about 180 mg greater than the first daily dose (e.g., about 5 mg greater, about 10 mg greater, about 15 mg greater, about 20 mg greater, about 25 mg greater, about 30 mg greater, about 35 mg greater, about 40 mg greater, about 45 mg greater, about 50 mg greater, about 55 mg greater, about 60 mg greater, about 65 mg greater, about 70 mg greater, about 75 mg greater, about 80 mg greater, about 85 mg greater, about 90 mg greater, about 95 mg greater, about 100 mg greater, about 105 mg greater, about 110 mg greater, about 115 mg greater, about 120 mg greater, about 125 mg greater, about 130 mg greater, about 135 mg greater, about 140 mg greater, about 145 mg greater, about 150 mg greater, about 155 mg greater, about 160 mg greater, about 165 mg greater, about 170 mg greater, about 175 mg greater, about 180 mg greater, or any other value or range of values therein).

In some embodiments, the second daily dose of nalbuphine can be selected as described above, and then titrated upward until the patient experiences satisfactory relief from the pruritic condition. Titrating the dose can include administering a baseline second daily dose, then after a period of observation at the baseline second daily dose value to determine the efficacy and/or side effect severity, of the baseline second daily dose, increasing the second daily dose if the subject does not experience adequate symptom relief. The period of observation at the baseline second daily dose before increasing the second daily dose can be from about 1 day to about 21 days (e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days). The second daily dose can be titrated in increments ranging from about 5 mg to about 180 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, or any other value or range of values therein). The second daily dose can be titrated in one or more steps. The amount a dosage is stepped, where there are multiple titration steps, can be the same, or can be different.

In some embodiments, the present methods employ a twice daily dosage regimen, and the first daily dose is greater than the second daily dose. For example, in some embodiments, the first daily dose is from about 5 mg to about 180 mg greater than the second daily dose (e.g., about 5 mg greater, about 10 mg greater, about 15 mg greater, about 20 mg greater, about 25 mg greater, about 30 mg greater, about 35 mg greater, about 40 mg greater, about 45 mg greater, about 50 mg greater, about 55 mg greater, about 60 mg greater, about 65 mg greater, about 70 mg greater, about 75 mg greater, about 80 mg greater, about 85 mg greater, about 90 mg greater, about 95 mg greater, about 100 mg greater, about 105 mg greater, about 110 mg greater, about 115 mg greater, about 120 mg greater, about 125 mg greater, about 130 mg greater, about 135 mg greater, about 140 mg greater, about 145 mg greater, about 150 mg greater, about 155 mg greater, about 160 mg greater, about 165 mg greater, about 170 mg greater, about 175 mg greater, about 180 mg greater, or any other value or range of values therein).

In some embodiments, the first daily dose of nalbuphine can be selected as described above, and then titrated upward until the patient experiences satisfactory relief from the pruritic condition. Titrating the dose can include administering a baseline first daily dose, then after a period of observation at the baseline first daily dose value to determine the efficacy of the baseline first daily dose and/or side effect severity, increasing the first daily dose if the subject does not experience adequate symptom relief. The period of observation at the baseline first daily dose before increasing the first daily dose can be from about 1 day to about 21 days (e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days). The first daily dose may be from about 5 mg to about 180 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, or any other value or range of values therein).

The titration dose can be in increments ranging from about 5 mg to about 180 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, or any other value or range of values therein). The first daily dose can be titrated in one or more steps. The amount a dosage is stepped, where there are multiple titration steps, can be the same, or can be different.

Accordingly, the total daily dose in a once daily dosing regimen with a titration dose, including baseline dose and one or more titration doses, may be from about 5 mg to about 180 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, or any other value or range of values therein).

In one embodiment, the initial dose is about 15 mg to about 30 mg once a day for a day or two, then about 30 mg twice a day for 2 to 3 days and then increase to about 60 mg or 120 mg twice a day, e.g., for subjects with uremic pruritus or renal impairment or about 90 mg or 180 mg twice a day, e.g., for subjects without renal impairment.

In one embodiment, the present methods include treating pruritus in a human patient, comprising (a) orally administering to a human patient suffering from pruritus an initial dosing regimen of nalbuphine in a controlled release formulation containing about 15 mg, 30 mg or 60 mg nalbuphine on a once a day or twice-a-day basis; (b) determining the effectiveness of the dosing regimen of nalbuphine in treating the patient's pruritus after at least about 1, 2 or 3 days of treatment; and (c) adjusting the dose and/or dosing interval of the oral controlled release nalbuphine formulation based on the information obtained in step (b) in order to provide improved efficacy of treatment of said patient's pruritus.

In other embodiments, the initial dosing is about 15 mg, 30 mg, or 60 mg once a day or twice a day and an effective dosing is obtained via titration, e.g., based on a pre-determined schedule, e.g., about 15 mg, 30 mg or 60 mg increment after about 2 or 3 days or based on a schedule designed by the physician treating the patient.

In some other embodiments, the maximum daily dose is about 75 mg, 150 mg, 180 mg, or 240 mg once a day, or 240 mg, 360 mg, 480 mg, 600 mg, or 720 mg twice a day. In one embodiment, the maximum daily dose is about 240 mg once a day or 480 mg twice a day, e.g., for subjects with or without renal impairment.

In some embodiments, the effectiveness of a dosage regimen can be determined by evaluation via a Pruritis Visual Analog Scale (VAS) test. In one embodiment, the effectiveness of a dosage regimen can be determined by evaluation via a Numerical Rating Scale (NRS). In yet some other embodiments, the effectiveness of a dosage regimen can be determined by evaluation via a Numerical Rating Scale (NRS) in association with Medical Outcomes Study (MOS), Itch MOS Sleep scale, Hospital Anxiety and Depression Scale (HADS), Patient Assessed Disease Severity Scale, or Skindex-10 or a combination thereof. In still another embodiment, the effectiveness of a dosage regimen can be determined by evaluation via a NRS independently or in association with Patient Global Assessment (PGA) via ItchApp, vPGA, Dermatology Life Quality Index (DLQI), Patient Benefit Index (PBI), MOS Sleep Scale, or HADS or a combination thereof. In still yet another embodiment, the effectiveness of a dosage regimen can be determined by evaluation via a NRS independently or in association with Prurigo Activity Score (PAS), Nocturnal scratching using actigraphy, nerve fiber density and MOR/KOR density.

In other embodiments, determining the effectiveness of the dose of nalbuphine can include evaluation of the patient's pruritus symptoms using an instrument selected from the group consisting of Pruritis Visual Analog Scale (VAS) test, Brief Itching Inventory, Skindex-10, Itch MOS of sleep, Beck Depression Index, and Patient Categorization of Pruritus Disease Severity.

In some embodiments, a subject, e.g., a human or animal patient can be treated initially with a twice-daily dosing regimen, and then after titrating either the first daily dose, the second daily dose, or both to arrive at a total daily dose effective to treat the patient's pruritus symptoms, the dosage regimen can be changed to a once-daily dosage. Each dose of the twice daily dosage can then be further titrated as described hereinabove to provide a therapeutically effective once daily dosage of nalbuphine. Thus, in some embodiments, the dosage regimen is a twice-daily dose, and each of the first and second doses can be titrated to achieve maximum therapeutic effect and/or minimal side effect severity. Accordingly, in some embodiments each of the first and second doses in a twice-daily dosage regimen may be from about 5 mg to about 240 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 190 mg, about 200 mg, about 210 mg, about 220 mg, about 230 mg, about 240 mg or any other value or range of values therein).

In some embodiments, each of the first and second daily doses in a twice daily dosage regimen may be independently titrated in an amount of from about 5 mg to about 240 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 190 mg, about 200 mg, about 210 mg, about 220 mg, about 230 mg, about 240 mg or any other value or range of values therein). The first and second doses in a twice-daily dosage regimen may be independently titrated in one or more steps. The amount a dosage is stepped, where there are multiple titration steps, can be the same, or can be different.

Accordingly, the total daily dose in a twice daily dosing regimen, including baseline dose for each administration and one or more titration doses for each administration independently, may be from about 5 mg a day to about 480 mg a day (e.g., about 15 mg a day, about 20 mg a day, about 25 mg a day, about 30 mg a day, about 35 mg a day, about 40 mg a day, about 45 mg a day, about 50 mg a day, about 55 mg a day, about 60 mg a day, about 65 mg a day, about 70 mg a day, about 75 mg a day, about 80 mg a day, about 85 mg a day, about 90 mg a day, about 95 mg a day, about 100 mg a day, about 105 mg a day, about 110 mg a day, about 115 mg a day, about 120 mg a day, about 125 mg a day, about 130 mg a day, about 135 mg a day, about 140 mg a day, about 145 mg a day, about 150 mg a day, about 155 mg a day, about 160 mg a day, about 165 mg a day, about 170 mg a day, about 175 mg a day, about 180 mg a day, about 185 mg a day, about 190 mg a day, about 195 mg a day, about 200 mg a day, about 205 mg a day, about 210 mg a day, about 215 mg a day, about 220 mg a day, about 225 mg a day, about 230 mg a day, about 235 mg a day, about 240 mg a day, about 245 mg a day, about 250 mg a day, about 255 mg a day, about 260 mg a day, about 265 mg a day, about 270 mg a day, about 275 mg a day, about 280 mg a day, about 285 mg a day, about 290 mg a day, about 295 mg a day, about 300 mg a day, about 305 mg a day, about 310 mg a day, about 315 mg a day, about 320 mg a day, about 325 mg a day, about 330 mg a day, about 335 mg a day, about 340 mg a day, about 345 mg a day, about 350 mg a day, about 355 mg a day, about 360 mg a day, about 390 mg, about 420 mg, about 450 mg, about 480 mg or any other value or range of values therein).

In some embodiments, the present methods employ a thrice daily dosage regimen, and the first daily dose is less than the second and third daily doses. For example, in some embodiments, the second and third daily doses may each independently be from about 5 mg to about 240 mg greater than the first daily dose (e.g., about 5 mg greater, about 10 mg greater, about 15 mg greater, about 20 mg greater, about 25 mg greater, about 30 mg greater, about 35 mg greater, about 40 mg greater, about 45 mg greater, about 50 mg greater, about 55 mg greater, about 60 mg greater, about 65 mg greater, about 70 mg greater, about 75 mg greater, about 80 mg greater, about 85 mg greater, about 90 mg greater, about 95 mg greater, about 100 mg greater, about 105 mg greater, about 110 mg greater, about 115 mg greater, about 120 mg greater, about 125 mg greater, about 130 mg greater, about 135 mg greater, about 140 mg greater, about 145 mg greater, about 150 mg greater, about 155 mg greater, about 160 mg greater, about 165 mg greater, about 170 mg greater, about 175 mg greater, about 180 mg greater, 190 mg, 200 mg, 210 mg, 220 mg, 230 mg, 240 mg or any other value or range of values therein).

In some embodiments, each of the first, second and third doses in a thrice-daily dosing regimen of nalbuphine can be selected as described above, and then titrated upward until the patient experiences satisfactory relief from the pruritic condition. Titrating the dose can include administering a baseline dose, then after a period of observation at the baseline dose value to determine the efficacy and/or side effect severity, of the baseline dose, increasing the dose if the subject does not experience adequate symptom relief. The period of observation at the baseline for any of the first, second and third doses independently can be from about 1 day to about 21 days (e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days). The dose (e.g., any one of three doses in a thrice daily dosing regimen) can be titrated in increments ranging from about 5 mg to about 180 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, or any other value or range of values therein). The second daily dose can be titrated in one or more steps. The amount a dosage is stepped, where there are multiple titration steps, can be the same, or can be different.

In other embodiments, the present methods employ a thrice daily dosage regimen, and the first daily dose is greater than the second and third daily doses. For example, in some embodiments, the first daily dose is from about 5 mg to about 240 mg greater than the second and third daily doses (e.g., about 5 mg greater, about 10 mg greater, about 15 mg greater, about 20 mg greater, about 25 mg greater, about 30 mg greater, about 35 mg greater, about 40 mg greater, about 45 mg greater, about 50 mg greater, about 55 mg greater, about 60 mg greater, about 65 mg greater, about 70 mg greater, about 75 mg greater, about 80 mg greater, about 85 mg greater, about 90 mg greater, about 95 mg greater, about 100 mg greater, about 105 mg greater, about 110 mg greater, about 115 mg greater, about 120 mg greater, about 125 mg greater, about 130 mg greater, about 135 mg greater, about 140 mg greater, about 145 mg greater, about 150 mg greater, about 155 mg greater, about 160 mg greater, about 165 mg greater, about 170 mg greater, about 175 mg greater, about 180 mg greater, about 190 mg greater, about 200 mg greater, about 210 mg greater, about 220 mg greater, about 230 mg greater, about 240 mg greater, or any other value or range of values therein).

In some embodiments, the each of the first, second and third doses in a thrice-daily dosing regimen can be selected as described above, and then titrated upward until the patient experiences satisfactory relief from the pruritic condition. Titrating the dose can include administering a baseline dose, then after a period of observation at the baseline dose value to determine the efficacy of the baseline dose and/or side effect severity, increasing the dose if the subject does not experience adequate symptom relief. The baseline dose may be from about 5 mg to about 240 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 190 mg, about 200 mg, about 210 mg, about 220 mg, about 230 mg, about 240 mg, or any other value or range of values therein).

The titration dose can be in increments ranging from about 5 mg to about 240 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 190 mg, about 200 mg, about 210 mg, about 220 mg, about 230 mg, about 240 mg, or any other value or range of values therein). Each of the first, second and third doses can be titrated in one or more steps. The amount a dosage is stepped, where there are multiple titration steps, can be the same, or can be different. Accordingly, the total daily dose in a thrice daily dosing regimen with a titration dose, including baseline doses and one or more titration doses, may be from about 5 mg to about 480 mg (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 210 mg, about 240 mg, about 270 mg, about 300 mg, about 330 mg, about 360 mg, about 390 mg, about 420 mg, about 450 mg, about 480 mg, or any other value or range of values therein).

Accordingly, the total daily dose in a thrice daily dosing regimen, including baseline dose for each administration and one or more titration doses for each administration independently, may be from about 5 mg a day to about 480 mg a day (e.g., about 15 mg a day, about 20 mg a day, about 25 mg a day, about 30 mg a day, about 35 mg a day, about 40 mg a day, about 45 mg a day, about 50 mg a day, about 55 mg a day, about 60 mg a day, about 65 mg a day, about 70 mg a day, about 75 mg a day, about 80 mg a day, about 85 mg a day, about 90 mg a day, about 95 mg a day, about 100 mg a day, about 105 mg a day, about 110 mg a day, about 115 mg a day, about 120 mg a day, about 125 mg a day, about 130 mg a day, about 135 mg a day, about 140 mg a day, about 145 mg a day, about 150 mg a day, about 155 mg a day, about 160 mg a day, about 165 mg a day, about 170 mg a day, about 175 mg a day, about 180 mg a day, about 185 mg a day, about 190 mg a day, about 195 mg a day, about 200 mg a day, about 205 mg a day, about 210 mg a day, about 215 mg a day, about 220 mg a day, about 225 mg a day, about 230 mg a day, about 235 mg a day, about 240 mg a day, about 245 mg a day, about 250 mg a day, about 255 mg a day, about 260 mg a day, about 265 mg a day, about 270 mg a day, about 275 mg a day, about 280 mg a day, about 285 mg a day, about 290 mg a day, about 295 mg a day, about 300 mg a day, about 305 mg a day, about 310 mg a day, about 315 mg a day, about 320 mg a day, about 325 mg a day, about 330 mg a day, about 335 mg a day, about 340 mg a day, about 345 mg a day, about 350 mg a day, about 355 mg a day, about 360 mg a day, about 390 mg a day, about 420 mg a day, about 450 mg a day, about 480 mg a day, or any other value or range of values therein,).

In some embodiments, a subject, e.g., a human or animal patient can be treated initially with a thrice-daily dosing regimen, and then after titrating either the first daily dose, the second daily dose, and/or the third daily to arrive at a total daily dose effective to treat the patient's pruritus symptoms, the dosage regimen can be changed to either a twice-daily dosage or a once-daily dosage. Each dose of the thrice-daily dosage can be further titrated as described hereinabove to arrive at a therapeutically effective once daily dosage of nalbuphine.

In still other embodiment, the second dose in a thrice-daily dosing regimen may be greater than the first and third doses. In still other embodiments, the third daily dose may be greater than each of the first and second doses. Furthermore, as described above, each of the first, second and third doses in a thrice-daily regimen may be titrated independently of the other two doses, to arrive at a therapeutically efficacious dosing regimen.

In some embodiments, the single daily dosage can be administered in the evening or before bedtime. In other embodiments, the single daily dosage can be administered in the morning. In some embodiments, the single daily dose can be administered around midday (e.g., from about 11 am to about 1 pm). In twice-daily dosing regimens, the two doses may be administered in the morning and evening. In a thrice daily dosing regimen, the three doses may be administered with one dose given in the morning, one dose given at midday and one dose given in the evening.

In some embodiments, the present methods include treating pruritus in a subject, e.g., a human or animal patient, comprising administering to a subject, e.g., a human or animal patient suffering from pruritus an effective amount of a medication consisting of a pharmaceutical compound which is μ-opioid receptor antagonist and a pharmaceutical compound which is a κ-opioid receptor agonist, or pharmaceutically acceptable salts or esters thereof. In certain embodiments, the pharmaceutical compound which is μ-opioid receptor antagonist and the pharmaceutical compound which is a κ-opioid receptor agonist are the same. In some embodiments, the compound is nalbuphine or a pharmaceutically acceptable salt, solvate or ester thereof.

In certain embodiments, the anti-pruritic agent is nalbuphine, and the metabolites include glucuronides (most likely on the phenol and cyclohexane rings), two hydroxylated nalbuphine metabolites (on the cyclobutane ring) and three ketones (hydroxylation of the cyclobutane ring, followed by oxidation to a carbonyl). In some embodiments, the nalbuphine metabolites include nornalbuphine, 6-ketonalbuphine and nalbuphine 3-glucuronide. In some other embodiments, the nalbuphine metabolites include triple hydroxylated nalbuphine, mono-hydroxylated nalbuphine, or mono-glucuronidated nalbuphine or a combination thereof. In certain embodiments, the one or more metabolites of the anti-pruritus agent do not have detectable anti-pruritus activity. In other embodiments, one or more of the metabolites of the anti-pruritus agent exhibit anti-pruritus activity.

In embodiments wherein one or more metabolites of the anti-pruritus agent exhibit anti-pruritus activity, the dosing regimen of the anti-pruritus agent may be adjusted and/or titrated as described hereinabove depending on the clearance rate of the one or more metabolites exhibiting anti-pruritic activity. Such dosage adjustment and/or titration of the dosage of the anti-pruritic agent can be performed to prevent accumulation of either the anti-pruritic agent and/or one or more metabolites, which can also exhibit anti-pruritic activity, to avoid toxicity effects in a patient treated with the present anti-pruritic agent.

In some embodiments, the anti-pruritus agent is completely metabolized (e.g., about 100% metabolized). In other embodiments, the anti-pruritus agent is not completely metabolized (e.g., less than about 100% metabolized). For example, in some embodiments, the anti-pruritus agent is about 100% metabolized, about 95% metabolized, about 90% metabolized, about 85% metabolized, about 80% metabolized, about 75% metabolized, about 70% metabolized, about 65% metabolized, about 60% metabolized, about 55% metabolized, about 50% metabolized, about 45% metabolized, about 40% metabolized, about 35% metabolized, about 25% metabolized, about 20% metabolized, about 15% metabolized, about 10% metabolized, about 5% metabolized, about 1% metabolized, or about 0% metabolized. In certain embodiments, the amount of dialyzable anti-pruritus agent can be measured or monitored by the level of accumulation, e.g., blood plasma level of the anti-pruritus agent or one or more of its metabolites.

In some embodiments, the present methods can further comprise monitoring the plasma concentration of either an anti-pruritic agent, or one or more metabolites thereof. Such monitoring can be performed via measurement of the concentration of the anti-pruritic agent, or one or more metabolites thereof, in the blood of a patient via routine blood testing. Such testing can be conducted at suitable intervals to determine the peak blood plasma concentration of the anti-pruritic agent, or one or more metabolites thereof. Titration of dosage as described hereinabove can also be conducted via blood testing (in addition to qualitative measures described hereinabove for determining therapeutic levels of pruritus relief) to maintain safe levels of the anti-pruritic agent, or one or more metabolites thereof, in the present methods. Once a clearance rate of the anti-pruritic agent, or one or more metabolites thereof is established, an appropriate dosing regimen can be selected to provide target Cₘₐₓ and AUC_{(0-∞)} ranges as described herein above.

According to some embodiments of the present invention, administering of nalbuphine or a pharmaceutically acceptable salt, solvate or ester thereof according to the methods of the present invention provides statistically significant therapeutic effect. In one embodiment, the statistically significant therapeutic effect is determined based on one or more standards or criteria provided by one or more regulatory agencies in the United States, e.g., FDA or other countries. In another embodiment, the statistically significant therapeutic effect is determined based on results obtained from regulatory agency approved clinical trial set up and/or procedure.

In some embodiments, the statistically significant therapeutic effect is determined based on a patient population of at least 100, 200, 300, 400, 500, 600, 700, 800, 900, 1000 or 2000. In some embodiments, the statistically significant therapeutic effect is determined based on data obtained from randomized and double blinded clinical trial set up. In some embodiments, the statistically significant therapeutic effect is determined based on data with a p value of less than or equal to about 0.05, 0.04, 0.03, 0.02 or 0.01. In some embodiments, the statistically significant therapeutic effect is determined based on data with a confidence interval greater than or equal to 95%, 96%, 97%, 98% or 99%. In some embodiments, the statistically significant therapeutic effect is determined on approval of Phase III clinical trial of the methods provided by the present invention, e.g., by FDA in the US.

In some embodiment, the statistically significant therapeutic effect is determined by a randomized double blind clinical trial of a patient population of at least 300 or 350; treated with nalbuphine or a pharmaceutically acceptable salt, solvate or ester thereof and optionally in combination with standard care. In some embodiment, the statistically significant therapeutic effect is determined by a randomized clinical trial of a patient population of at least 300 or 350 and using NRS as primary efficacy parameter and optionally in combination with any other commonly accepted criteria for pruritus assessment.

In general, statistical analysis can include any suitable method permitted by a regulatory agency, e.g., FDA in the US or Europe or any other country. In some embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM) and Cox regression analysis.

The following non-limiting examples illustrate various aspects of the present invention.

### EXAMPLES

### Example 1

A 30 mg extended release (ER) nalbuphine tablet was prepared as follows: Nalbuphine HCl, mannitol, xanthan gum, locust bean gum and calcium sulfate dihydrate were added to a high shear mixer and dried mix at low speed. A granulating solution (water for injection or purified water) was introduced into the mixer at low speed. The wet granulation was granulated at high speed and dried in a fluid bed processor. The dried granules were milled and sized using a conventional mill. The milled granulation was transferred into a diffusion (tumble) mixer. Hydroxypropylcellulose and, when applicable, fumaric acid (180 mg formulations only) were added to the diffusion mixer and blended. Thereafter, magnesium stearate was added to the diffusion mixer and blended. The final blend was compressed using a rotary tablet press.

TABLE 1
30 mg Extended Release Nalbuphine Tablet

| Ingredient | mg/tablet |
|---|---|
| Nalbuphine HCl | 30.0 |
| Mannitol | 108.0 |
| Hydroxypropylcellulose | 35.0 |
| Locust bean gum | 32.4 |
| Xanthan gum | 21.6 |
| Calcium sulfate dehydrate | 18.0 |
| Magnesium stearate | 1.9 |
| Water for injection or Purified water | QS |
| Total: | 246.9 |

The tablet was coated with a non-functional coat (Table 2).

TABLE 2
Nalbuphine HCl ER Tablets, 30 mg Composition

| Component | Tablet (mg/tablet) |
|---|---|
| Nalbuphine HCl | 30.0 |
| Mannitol | 108.0 |
| Hydroxypropylcellulose | 35.0 |
| Locust bean gum | 32.4 |
| Xanthan gum | 21.6 |
| Calcium sulfate dihydrate | 18.0 |
| Magnesium stearate¹ | 1.9 |
| Opadry II White | 7.4 |
| Sterile water for irrigation² | QS |
| Total | 254.3 |

Escalating doses of nalbuphine ER tablets were administered in a Phase I clinical trial (TR01) to renally impaired patients on hemodialysis and healthy subjects with normal kidney function. Specifically, 9 healthy subjects (Cohort 2) and 14 patients undergoing hemodialysis (Cohort 1) were evaluated for possible aquaretic effects of nalbuphine. Healthy subjects and hemodialysis patients received escalating oral doses of nalbuphine every 2-3 days ranging from 30-180 mg BID (9 healthy subjects), 30-180 mg BID (10 hemodialysis patients), or 30-240 mg BID (4 hemodialysis patients) on Study Days 1-15. The presence and extent of a possible aquaretic effect was evaluated by measuring urine output (healthy subjects only) and serum sodium as well as blood pressure and heart rate, as markers of potential volume depletion. Urine specific gravity was also analyzed to assess for reduced urine osmolarity (resulting in a decrease in urine specific gravity) due to free water excretion.

In the dialysis patients, all vital sign measurements and blood samples were collected pre-dialysis. On days of study drug administration, vital signs were measured at 0 (prior to the morning dose), 1.5, 4.5, and 8 hours after the morning dose and 2 hours after the evening meal, coinciding with administration of the PM dose. Blood pressure was also measured at 6 and 10 hours post dinner. On Day −1 (pre-treatment) vital sign measurements were obtained in the morning and evening at approximately the same time that study drug was administered on subsequent treatment days.

Based on historical data, the dialysis patients in TR01 had limited to no residual renal function. Although urine collection was not performed, no adverse event of increased urine output was reported.

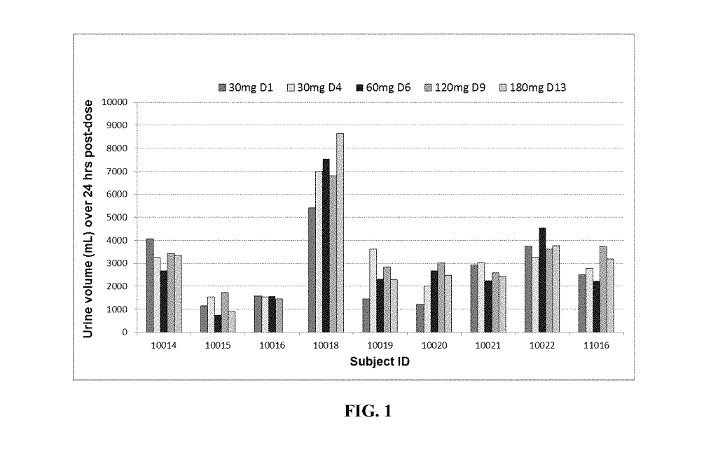

In the healthy subjects, repeated measurements of urine volume, collected during five 24-hour periods, during treatment with progressively higher doses of nalbuphine, showed no evidence of increased urine output related to dose or duration of time on nalbuphine (Table 3 and FIG. 1). Urine specific gravity, measured at Screening and on Study Days −1, 7, and 14, did not decline relative to the lowest pretreatment value (lowest value at Screening or Day −1) in any subject during nalbuphine treatment (Table 4).

Serum sodium was measured at Screening and on Study Days −1, 7, and 14 or 17 and was drawn pre-dialysis in the dialysis patient cohort. No healthy subject (Table 5) or hemodialysis patient (Table 6) developed hypernatremia during nalbuphine treatment nor was there a trend of rising serum sodium during the treatment period in either population.

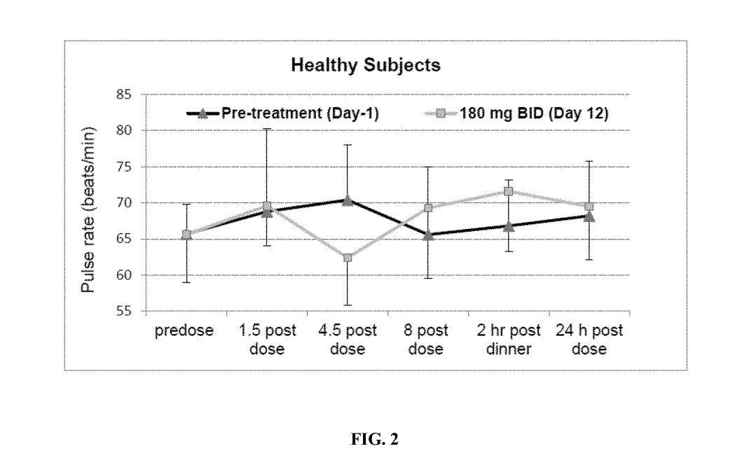

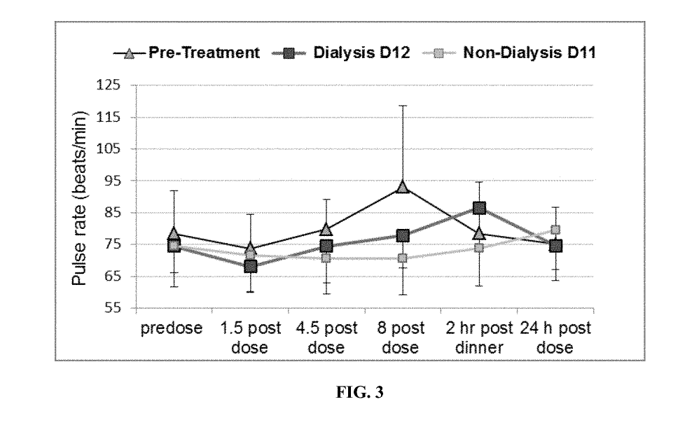

Blood pressure and heart rate were measured at multiple time points on Days −1 through 14 or 17. There were no consistent dose-related reductions in blood pressure in either the healthy subjects (Tables 7-10) or hemodialysis patients (Tables 13-16). Throughout the study, no subjects or patients developed hypotension. The systolic blood pressure remained consistently in the 78-148 mmHg range throughout in the healthy subjects (Table 9) and consistently in the 70-199 mmHg range in the dialysis patients (Table 15). Likewise, heart rate, a sensitive index of volume depletion, remained essentially normal throughout the study with HR ranging from about 50 to about 102 beats/min in the healthy subjects (Table 11) or about 46 to about 126 beats per minute in the dialysis patient cohort (Tables 17), with no evidence of a dose or time-dependent mean heart rate increase in either population (Tables 12 and 18). The mean heart rate over a 24-hour interval prior to treatment (Day −1) and at a dose of 180 mg BID are depicted graphically in FIGS. 2 and 3 for the healthy subjects and hemodialysis patients, respectively.

In conclusion, there is no evidence of an aquaretic effect at nalbuphine doses up to 180 mg BID in healthy subjects and up to 240 mg BID in dialysis patients.

TABLE 3
Urine output over 24 hours post nalbuphine HCl ER tablets by subject as a function
of dose and day in healthy subjects (Cohort 2)

| Dose (mg) | | Total* urine volume (mL) over 24 hours post-dose in subjects ID No. | | | | | | | | |
|---|---|---|---|---|---|---|---|---|---|---|
| AM/PM | Day | 10014 | 10015 | 10016 | 10018 | 10019 | 10020 | 10021 | 10022 | 11016 |
| 30/0 | 1 | 4070 | 1160 | 1570 | 5420 | 1460 | 1210 | 2920 | 3750 | 2495 |
| 30/30 | 4 | 3245 | 1540 | 1540 | 7000 | 3620 | 2015 | 3030 | 3240 | 2770 |
| 60/60 | 6 | 2675 | 750 | 1570 | 7550 | 2315 | 2675 | 2250 | 4540 | 2240 |
| 120/120 | 9 | 3430 | 1720 | 1450 | 6800 | 2845 | 3015 | 2580 | 3615 | 3710 |
| 180/0 | 13 | 3365 | 900 | NA | 8650 | 2275 | 2475 | 2425 | 3770 | 3190 |

*Total volume calculated by adding volume collected over 0-8, 8-12 and 12-24 hours post-dose in TR01 listing 16.2.6.3.1.1

TABLE 4
Urinalysis laboratory test results by subject for healthy subjects (Cohort 2, N = 9)

| Laboratory | | | | | | | | | | |
|---|---|---|---|---|---|---|---|---|---|---|
| Test | | | | | | | | | | |
| (normal | Time | Result in healthy subject ID No. | | | | | | | | |
| range) | Point | 10014 | 10015 | 10016 | 10018 | 10019 | 10020 | 10021 | 10021 | 110016 |
| Glucose | Screening | Negative | Negative | Negative | Negative | Negative | Negative | Negative | Negative | Negative |
| (Negative) | Day −1 | Negative | Negative | Negative | Negative | Negative | Negative | Negative | Negative | Negative |
| | Day 7 | Negative | Negative | Negative | Negative | Negative | Negative | Negative | Negative | Negative |
| | Day 14 | Negative | Negative | Negative | Negative | Negative | Negative | Negative | Negative | Negative* |
| Ketones | Screening | Negative | Negative | Negative | Negative | Negative | Negative | Negative | Trace | Negative |
| (Negative) | Day −1 | Negative | Negative | Negative | Negative | Negative | Negative | Negative | Negative | Negative |
| | Day 7 | Negative | Negative | Negative | Negative | Negative | Negative | Negative | Negative | Negative |
| | Day 14 | Negative | Negative | Negative | Negative | Negative | Negative | Negative | Negative | Negative* |
| Occult Blood | Screening | Negative | Negative | Negative | Negative | Negative | Negative | Negative | Negative | Negative |
| (Negative) | Day −1 | Negative | Negative | Negative | Negative | Negative | Negative | Negative | Negative | Negative |
| | Day 7 | Negative | Not done | Negative | Negative | Negative | Negative | Negative | Negative | Negative |
| | Day 14 | Negative | Negative | Negative | Negative | Negative | Negative | Negative | Negative | Negative* |
| Protein | Screening | Negative | Negative | Negative | Negative | Negative | Negative | Negative | Negative | Negative |
| Negative Or | Day −1 | Negative | Negative | Negative | Negative | Negative | Negative | Negative | Negative | Negative |
| Trace | Day 7 | Negative | Negative | Negative | Negative | Negative | Negative | Negative | Negative | Negative |
| | Day 14 | Negative | Negative | Negative | Negative | Negative | Negative | Negative | Negative | Negative* |
| Specific | Screening | 1.008 | Negative | 1.004 | 1.004 | 1.019 | 1.007 | 1.004 | 1.008 | 1.003 |
| Gravity | Day −1 | 1.013 | Negative | 1.009 | 1.007 | 1.015 | 1.022 | 1.009 | 1.009 | 1.006 |
| 1.001-1.03 | Day 7 | 1.012 | Negative | 1.016 | 1.009 | 1.016 | 1.018 | 1.019 | 1.012 | 1.018 |
| | Day 14 | 1.009 | 1.019 | 1.012 | 1.006 | 1.017 | 1.01 | 1.02 | 1.006 | 1.017* |
| Urobilinogen | Screening | <20 | <20 | <20 | <20 | <20 | <20 | <20 | <20 | <20 |
| (0-19 | Day −1 | <20 | <20 | <20 | <20 | <20 | <20 | <20 | <20 | <20 |
| umol/L) | Day 7 | <20 | Not done | <20 | <20 | <20 | <20 | <20 | <20 | <20 |
| | Day 14 | <20 | <20 | <20 | <20 | <20 | <20 | <20 | <20 | <20* |
| pH (5-8) | Screening | 5.5 | 5 | 5 | 5 | 6 | 5.5 | 5 | 5 | 5 |
| | Day −1 | 5 | 5 | 5 | 5.5 | 5 | 6 | 5 | 5.5 | 5 |
| | Day 7 | 5.5 | Not done | 5.5 | 5 | 5 | 5 | 5 | 6.5 | 5.5 |
| | Day 14 | 5.5 | 5.5 | 5.5 | 5 | 5.5 | 6.5 | 5 | 7 | 6.5* |

Nalbuphine ER dose on Day 7: 60 mg and Day 14: 180 mg.
Source: CSR TR01 Listing 16.2.8.3.1
*early term.

TABLE 5
Individual and mean serum sodium test results in healthy subjects (Cohort 2); normal range = 136-146 mmol/L.

| | Serum sodium (mmol/L) in Subject ID No. | | | | | | | | | | |
|---|---|---|---|---|---|---|---|---|---|---|---|
| | | | | | | | | | | | Mean |
| | | | | | | | | | | Mean | Change from |
| Time point | 10014 | 10015 | 10016 | 10018 | 10019 | 10020 | 10021 | 10021 | 110016 | (SD, n = 9) | Day −1 |
| Screening | 140 | 138 | 140 | 141 | 142 | 142 | 142 | 141 | 146 | 141.3 (2.18) | — |
| Day −1 | 141 | 137 | 140 | 139 | 138 | 142 | 140 | | | 139.8 (1.56) | 0 |
| Day 7 | 142 | 141 | 143 | 141 | 138 | 140 | 140 | 141 | 141 | 140.7 (1.41) | 0.9* |
| | | | | | | | | | | | (1.83) |
| Day 14 | 139 | 139 | 140 | 141 | 140 | 141 | 142 | 143 | 143\hat{ } | 141.0 (1.60) | 1.3* |
| | | | | | | | | | | | (1.75) |

Nalbuphine ER dose on Day 7: 60 mg BID.
Day 14 Discharge day (No dosing).
Last dose on Day 13 = 180 mg.
Day 14: Discharge day . . .
Source: CSR TR01 Listing 16.2.8.2.1 and Table 14.3.4.2.1
\hat{ }early term.
N = 8 (excludes early term subject 110016)

TABLE 6
Individual and mean serum sodium test results in hemodialysis patients (Cohort 1);
normal range = 136-146 mmol/L.

| Time | Serum sodium (mmol/L) in HD subject ID No. | | | | | | | | |
|---|---|---|---|---|---|---|---|---|---|
| point | 10001 | 10002 | 10003 | 10004 | 10005 | 10006 | 10008 | 10009 | 10010 |
| Screen | 142 | 142 | 144 | 136 | 142 | 144 | 138 | 138 | 139 |
| Day −1 | 139 | 143 | 143 | 137 | 143 | 140 | 139 | 137 | 142 |
| Dialysis | | | | | | | | | |
| Day 7 | 133 | 139 | 138 | 139 | 139 | 139 | 137 | 139 | 138 |
| Dialysis | | | | | | | | | |
| Day 141 | 138 | 140 | 139 | 140 | 140 | 141 | 136 | 138 | 137 |
| or 17 | | | | | | | | | |
| Dialysis | | | | | | | | | |

| | Serum sodium (mmol/L) in HD subject ID No. | | | | | | |
|---|---|---|---|---|---|---|---|
| | | | | | | | Change |
| Time | | | | | | Mean | from |
| point | 11007 | 10011 | 10012 | 10013 | 10017 | (SD, n = 13) | Day −1 |
| Screen | 135 | 141 | 142 | 141 | 143 | 140.7 (2.89) | — |
| Day −1 | 136 | 143 | 144 | 140 | 143 | 140.9 (2.84) | 0 |
| Dialysis | | | | | | | |
| Day 7 | 139 | 141 | 141 | 138 | 135 | 138.2 (2.12) | −2.4 |
| Dialysis | | | | | | | (3.16) |
| Day 141 | 139 | 139\hat{ } | 138 | 137 | 134 | 138.2 (1.92) | −2.2 |
| or 17 | | | | | | | (3.54) |
| Dialysis | | | | | | | |

Nalbuphine ER dose on Day 7: 60 mg.
Day 14/Day17: Discharge days (No dosing).
Last dose on Day 13 = 180 mg or Day 15 = 240 mg . . . \hat{ }early term.
N = 13 (excludes early term subject 100011) No dosing.

TABLE 7
Mean and range of diastolic blood pressure (mmHg) as a function of time and day following oral administration of
nalbuphine HCL ER tablets in healthy subjects (N = 9)

| | | DBP (mmHg) mean (SD) and min, max values (N = 9) | | | | | | |
|---|---|---|---|---|---|---|---|---|
| Day/ | Dose (mg) | PRE AM | POST AM DOSE | | | POST DINNER\hat{ } | | |
| Time | AM/PM | DOSE | 1.5 hr | 4.5 hr | 8 hr | 2 hr^{§} | 6 hr | 10 hr |
| Day−1 | 0 | 69.6 (9.93) | 73.0 (8.51) | 72.6 (8.50) | 76.3 (9.58) | 72.8 (9.48) | 71.6 (12.66) | 74.3 (9.76) |
| | | 54, 80 | 65, 93 | 63, 84 | 64, 91 | 58, 93 | 50, 95 | 60, 89 |
| Day 1 | 30/0 | 76.0 (8.50) | 70.4 (5.59) | 73.1 (4.99) | 71.2 (7.46) | 71.4 (6.77) | 69.1 (11.27) | 65.2 (5.78) |
| | | 68, 92 | 64, 83 | 65, 79 | 59, 85 | 62, 83 | 57, 91 | 55, 73 |
| Day 2 | 30/30 | 74.0 (8.73) | 71.1 (6.95) | 74.2 (7.56) | 73.8 (8.18) | 75.9 (6.43) | 68.2 (8.35) | 73.2 (7.03) |
| | | 62, 91 | 63, 86 | 64, 88 | 64, 88 | 68, 84 | 53, 76 | 64, 83 |
| Day 3 | 30/30 | 74.7 (7.33) | 71.3 (7.09) | 74.7 (9.30) | 75.1 (9.79) | 75.3 (11.03) | 67.3 (11.47) | 63.6 (10.71) |
| | | 66, 85 | 61, 81 | 57, 87 | 60, 91 | 61, 98 | 42, 81 | 39, 74 |
| Day 4 | 30/60 | 75.7 (9.22) | 73.1 (9.65) | 74.0 (9.62) | 71.7 (6.98) | 74.9 (7.83) | 69.7 (11.10) | 67.7 (7.89) |
| | | 65, 91 | 63, 93 | 59, 89 | 62, 84 | 64, 90 | 49, 83 | 53, 76 |
| Day 5 | 60/60 | 73.9 (8.52) | 74.3 (6.61) | 72.7 (4.64) | 74.3 (8.09) | 75.6 (8.50) | 66.9 (7.69) | 64.7 (10.11) |
| | | 65, 94 | 64, 84 | 65, 79 | 58, 84 | 59, 88 | 57, 79 | 45, 79 |
| Day 6 | 60/60 | 75.8 (8.14) | 70.1 (5.28) | 73.8 (8.73) | 71.3 (7.04) | 76.9 (6.27) | 65.2 (8.47) | 65.9 (7.98) |
| | | 63, 87 | 64, 80 | 58, 92 | 55, 81 | 70, 87 | 48, 75 | 51, 76 |
| Day 7 | 60/120 | 76.9 (8.24) | 68.9 (4.40) | 71.7 (8.57) | 75.4 (9.32) | 72.0 (7.28) | 66.2 (7.98) | 66.8 (10.21) |
| | | 66, 91 | 63, 75 | 58, 83 | 59, 92 | 58, 81 | 56, 77 | 52, 90 |
| Day 8 | 120/120 | 72.9 (8.67) | 72.6 (6.62) | 72.7 (8.22) | 72.0 (9.43) | 73.0 (6.96) | 72.3 (7.30) | 66.8 (3.15) |
| | | 57, 82 | 61, 80 | 56, 84 | 59, 88 | 62, 85 | 62, 83 | 63, 73 |
| Day 9 | 120/120 | 75.7 (8.31) | 71.0 (7.79) | 71.9 (7.79) | 72.7 (9.73) | 76.6 (8.44) | 71.4 (8.35) | 64.1 (8.91) |
| | | 59, 88 | 57, 84 | 60, 83 | 55, 83 | 63, 88 | 63, 87 | 45, 74 |
| Day 10 | 120/180 | 74.4 (8.69) | 70.3 (8.09) | 78.2 (9.05) | 75.7 (7.65) | 72.0 (7.50) | 68.4 (11.52) | 64.8 (8.80) |
| | | 57, 85 | 53, 80 | 59, 90 | 63, 85 | 58, 81 | 50, 82 | 44, 74 |
| Day 11 | 180/180 | 76.0 (7.25) | 72.1 (9.97) | 71.2 (8.74) | 73.6 (8.43) | 74.8 (6.76) | 68.8 (9.71) | 67.6 (12.34) |
| | | 66, 87 | 61, 91 | 56, 85 | 61, 86 | 68, 88 | 52, 80 | 48, 88 |
| Day 12 | 180/180 | 74.5 (7.86) | 73.0 (6.76) | 76.3 (7.21) | 76.5 (9.46) | 76.4 (7.80) | 66.0 (9.20) | 66.8 (8.86) |
| | | 63, 83 | 66, 86 | 66, 86 | 60, 89 | 64, 86 | 50, 83 | 57, 82 |
| Day 13 | 180/0 | 73.6 (11.43) | 71.1 (5.82) | 74.0 (6.48) | 74.6 (8.73) | 73.9 (9.19) | 64.6 (5.85) | 66.6 (7.25) |
| | | 52, 91 | 63, 78 | 65, 82 | 62, 87 | 56, 86 | 55, 74 | 53, 76 |
| Day 14 | 0 | 73.9 (7.79) | | | | | | |
| Discharge | | 63, 83 | | | | | | |

\hat{ }Dinner about 10 hours post-am dose.
^{§}Coincides with evening dose administration

TABLE 8
Mean and range of change from baseline in diastolic blood pressure (mmHg) as a function of dose, time and day following oral
administration of nalbuphine HCL ER tablets in healthy subjects (N = 9)

| | | DBP Change from baseline (mmHg) mean (SD) and min, max values (N = 9) | | | | | | |
|---|---|---|---|---|---|---|---|---|
| Day/ | Dose (mg) | PRE AM | POST AM DOSE | | | POST DINNER\hat{ } | | |
| Time | AM/PM | DOSE | 1.5 hr | 4.5 hr | 8 hr | 2 hr^{§} | 6 hr | 10 hr |
| Day 1 | 30/0 | 0 | −5.6 (6.33) | −2.9 (7.56) | −4.8 (4.97) | −4.6 (6.97) | −6.9 (5.64) | −10.8 (6.82) |
| | | Baseline | −19, 3 | −15, 11 | −13, 1 | −17, 4 | −15, −1 | −20, −3 |
| Day 2 | 30/30 | −2.0 (4.00) | −4.9 (4.43) | −1.8 (6.91) | −2.2 (6.65) | −0.1 (6.57) | −7.8 (9.63) | −2.8 (8.29) |
| | | −9, 3 | −12, 0 | −16, 6 | −8, 12 | −11, 12 | −19, 7 | −10, 12 |
| Day 3 | 30/30 | −1.3 (6.04) | −4.7 (5.81) | −1.3 (7.31) | −0.9 (11.34) | −0.7 (8.83) | −8.7 (10.21) | −12.4 (13.58) |
| | | −8, 12 | −11, 5 | −15, 11 | −19, 18 | −14, 10 | −33, 1 | −36, 6 |
| Day 4 | 30/60 | −0.3 (5.94) | −2.9 (6.53) | −2.0 (7.57) | −4.3 (6.42) | −1.1 (4.34) | −6.3 (14.27) | −8.3 (11.10) |
| | | −7, 11 | −12, 8 | −13, 11 | −10, 9 | −5, 7 | −28, 15 | −23, 7 |
| Day 5 | 60/60 | −2.1 (6.83) | −1.7 (6.50) | −3.3 (6.36) | −1.7 (7.02) | −0.4 (7.30) | −9.1 (9.39) | −11.3 (10.02) |
| | | −17, 7 | −12, 10 | −14, 5 | −14, 9 | −13, 7 | −21, 3 | −30, 2 |
| Day 6 | 60/60 | −0.2 (5.80) | −5.9 (7.29) | −2.2 (7.61) | −4.7 (9.35) | 0.9 (4.68) | −10.8 (10.77) | −10.1 (9.62) |
| | | −9, 7 | −21, 4 | −15, 8 | −20, 6 | −7, 6 | −24, 7 | −21, 4 |
| Day 7 | 60/120 | 0.9 (6.81) | −7.1 (8.37) | −4.3 (6.22) | −0.6 (7.25) | −4.0 (6.80) | −9.8 (9.47) | −9.2 (10.70) |
| | | −13, 9 | −21, 3 | −14, 6 | −13, 8 | −14, 5 | −20, 6 | −21, 13 |
| Day 8 | 120/120 | −3.1 (7.66) | −3.4 (10.93) | −3.3 (7.65) | −4.0 (9.31) | −3.0 (5.45) | −3.7 (6.48) | −9.2 (9.58) |
| | | −15, 7 | −22, 10 | −16, 7 | −16, 16 | −10, 4 | −16, 6 | −25, 1 |
| Day 9 | 120/120 | −0.3 (8.02) | −5.0 (7.07) | −4.1 (6.94) | −3.3 (7.84) | 0.6 (7.73) | −4.6 (6.75) | −11.9 (9.82) |
| | | −13, 14 | −16, 3 | −12, 6 | −17, 8 | −10, 11 | −13, 10 | −30, 2 |
| Day 10 | 120/180 | −1.6 (9.02) | −5.7 (9.60) | 2.2 (10.74) | −0.3 (8.35) | −4.0 (8.65) | −7.6 (10.30) | −11.2 (11.53) |
| | | −15, 9 | −21, 3 | −13, 15 | −12, 10 | −14, 13 | −25, 6 | −27, 0 |
| Day 11 | 180/180 | 0.0 (8.73) | −3.9 (7.52) | −4.8 (8.47) | −2.4 (8.28) | −1.2 (6.02) | −7.2 (9.19) | −8.4 (8.88) |
| | | −18, 9 | −14, 9 | −16, 5 | −12, 10 | −10, 10 | −23, 3 | −20, 10 |
| Day 12 | 180/180 | −1.6 (5.55) | −3.1 (8.61) | 0.1 (5.84) | 0.4 (11.08) | 0.3 (4.68) | −10.1 (11.00) | −9.4 (10.38) |
| | | −11, 6 | −22, 8 | −7, 6 | −13, 21 | −7, 6 | −23, 6 | −20, −3 |
| Day 13 | 180/0 | −2.5 (11.26) | −5.0 (6.50) | −2.1 (5.77) | −1.5 (7.62) | −2.3 (7.07) | −11.5 (9.55) | −9.5 (6.74) |
| | | −20, 14 | −16, 5 | −10, 7 | −11, 10 | −16, 6 | −28, −3 | −20, −3 |
| Day 14 | 0 | −2.3 (8.89) | — | — | — | — | | — |
| Discharge | | −15, 9 | | | | | | |

\hat{ }Dinner about 10 hours post-am dose.
^{§}Coincides with evening dose administration

TABLE 9
Mean and range of systolic blood pressure (mmHg) as a function of dose, time and day following oral administration of
nalbuphine HCL ER tablets in healthy subjects (N = 9)

| | | SBP (mmHg) mean (SD) and min, max values (N = 9) | | | | | | |
|---|---|---|---|---|---|---|---|---|
| Day/ | Dose (mg) | PRE AM | POST AM DOSE | | | POST DINNER\hat{ } | | |
| Time | AM/PM | DOSE | 1.5 hr | 4.5 hr | 8 hr | 2 hr^{§} | 6 hr | 10 hr |
| Day−1 | 0 | 110.3 (9.01) | 118.0 (12.99) | 120.7 (8.50) | 125.4 (13.37) | 118.7 (8.94) | 116.3 (10.93) | 118.4 (11.54) |
| | | 97, 119 | 103, 146 | 109, 139 | 108, 144 | 108, 134 | 103, 134 | 105, 142 |
| Day 1 | 30/0 | 118.3 (8.87) | 116.0 (6.32) | 114.4 (5.53) | 114.3 (5.29) | 119.4 (5.92) | 116.1 (11.38) | 107.8 (6.91) |
| | | 107, 139 | 106, 127 | 103, 120 | 107, 123 | 110, 128 | 102, 139 | 98, 117 |
| Day 2 | 30/30 | 118.2 (9.48) | 116.0 (9.04) | 116.6 (9.23) | 118.8 (11.80) | 123.1 (10.40) | 119.2 (9.54) | 115.0 (12.68) |
| | | 102, 132 | 99, 127 | 102, 134 | 101, 142 | 107, 133 | 106, 135 | 95, 141 |
| Day 3 | 30/30 | 117.8 (10.89) | 115.0 (8.44) | 117.3 (9.25) | 120.7 (8.25) | 116.9 (7.01) | 114.0 (16.45) | 113.1 (15.09) |
| | | 99, 132 | 99, 125 | 103, 132 | 114, 141 | 105, 131 | 78, 131 | 86, 140 |
| Day 4 | 30/60 | 120.7 (11.75) | 114.0 (11.91) | 116.4 (8.32) | 117.2 (9.15) | 122.1 (9.09) | 119.4 (14.31) | 114.4 (14.08) |
| | | 109, 144 | 94, 133 | 99, 127 | 106, 138 | 110, 134 | 96, 140 | 96, 135 |
| Day 5 | 60/60 | 117.9 (6.49) | 121.0 (10.52) | 114.0 (10.56) | 117.7 (6.71) | 118.3 (11.26) | 110.9 (11.53) | 109.6 (15.36) |
| | | 109, 128 | 106, 137 | 99, 136 | 107, 128 | 105, 133 | 92, 127 | 86, 132 |
| Day 6 | 60/60 | 121.4 (8.85) | 117.8 (14.47) | 114.0 (7.57) | 115.0 (10.21) | 119.2 (9.00) | 117.8 (13.89) | 116.7 (10.37) |
| | | 110, 135 | 98, 139 | 101, 123 | 102, 132 | 101, 128 | 94, 135 | 106, 137 |
| Day 7 | 60/120 | 118.4 (15.47) | 117.1 (8.19) | 114.1 (10.31) | 118.0 (12.36) | 120.7 (10.68) | 116.1 (9.82) | 110.4 (11.35) |
| | | 96, 141 | 104, 128 | 99, 128 | 98, 138 | 108, 142 | 106, 131 | 93, 134 |
| Day 8 | 120/120 | 115.1 (9.65) | 112.6 (7.52) | 116.6 (7.72) | 123.7 (11.79) | 126.3 (9.96) | 121.7 (10.79) | 110.0 (8.19) |
| | | 100, 126 | 98, 120 | 105, 129 | 100, 140 | 113, 138 | 106, 143 | 95, 122 |
| Day 9 | 120/120 | 122.8 (8.94) | 116.8 (12.69) | 112.9 (11.32) | 123.9 (12.61) | 121.9 (9.77) | 121.6 (11.88) | 109.9 (9.18) |
| | | 109, 134 | 100, 133 | 100, 132 | 106, 143 | 110, 140 | 101, 142 | 95, 122 |
| Day 10 | 120/180 | 123.8 (12.85) | 116.6 (7.09) | 119.4 (11.42) | 125.1 (6.19) | 120.9 (9.33) | 121.2 (14.63) | 109.2 (9.47) |
| | | 99, 141 | 108, 132 | 99, 137 | 117, 135 | 110, 136 | 102, 148 | 90, 119 |
| Day 11 | 180/180 | 121.8 (12.40) | 118.6 (14.70) | 113.1 (7.54) | 116.8 (10.51) | 124.3 (13.53) | 120.0 (11.02) | 113.8 (9.48) |
| | | 96, 137 | 102, 140 | 99, 125 | 100, 135 | 108, 148 | 100, 138 | 100, 129 |
| Day 12 | 180/180 | 127.5 (11.59) | 116.3 (11.71) | 112.9 (10.47) | 127.3 (11.36) | 124.3 (6.45) | 112.9 (17.47) | 110.5 (6.21) |
| | | 109, 143 | 103, 132 | 100, 134 | 107, 139 | 113, 134 | 89, 145 | 102, 118 |
| Day 13 | 180/0 | 114.1 (9.72) | 118.1 (9.26) | 118.8 (8.97) | 127.5 (9.09) | 121.8 (6.61) | 114.9 (10.79) | 112.9 (10.58) |
| | | 98, 127 | 107, 130 | 105, 133 | 114, 139 | 111, 130 | 99, 126 | 99, 128 |
| Day 14 | 0 | 116.6 (9.36) | — | — | — | — | — | — |
| Discharge | | 106, 131 | | | | | | |

\hat{ }Dinner administered about 10 hours post-am dose.
^{§}Coincides with evening dose was administered

TABLE 10
Mean and range of change from baseline in systolic blood pressure (mmHg) as a function of dose, time and day following
oral administration of nalbuphine HCL ER tablets in healthy subjects (N = 9)

| | | SBP Change from baseline (mmHg) mean (SD) and min, max values (N = 9) | | |
|---|---|---|---|---|
| Day/ | Dose (mg) | PRE AM | POST AM DOSE | POST DINNER\hat{ } |

| | | | | | | | | |
|---|---|---|---|---|---|---|---|---|
| Time | AM/PM | DOSE | 1.5 hr | 4.5 hr | 8 hr | 2 hr^{§} | 6 hr | 10 hr |

| | | | | | | | | |
|---|---|---|---|---|---|---|---|---|
| Day 1 | 30/0 | 0 | −2.3 (6.20) | −3.9 (7.27) | −4.0 (5.85) | 1.1 (6.49) | −2.2 (12.37) | −10.6 (7.32) |
| | | 0 | −13, 5 | −21, 3 | −16, 2 | −12, 7 | −23, 22 | −27, 0 |
| Day 2 | 30/30 | −0.1 (8.77) | −2.3 (9.39) | −1.8 (7.64) | 0.4 (6.69) | 4.8 (11.17) | 0.9 (8.46) | −3.3 (7.42) |
| | | −14, 15 | −20, 12 | −13, 9 | −10, 14 | −10, 19 | −8, 18 | −17, 8 |
| Day 3 | 30/30 | −0.6 (8.25) | −3.3 (8.43) | −1.0 (9.96) | 2.3 (11.08) | −1.4 (4.25) | −4.3 (12.91) | −5.2 (14.13) |
| | | −8, 14 | −14, 10 | −14, 15 | −22, 20 | −8, 3 | −34, 8 | −26, 19 |
| Day 4 | 30/60 | 2.3 (8.92) | −4.3 (9.35) | −1.9 (9.93) | −1.1 (5.88) | 3.8 (10.37) | 1.1 (13.31) | −3.9 (10.52) |
| | | −9, 19 | −13, 16 | −17, 13 | −11, 6 | −11, 19 | −21, 23 | −21, 16 |
| Day 5 | 60/60 | −0.4 (7.20) | 2.7 (8.28) | −4.3 (13.71) | −0.7 (7.28) | 0.0 (9.04) | −7.4 (10.85) | −8.8 (11.53) |
| | | −11, 11 | −10, 12 | −24, 24 | −11, 9 | −11, 14 | −20, 10 | −29, 3 |
| Day 6 | 60/60 | 3.1 (12.28) | −0.6 (15.34) | −4.3 (7.45) | −3.3 (9.07) | 0.9 (10.46) | −0.6 (13.11) | − 1.7 (11.15) |
| | | −13, 28 | −19, 27 | −16, 6 | −15, 12 | −15, 11 | −22, 23 | −11, 25 |
| Day 7 | 60/120 | 0.1 (15.93) | −1.2 (11.79) | −4.2 (12.35) | −0.3 (12.27) | 2.3 (5.85) | −2.2 (8.50) | −7.9 (8.24) |
| | | −20, 29 | −17, 16 | −20, 16 | −18, 26 | −5, 15 | −11, 14 | −24, 3 |
| Day 8 | 120/120 | −3.2 (11.07) | −5.8 (10.65) | −1.8 (7.68) | 5.3 (12.99) | 8.0 (11.90) | 3.3 (12.53) | −8.3 (10.27) |
| | | −23, 11 | −21, 11 | −11, 10 | −15, 23 | −4, 23 | −16, 26 | −25, 7 |
| Day 9 | 120/120 | 4.4 (8.89) | −1.6 (16.74) | −5.4 (13.57) | 5.6 (13.54) | 3.6 (7.91) | 3.2 (14.71) | −8.4 (9.75) |
| | | −7, 19 | −20, 21 | −20, 20 | −10, 27 | −7, 16 | −20, 25 | −21, 5 |
| Day 10 | 120/180 | 5.4 (15.15) | −1.8 (4.68) | 1.1 (14.67) | 6.8 (10.35) | 2.6 (11.25) | 2.9 (12.31) | −9.1 (8.74) |
| | | −17, 28 | −8, 6 | −18, 25 | −15, 18 | −12, 22 | −9, 31 | −22, 2 |
| Day 11 | 180/180 | 3.4 (12.60) | 0.2 (13.45) | −5.2 (11.16) | −1.6 (11.98) | 6.0 (9.73) | 1.7 (12.25) | −4.6 (8.59) |
| | | −21, 20 | −18, 28 | −22, 8 | −21, 18 | −8, 27 | −16, 21 | −13, 12 |
| Day 12 | 180/180 | 8.4 (10.86) | −2.9 (9.28) | −6.3 (10.75) | 8.1 (11.62) | 5.1 (10.38) | −6.3 (15.65) | −8.6 (8.25) |
| | | −3, 26 | −13, 11 | −21, 17 | −9, 22 | −17, 17 | −28, 28 | −22, 3 |
| Day 13 | 180/0 | −5.0 (8.16) | −1.0 (11.07) | −0.4 (10.64) | 8.4 (14.73) | 2.6 (11.11) | −4.3 (12.31) | −6.3 (10.39) |
| | | −18, 5 | −14, 18 | −11, 16 | −16, 22 | −20, 13 | −18, 14 | |
| Day 14 | 0 | −2.5 (8.93) | — | — | — | — | — | — |
| Discharge | | −14, 14 | | | | | | |

\hat{ }Dinner about 10 hours post-am dose.
^{§}Coincides with evening dose administration

TABLE 11
Mean heart rate (HR) as a function of dose, time and day following oral administration
of nalbuphine HCL ER tablets in healthy subjects (N = 9)

| | | HR (beats/min) mean (SD) and min, max values (N = 9) | | | | |
|---|---|---|---|---|---|---|
| | | | | | | POST |
| Day/ | Dose (mg) | | POST AM DOSE | | | DINNER\hat{ } |
| Time | AM/PM | PRE AM DOSE | 1.5 hr | 4.5 hr | 8 hr | 2 hr^{§} |
| Day−1 | 0 | 65.7 (4.11) | 68.8 (11.48) | 70.4 (7.63) | 65.6 (9.37) | 66.8 (6.44) |
| | | 61, 72 | 55, 83 | 57, 81 | 53, 81 | 54, 73 |
| Day 1 | 30/0 | 68.2 (7.55) | 68.2 (6.82) | 61.7 (6.56) | 63.4 (6.69) | 67.4 (9.18) |
| | | 59, 85 | 56, 78 | 50, 72 | 51, 73 | 54, 86 |
| Day 2 | 30/30 | 66.7 (7.25) | 68.7 (5.85) | 65.8 (8.20) | 65.4 (6.02) | 67.2 (7.36) |
| | | 56, 78 | 59, 80 | 54, 75 | 54, 72 | 58, 78 |
| Day 3 | 30/30 | 66.6 (6.69) | 67.0 (7.26) | 64.0 (4.80) | 72.8 (7.74) | 70.4 (10.65) |
| | | 55, 76 | 57, 80 | 56, 71 | 62, 86 | 57, 93 |
| Day 4 | 30/60 | 70.2 (9.31) | 71.1 (7.79) | 64.7 (6.14) | 68.1 (6.03) | 72.4 (8.41) |
| | | 57, 82 | 57, 80 | 55, 73 | 60, 79 | 59, 86 |
| Day 5 | 60/60 | 66.8 (8.79) | 71.3 (8.40) | 61.7 (5.63) | 66.4 (6.56) | 69.4 (8.19) |
| | | 55, 82 | 62, 82 | 54, 68 | 55, 76 | 57, 83 |
| Day 6 | 60/60 | 68.4 (6.33) | 70.0 (8.62) | 62.3 (6.95) | 68.4 (7.86) | 71.9 (7.29) |
| | | 60, 80 | 61, 83 | 54, 76 | 54, 77 | 62, 86 |
| Day 7 | 60/120 | 71.2 (12.56) | 70.4 (7.84) | 62.6 (7.89) | 70.4 (9.25) | 71.1 (8.30) |
| | | 58, 101 | 59, 79 | 54, 75 | 60, 90 | 62, 90 |
| Day 8 | 120/120 | 68.0 (8.15) | 66.6 (9.53) | 61.2 (8.96) | 65.3 (5.87) | 70.6 (7.42) |
| | | 57, 84 | 55, 84 | 51, 76 | 57, 76 | 60, 81 |
| Day 9 | 120/120 | 68.4 (8.89) | 69.2 (9.92) | 62.9 (7.88) | 63.6 (6.31) | 74.8 (11.88) |
| | | 57, 83 | 56, 82 | 54, 78 | 56, 73 | 58, 102 |
| Day 10 | 120/180 | 71.3 (10.17) | 69.9 (8.01) | 62.3 (10.50) | 70.9 (5.60) | 71.1 (7.75) |
| | | 60, 88 | 61, 85 | 45, 79 | 62, 80 | 62, 84 |
| Day 11 | 180/180 | 66.4 (6.23) | 68.0 (8.00) | 62.6 (6.86) | 69.0 (8.96) | 70.7 (8.86) |
| | | 56, 80 | 57, 77 | 54, 72 | 55, 86 | 58, 85 |
| Day 12 | 180/180 | 65.6 (6.63) | 69.6 (5.53) | 62.4 (6.57) | 69.3 (9.75) | 71.6 (8.35) |
| | | 55, 75 | 61, 77 | 55, 71 | 56, 86 | 60, 86 |
| Day 13 | 180/0 | 69.5 (7.37) | 69.8 (8.68) | 63.3 (6.02) | 71.8 (9.25) | 72.3 (10.00) |
| | | 60, 82 | 61, 86 | 55, 71 | 59, 87 | 59, 90 |
| Day 14 | 0 | 66.9 (10.27) | — | — | — | — |
| Discharge | | 55, 85 | | | | |

\hat{ }Dinner about 10 hours post-am dose.
^{§}Coincides with evening dose administration

TABLE 12
Mean and range of change in heart rate as a function of dose, time and day following
oral administration of nalbuphine HCL ER tablets in healthy subjects (N = 9)

| | | HR change from baseline (beats/min) | | | | |
|---|---|---|---|---|---|---|
| | | mean (SD) and min, max values (N = 9) | | | | |
| | | | | | | POST |
| Day/ | Dose (mg) | PRE AM | POST AM DOSE | | | DINNER |
| Time | AM/PM | DOSE | 1.5 hr | 4.5 hr | 8 hr | 2 hr^{§} |
| Day 1 | 30/0 | 0 | 0.0 (6.14) | −6.6 (7.68) | −4.8 (5.89) | −0.8 (9.09) |
| | | Baseline | −13, 9 | −23, 3 | −17, 4 | −15, 17 |
| Day 2 | 30/30 | −1.6 (7.38) | 0.4 (8.20) | −2.4 (9.68) | −2.8 (7.14) | −1.0 (9.73) |
| | | −18, 9 | −18, 11 | −23, 6 | −13, 12 | −24, 9 |
| Day 3 | 30/30 | −1.7 (7.47) | −1.2 (10.10) | −4.2 (6.80) | 4.6 (11.15) | 2.2 (11.68) |
| | | −19, 7 | −26, 11 | −19, 2 | −17, 18 | −18, 25 |
| Day 4 | 30/60 | 2.0 (10.81) | 2.9 (11.47) | −3.6 (8.75) | −0.1 (10.07) | 4.2 (10.88) |
| | | −23, 14 | −25, 12 | −25, 5 | −24, 10 | −19, 17 |
| Day 5 | 60/60 | −1.4 (9.36) | 3.1 (12.70) | −6.6 (9.17) | −1.8 (9.71) | 1.2 (11.57) |
| | | −19, 14 | −23, 20 | −29, 1 | −23, 10 | −23, 15 |
| Day 6 | 60/60 | 0.2 (9.39) | 1.8 (13.11) | −5.9 (8.64) | 0.2 (11.85) | 3.7 (9.80) |
| | | −19, 12 | −23, 23 | −23, 8 | −28, 10 | −17, 18 |
| Day 7 | 60/120 | 3.0 (13.11) | 2.2 (12.59) | −5.7 (10.90) | 2.2 (13.41) | 2.9 (11.24) |
| | | −18, 33 | −26, 16 | −30, 7 | −25, 22 | −21, 22 |
| Day 8 | 120/120 | −0.2 (10.02) | −1.7 (12.80) | −7.0 (10.43) | −2.9 (11.21) | 2.3 (11.77) |
| | | −19, 16 | −27, 16 | −24, 10 | −28, 8 | −21, 21 |
| Day 9 | 120/120 | 0.2 (10.89) | 1.0 (13.07) | −5.3 (10.23) | −4.7 (9.00) | 6.6 (13.87) |
| | | −22, 13 | −29, 14 | −28, 10 | −24, 4 | −15, 34 |
| Day 10 | 120/180 | 3.1 (12.59) | 1.7 (10.67) | −5.9 (10.96) | 2.7 (8.12) | 2.9 (11.56) |
| | | −24, 20 | −22, 17 | −23, 11 | −13, 12 | −23, 15 |
| Day 11 | 180/180 | −1.8 (8.09) | −0.2 (11.07) | −5.7 (8.37) | 0.8 (12.03) | 2.4 (12.66) |
| | | −19, 12 | −28, 9 | −23, 4 | −25, 18 | −27, 17 |
| Day 12 | 180/180 | −2.1 (8.90) | 1.9 (9.20) | −5.4 (9.18) | 1.5 (12.54) | 3.9 (10.52) |
| | | −18, 6 | −18, 11 | −26, 3 | −22, 18 | −15, 18 |
| Day 13 | 180/0 | 1.8 (6.41) | 2.0 (10.60) | −4.5 (7.11) | 4.0 (11.70) | 4.5 (12.76) |
| | | −6, 14 | −18, 18 | −19, 3 | −15, 21 | −18, 22 |
| Day 14 | 0 | −0.9 (11.23) | — | — | — | — |
| Discharge | | −17, 17 | | | | |

\hat{ }Dinner about 10 hours post-am dose.
^{§}Coincides with evening dose administration

TABLE 13
Mean and range of diastolic blood pressure (mmHg) as a function of dose, time and day following oral administration of
nalbuphine HCL ER tablets in hemodialysis patients (N = 14)

| | | DBP (mmHg) mean (SD) and min, max values (N = 14) in HD patients | | | | | | |
|---|---|---|---|---|---|---|---|---|
| Day/ | Dose (mg) | PRE AM | POST AM DOSE | | | POST DINNER | | |
| Time | AM/PM | DOSE | 1.5 hr | 4.5 hr | 8 hr | 2 hr^{§} | 6 hr | 10 hr |
| Day−1 | 0 | 76.4 (7.74) | 75.5 (7.94) | 85.5 (5.92) | 78.0 (15.03) | 69.8 (11.09) | 68.3 (13.72) | 64.3 (16.66) |
| Dialysis | | 67, 96 | 64, 82 | 77, 90 | 56, 89 | 57, 84 | 51, 84 | 40, 78 |
| Day 1 | 30/0 | 74.1 (8.46) | 69.9 (9.43) | 72.1 (10.20) | 74.9 (9.33) | 76.8 (10.94) | 77.3 (11.82) | 71.9 (11.90) |
| | | 56, 86 | 55, 92 | 53, 89 | 56, 92 | 52, 96 | 59, 107 | 53, 89 |
| Day 2 | 30/30 | 78.1 (11.36) | 71.6 (8.24) | 76.7 (10.48) | 81.5 (9.78) | 80.5 (9.10) | 81.0 (10.70) | 75.4 (10.47) |
| | | 52, 94 | 54, 83 | 60, 94 | 65, 98 | 61, 94 | 61, 98 | 59, 90 |
| Day 3 | 30/30 | 84.7 (9.01) | 80.9 (8.45) | 82.0 (9.60) | 71.3 (11.53) | 71.1 (10.37) | 73.3 (12.94) | 71.1 (10.33) |
| Dialysis | | 67, 96 | 66, 97 | 64, 98 | 50, 95 | 50, 92 | 46, 94 | 48, 89 |
| Day 4 | 30/60 | 76.5 (9.16) | 72.9 (6.43) | 76.9 (9.01) | 75.4 (11.28) | 79.6 (10.18) | 79.1 (12.42) | 73.6 (17.13) |
| | | 62, 93 | 64, 86 | 63, 90 | 48, 92 | 54, 92 | 63, 105 | 47, 105 |
| Day 5 | 60/60 | 81.0 (11.66) | 76.1 (5.54) | 79.9 (10.47) | 71.1 (5.99) | 74.6 (11.61) | 73.4 (7.86) | 70.4 (13.26) |
| Dialysis | | 58, 100 | 63, 84 | 65, 100 | 62, 81 | 55, 98 | 61, 85 | 48, 88 |
| Day 6 | 60/60 | 76.1 (11.32) | 72.7 (7.95) | 76.0 (8.63) | 76.0 (9.62) | 79.9 (8.90) | 77.7 (14.22) | 76.0 (11.88) |
| | | 55, 97 | 59, 86 | 62, 92 | 62, 88 | 67, 97 | 44, 105 | 51, 90 |
| Day 7 | 60/120 | 81.5 (11.06) | 75.6 (10.73) | 80.9 (10.03) | 70.6 (8.44) | 73.6 (11.78) | 72.9 (9.81) | 70.8 (9.62) |
| Dialysis | | 63, 96 | 56, 87 | 64, 96 | 59, 85 | 54, 92 | 61, 87 | 54, 84 |
| Day 8 | 120/120 | 75.5 (11.80) | 69.6 (10.61) | 73.6 (8.04) | 72.2 (12.64) | 77.1 (9.11) | 76.3 (12.16) | 74.9 (13.40) |
| | | 56, 100 | 52, 85 | 59, 87 | 45, 93 | 55, 88 | 54, 97 | 53, 96 |
| Day 9 | 120/120 | 79.4 (14.71) | 77.7 (11.21) | 77.2 (9.48) | 79.9 (8.01) | 83.6 (7.25) | 80.9 (13.11) | 77.6 (13.51) |
| | | 48, 97 | 54, 92 | 60, 93 | 68, 92 | 73, 94 | 64, 109 | 53, 98 |
| Day 10 | 120/180 | 86.1 (13.26) | 79.9 (9.99) | 84.6 (9.04) | 71.1 (10.94) | 74.4 (9.60) | 72.7 (14.17) | 71.3 (13.95) |
| Dialysis | | 60, 104 | 63, 99 | 73, 108 | 58, 94 | 55, 90 | 50, 105 | 49, 92 |
| Day 11 | 180/180 | 78.2 (9.37) | 76.5 (6.47) | 80.7 (8.52) | 79.0 (8.83) | 77.1 (7.07) | 84.4 (10.20) | 74.8 (13.32) |
| | | 61, 91 | 69, 93 | 64, 100 | 65, 94 | 65, 89 | 73, 106 | 52, 98 |
| Day 12 | 180/180 | 84.5 (8.76) | 79.8 (8.96) | 81.4 (9.17) | 71.2 (10.38) | 75.2 (6.64) | 68.9 (10.51) | 68.7 (16.36) |
| Dialysis | 180/240\hat{ } | 70, 101 | 61, 95 | 61, 94 | 53, 89 | 62, 83 | 51, 94 | 34, 94 |
| Day 13 | 180/0 | 78.6 (9.80) | 73.4 (10.40) | 74.2 (9.32) | 74.4 (11.20) | 79.0 (8.90) | 72.1 (10.42) | 71.6 (12.43) |
| | 180/240\hat{ } | 68, 102 | 49, 87 | 58, 88 | 53, 88 | 63, 93 | 56, 87 | 54, 96 |
| Day 14** | 240/240 | 79.5 (6.81) | 75.0 (7.30) | 77.0 (10.10) | 70.0 (10.74) | 73.5 (9.98) | 61.3 (13.15) | 54.5 (8.06) |
| Dialysis | | 74, 88 | 67, 83 | 65, 88 | 56, 81 | 66, 88 | 44, 76 | 48, 66 |
| Day 15** | 240/0 | 72.0 11.78) | 67.0 (9.76) | 73.3 (9.29) | 71.8 (9.88) | 76.0 13.09) | 71.0 10.89) | 69.5 10.28) |
| | | 56, 84 | 54, 77 | 65, 84 | 58, 81 | 66, 95 | 60, 86 | 61, 83 |
| Day 16** | 0 | 77.3 (7.23) | 73.3 (7.23) | 69.7 (3.21) | 75.3 (3.21) | 78.0 (6.24) | 80.3 (9.29) | 75.7 (2.52) |
| | | 69, 82 | 65, 78 | 66, 72 | 73, 79 | 71, 83 | 74, 91 | 73, 78 |
| Day 17** | 0 | 79.4 (10.11) | — | — | — | — | — | — |
| Discharge | | 56, 91 | | | | | | |

\hat{ }Dinner about 10 hours post-am dose.
^{§}Coincides with evening dose administration
*4 out of 14 HD subjects dosed up to 240 mg.
**N = 4 (Cohort 1, Group 4)

TABLE 14
Mean and range of change from baseline in diastolic blood pressure (mmHg) as a function of dose, time and day following oral
administration of nalbuphine HCL ER tablets in hemodialysis patients (N = 14)

| | | DBP Change from baseline (mmHg) mean (SD) and min, max values (N = 14) | | | | | | |
|---|---|---|---|---|---|---|---|---|
| | | in HD patients | | | | | | |
| Day/ | Dose (mg) | PRE AM | POST AM DOSE | | | POST DINNER\hat{ } | | |
| Time | AM/PM | DOSE | 1.5 hr | 4.5 hr | 8 hr | 2 hr^{§} | 6 hr | 10 hr |
| Day 1 | 30/0 | 0 | −4.2 (7.97) | −2.0 (9.55) | 0.7 (8.83) | 2.7 (7.21) | 3.1 (11.61) | −2.3 (9.00) |
| | | | −22, 8 | −31, 9 | −20, 19 | −6, 19 | −11, 42 | −19, 16 |
| Day 2 | 30/30 | 4.0 (8.22) | −2.5 (8.79) | 2.6 (6.15) | 7.4 (10.20) | 6.3 (9.34) | 6.9 (10.55) | 1.3 (7.85) |
| | | −6, 22 | −26, 8 | −7, 14 | −12, 31 | −9, 25 | −11, 31 | −8, 19 |
| Day 3 | 30/30 | 10.6 (6.81) | 6.7 (7.86) | 7.9 (7.27) | −2.9 (11.53) | −3.1 (9.03) | −0.8 (10.56) | −2.5 (6.40) |
| Dialysis | | −4, 25 | −4, 20 | −6, 19 | −34, 15 | −17, 13 | −15, 29 | −12, 10 |
| Day 4 | 30/60 | 2.1 (6.89) | −1.6 (8.35) | 2.5 (9.97) | 0.9 (8.31) | 5.1 (6.38) | 4.6 (12.30) | −0.9 (14.24) |
| | | −9, 11 | −20, 11 | −21, 17 | −15, 14 | −3, 18 | −16, 40 | −14, 40 |
| Day 5 | 60/60 | 6.6 (11.32) | 1.6 (8.11) | 5.5 (8.92) | −3.4 (11.02) | 0.1 (12.88) | −1.1 (8.21) | −4.1 (10.62) |
| Dialysis | | −15, 29 | −12, 16 | −7, 20 | −24, 16 | −18, 33 | −13, 15 | −28, 10 |
| Day 6 | 60/60 | 1.6 (8.95) | −1.7 (6.38) | 1.6 (7.64) | 1.6 (9.65) | 5.4 (7.31) | 3.3 (14.03) | 1.6 (11.76) |
| | | −16, 11 | −16, 5 | −9, 23 | −19, 19 | −4, 23 | −21, 40 | −15, 31 |
| Day 7 | 60/120 | 7.1 (9.75) | 1.1 (9.00) | 6.5 (8.46) | −3.8 (8.66) | −0.9 (10.22) | −1.5 (10.27) | −3.6 (7.15) |
| Dialysis | | −8, 31 | −17, 19 | −12, 19 | −26, 5 | −17, 27 | −16, 22 | −17, 10 |
| Day 8 | 120/120 | 1.8 (9.07) | −4.9 (9.47) | −0.8 (7.85) | −2.2 (8.54) | 2.6 (8.29) | 1.9 (14.33) | 0.4 (10.06) |
| | | −15, 16 | −25, 8 | −12, 11 | −16, 15 | −18, 13 | −18, 32 | −14, 24 |
| Day 9 | 120/120 | 4.9 (9.34) | 3.3 (9.81) | 2.8 (14.42) | 5.5 (6.15) | 9.2 (8.62) | 6.5 (14.32) | 3.2 (12.22) |
| | | −10, 25 | −15, 21 | −26, 23 | −2, 20 | −8, 22 | −11, 44 | −17, 30 |
| Day 10 | 120/180 | 11.7 (11.15) | 5.4 (9.07) | 10.1 (10.26) | −3.3 (9.54) | −0.1 (7.15) | −1.7 (14.21) | −3.1 (11.92) |
| Dialysis | | −2, 37 | −4, 34 | −5, 24 | −25, 10 | −15, 9 | −21, 40 | −22, 27 |
| Day 11 | 180/180 | 3.8 (6.55) | 2.1 (8.90) | 6.3 (8.62) | 4.6 (11.11) | 2.6 (9.10) | 10.0 (12.29) | 0.4 (11.77) |
| | | −8, 20 | −15, 15 | −8, 23 | −19, 30 | −13, 23 | −10, 35 | −18, 33 |
| Day 12 | 180/180 | 10.1 (6.70) | 5.4 (6.40) | 7.0 (9.38) | −3.2 (10.42) | 0.8 (6.95) | −5.5 (8.53) | −5.7 (11.92) |
| Dialysis | 180/240* | −1, 21 | −2, 24 | −13, 26 | −18, 16 | −12, 15 | −21, 10 | −22, 18 |
| Day 13 | 180/0 | 4.1 (11.18) | −1.1 (7.85) | −0.2 (8.85) | −0.1 (11.21) | 4.6 (7.56) | −2.4 (9.94) | −2.8 (8.23) |
| | 240/240* | −14, 24 | −18, 9 | −26, 10 | −31, 15 | −7, 19 | −24, 16 | −14, 12 |
| Day 14** | 240/240 | 12.0 (10.03) | 7.5 (10.41) | 9.5 (16.76) | 2.5 (6.35) | 6.0 (8.12) | −6.3 (9.81) | −13.0 (3.56) |
| Dialysis | | 3, 23 | 1, 23 | −6, 32 | −4, 11 | −2, 16 | −16, 6 | −16, −8 |
| Day 15** | 240/0 | 4.5 (4.80) | −0.5 (1.73) | 5.8 (7.27) | 4.3 (4.50) | 8.5 (7.05) | 3.5 (3.87) | 2.0 (6.78) |
| | | 0, 11 | −2, 1 | −4, 13 | 2, 11 | −1, 16 | −2, 7 | −8, 7 |
| Day 16** | 0 | 13.7 (2.08) | 9.7 (3.06) | 6.0 (7.81) | 11.7 (6.81) | 14.3 (1.15) | 16.7 (10.07) | 12.0 (5.57) |
| | | 12, 16 | 7, 13 | 1, 15 | 4, 17 | 13, 15 | 6, 26 | 6, 17 |
| Day 17** | 0 | 5.3 (8.77) | — | — | — | — | — | — |
| Discharge | | −9, 17 | | | | | | |

\hat{ }Dinner about 10 hours post-am dose.
^{§}Coincides with evening dose adminisfration
*4 out of 14 HD subjects dosed up to 240 mg.
**N = 4 (Cohort 1, Group 4)

TABLE 15
Mean and range of systolic blood pressure (mmHg) as a function of dose, time and day following oral administration of
nalbuphine HCL ER tablets in hemodialysis patients (N = 14)

| | | SBP (mmHg) mean (SD) and min, max values (N = 14) in HD patients | | | | | | |
|---|---|---|---|---|---|---|---|---|
| Day/ | Dose (mg) | PRE AM | POST AM DOSE | | | POST DINNER | | |
| Time | AM/PM | DOSE | 1.5 hr | 4.5 hr | 8 hr | 2 hr^{§} | 6 hr | 10 hr |
| Day−1 | 0 | 139.6 (20.79) | 136.3 (20.32) | 151.0 (24.32) | 130.8 (25.36) | 117.0 (19.54) | 118.0 (22.11) | 115.8 (30.58) |
| Dialysis | | 119, 179 | 114, 161 | 129, 174 | 94, 151 | 98, 143 | 97, 148 | 74, 145 |
| Day 1 | 30/0 | 130.2 (18.53) | 121.3 (18.64) | 123.8 (19.16) | 128.8 (21.22) | 134.9 (18.28) | 137.8 (21.62) | 134.6 (19.10) |
| | | 88, 158 | 92, 164 | 96, 155 | 96, 155 | 91, 154 | 99, 183 | 99, 167 |
| Day 2 | 30/30 | 137.5 (21.20) | 127.0 (16.79) | 136.2 (14.90) | 140.8 (18.30) | 143.0 (18.35) | 148.3 (14.19) | 143.1 (21.90) |
| | | 96, 180 | 95, 151 | 109, 167 | 107, 163 | 109, 176 | 119, 173 | 101, 181 |
| Day 3 | 30/30 | 151.2 (21.23) | 144.2 (15.32) | 146.1 (13.62) | 130.7 (20.52) | 127.3 (21.82) | 138.2 (25.79) | 128.0 (20.75) |
| Dialysis | | 106, 184 | 120, 181 | 127, 179 | 84, 164 | 84, 157 | 82, 174 | 90, 161 |
| Day 4 | 30/60 | 130.5 (16.56) | 128.8 (18.99) | 132.5 (20.65) | 131.9 (23.78) | 142.9 (22.23) | 142.2 (22.97) | 136.0 (27.66) |
| | | 97, 167 | 94, 169 | 95, 165 | 78, 167 | 101, 175 | 101, 188 | 81, 175 |
| Day 5 | 60/60 | 142.6 (16.07) | 137.1 (12.23) | 145.4 (20.79) | 127.0 (14.77) | 130.9 (23.43) | 137.5 (18.71) | 126.8 (21.69) |
| Dialysis | | 115, 168 | 111, 158 | 116, 180 | 103, 151 | 83, 178 | 109, 173 | 93, 164 |
| Day 6 | 60/60 | 130.6 (21.99) | 131.4 (20.27) | 139.4 (17.70) | 130.9 (18.73) | 144.0 (23.25) | 139.2 (24.91) | 137.2 (21.78) |
| | | 92, 164 | 102, 168 | 112, 168 | 101, 157 | 111, 180 | 91, 186 | 105, 169 |
| Day 7 | 60/120 | 145.9 (21.67) | 141.0 (19.03) | 145.4 (17.18) | 130.2 (16.73) | 131.8 (21.97) | 132.9 (21.66) | 130.8 (20.49) |
| Dialysis | | 115, 176 | 113, 166 | 113, 173 | 106, 163 | 97, 159 | 95, 163 | 102, 160 |
| Day 8 | 120/120 | 128.8 (17.62) | 125.6 (22.15) | 128.9 (18.50) | 134.9 (21.85) | 142.0 (23.32) | 143.1 (23.83) | 135.4 (27.92) |
| | | 98, 158 | 98, 160 | 96, 163 | 91, 163 | 95, 167 | 101, 182 | 94, 178 |
| Day 9 | 120/120 | 147.6 (21.82) | 137.8 (17.89) | 140.4 (14.92) | 151.0 (20.81) | 150.9 (17.65) | 148.6 (27.34) | 144.9 (27.38) |
| | | 115, 180 | 105, 166 | 114, 162 | 114, 184 | 124, 185 | 95, 199 | 104, 189 |
| Day 10 | 120/180 | 157.6 (22.16) | 142.8 (18.82) | 150.8 (19.37) | 136.7 (23.42) | 136.8 (26.04) | 132.9 (26.60) | 131.9 (23.91) |
| Dialysis | | 111, 190 | 107, 166 | 115, 190 | 101, 177 | 87, 172 | 80, 184 | 86, 165 |
| Day 11 | 180/180 | 141.6 (23.51) | 135.4 (17.19) | 139.8 (17.15) | 141.6 (20.35) | 142.9 (17.23) | 150.9 (21.81) | 137.0 (20.05) |
| | | 88, 166 | 108, 164 | 99, 162 | 104, 167 | 118, 176 | 116, 189 | 104, 172 |
| Day 12 | 180/180 | 148.6 (19.75) | 140.6 (21.07) | 147.1 (21.79) | 134.9 (23.33) | 136.6 (25.20) | 126.1 (22.44) | 127.4 (28.04) |
| Dialysis | 180/240* | 118, 176 | 104, 180 | 95, 178 | 98, 179 | 97, 178 | 89, 153 | 70, 162 |
| Day 13 | 180/0 | 140.4 (21.35) | 131.1 (17.24) | 132.7 (19.72) | 139.7 (23.40) | 146.6 (26.13) | 138.5 (22.88) | 132.9 (29.27) |
| | 180/240* | 103, 173 | 97, 156 | 100, 156 | 103, 176 | 97, 196 | 109, 182 | 70, 166 |
| Day | 240/240 | 138.8 (16.98) | 138.0 (12.33) | 144.0 (7.44) | 122.5 (19.12) | 126.0 (31.95) | 111.0 (20.12) | 106.0 (19.58) |
| 14** | | 117, 158 | 122, 152 | 134, 151 | 99, 140 | 102, 172 | 81, 123 | 92, 135 |
| Dialysis | | | | | | | | |
| Day | 240/0 | 133.0 (8.12) | 109.5 (13.28) | 132.3 (25.71) | 126.3 (19.97) | 134.5 (36.24) | 123.3 (28.11) | 124.5 (17.62) |
| 15** | | 127, 145 | 97, 128 | 115, 170 | 113, 156 | 99, 185 | 92, 158 | 110, 148 |
| Day | 0 | 137.7 (13.65) | 125.0 (10.39) | 127.3 (1.53) | 132.3 (12.22) | 128.0 (12.77) | 142.3 (2.08) | 135.3 (12.10) |
| 16** | | 123, 150 | 119, 137 | 126, 129 | 119, 143 | 114, 139 | 140, 144 | 126, 149 |
| Day | 0 | 147.5 (18.38) | | | | | | |
| 17** | | 105, 171 | | | | | | |
| Dialysis | | | | | | | | |

\hat{ } Dinner about 10 hours post-am dose.
^{§}Coincides with evening dose adminisfration
*4 out of 14 HD subjects dosed up to 240 mg.
**N = 4

TABLE 16
Mean and range of change from baseline in systolic blood pressure (mmHg) as a function of dose, time and day following oral
administration of nalbuphine HCL ER tablets in hemodialysis patients (N = 14)

| | | SBP Change from baseline (mmHg) mean (SD) and min, max values (N = 14) | | | | | | |
|---|---|---|---|---|---|---|---|---|
| | | in HD patients | | | | | | |
| Day/ | Dose (mg) | PRE AM | POST AM DOSE | | | POST DINNER\hat{ } | | |
| Time | AM/PM | DOSE | 1.5 hr | 4.5 hr | 8 hr | 2 hr^{§} | 6 hr | 10 hr |
| Day 1 | 30/0 | 0 | −8.9 (11.75) | −6.4 (15.91) | −1.4 (16.29) | 4.7 (13.08) | 7.6 (18.83) | 4.4 (19.57) |
| | | 0 | −35, 10 | −41, 17 | −36, 29 | −15, 30 | −22, 55 | −32, 39 |
| Day 2 | 30/30 | 7.3 (12.19) | −3.2 (17.23) | 6.0 (15.33) | 10.6 (16.35) | 12.8 (16.74) | 18.1 (15.00) | 12.9 (16.34) |
| | | −18, 23 | −36, 27 | −25, 37 | −15, 34 | −10, 48 | −5, 45 | −17, 47 |
| Day 3 | 30/30 | 21.0 (14.68) | 14.0 (15.49) | 15.9 (16.40) | 0.5 (15.56) | −2.9 (12.99) | 8.0 (17.20) | 0.0 (13.64) |
| Dialysis | | −4, 50 | −10, 42 | −15, 39 | −50, 17 | −29, 18 | −20, 40 | −19, 35 |
| Day 4 | 30/60 | 2.1 (9.90) | 0.4 (17.84) | 4.1 (14.90) | 3.5 (11.71) | 14.5 (12.35) | 13.8 (20.41) | 7.6 (22.43) |
| | | −18, 13 | −30, 30 | −24, 31 | −14, 20 | −5, 34 | −13, 60 | −38, 47 |
| Day 5 | 60/60 | 14.1 (15.57) | 8.7 (19.67) | 16.9 (21.14) | −1.4 (17.00) | 2.4 (18.61) | 9.1 (14.19) | −1.6 (17.30) |
| Dialysis | | −18, 47 | −26, 52 | −20, 55 | −31, 34 | −31, 50 | −13, 34 | −33, 32 |
| Day 6 | 60/60 | 2.1 (21.59) | 2.9 (16.50) | 10.9 (19.91) | 2.5 (16.44) | 15.6 (18.78) | 10.8 (17.34) | 8.8 (22.60) |
| | | −50, 30 | −23, 44 | −15, 53 | −19, 28 | −9, 56 | −7, 58 | −45, 42 |
| Day 7 | 60/120 | 17.5 (18.86) | 12.6 (20.64) | 17.0 (17.85) | 1.8 (15.59) | 3.4 (17.05) | 4.5 (17.46) | 2.4 (18.95) |
| Dialysis | | −27, 42 | −32, 53 | −24, 41 | −24, 22 | −22, 35 | −21, 31 | −46, 26 |
| Day 8 | 120/120 | 2.6 (18.10) | −2.8 (22.72) | 0.4 (19.71) | 6.4 (16.66) | 13.6 (14.79) | 14.7 (21.09) | 6.9 (23.61) |
| | | −35, 34 | −39, 36 | −27, 40 | −23, 36 | −18, 34 | −18, 54 | −51, 44 |
| Day 9 | 120/120 | 19.1 (18.39) | 9.4 (16.15) | 12.0 (21.99) | 22.6 (21.34) | 22.5 (16.18) | 20.1 (24.83) | 16.5 (25.18) |
| | | −11, 42 | −13, 32 | −29, 46 | −1, 68 | 4, 51 | −19, 71 | −33, 50 |
| Day 10 | 120/180 | 29.1 (18.47) | 14.4 (14.10) | 22.4 (17.98) | 8.3 (17.29) | 8.4 (19.15) | 4.4 (19.11) | 3.5 (14.73) |
| Dialysis | | −3, 62 | −2, 38 | −3, 52 | −27, 35 | −27, 48 | −15, 56 | −15, 31 |
| Day 11 | 180/180 | 13.1 (12.54) | 6.9 (19.11) | 11.4 (12.82) | 13.1 (21.60) | 14.4 (19.12) | 22.5 (19.68) | 8.6 (17.73) |
| | | −11, 35 | −20, 51 | −8, 36 | −34, 52 | −9, 52 | −3, 55 | −20, 44 |
| Day 12 | 180/180 | 20.2 (15.15) | 12.1 (17.20) | 18.6 (16.08) | 6.5 (19.08) | 8.1 (19.67) | −2.3 (19.35) | −1.1 (17.01) |
| Dialysis | | −5, 52 | −11, 46 | −2, 52 | −28, 34 | −35, 35 | −42, 26 | −23, 28 |
| Day 13 | 180/0 | 11.9 (15.91) | 2.7 (13.59) | 4.3 (16.39) | 11.3 (22.83) | 18.2 (18.51) | 10.1 (19.99) | 4.5 (21.10) |
| | 180/240* | −12, 49 | −21, 32 | −39, 31 | −35, 48 | −11, 53 | −26, 43 | −22, 32 |
| Day 14** | 240/240* | 23.5 (23.95) | 22.8 (21.30) | 28.8 (27.43) | 7.3 (16.50) | 10.8 (16.96) | −4.3 (12.69) | −9.3 (14.66) |
| Dialysis | | −2, 55 | 3, 50 | 0, 63 | −7, 29 | −12, 29 | −21, 7 | −21, 11 |
| Day 15** | 240/0 | 17.8 (15.78) | −5.8 (10.37) | 17.0 (13.69) | 11.0 (10.95) | 19.3 (15.20) | 8.0 (12.03) | 9.3 (12.97) |
| | | 2, 39 | −15, 9 | −2, 27 | −1, 25 | 11, 42 | −7, 20 | −7, 22 |
| Day 16** | 0 | 31.7 (3.06) | 19.0 (16.64) | 21.3 (17.62) | 26.3 (24.01) | 22.0 (8.72) | 36.3 (14.01) | 29.3 (19.50) |
| | | 29, 35 | 0, 31 | 7, 41 | 0, 47 | 12, 28 | 25, 52 | 7, 43 |
| Day 17** | 0 | 20.2 (17.06) | | | | | | |
| Dialysis | | −9, 45 | | | | | | |

\hat{ }Dinner about 10 hours post-am dose.
^{§}Coincides with evening dose administration

TABLE 17
Mean and range of heart rate (HR) as a function of dose, time and day following oral
administration of nalbuphine HCL ER tablets in hemodialysis patients (N = 14)

| | | HR (beats/min) mean (SD) and min, max values (N = 14) in HD patients | | | | |
|---|---|---|---|---|---|---|
| | | | | | | POST |
| Day/ | Dose (mg) | PRE AM | POST AM DOSE | | | DINNER\hat{ } |
| Time | AM/PM | DOSE | 1.5 hr | 4.5 hr | 8 hr | 2 hr^{§} |
| Day−1 | 0 | 78.5 (13.25) | 73.8 (10.69) | 79.8 (9.46) | 93.0 (25.53) | 78.5 (16.09) |
| Dialysis | | 54, 102 | 65, 89 | 66, 86 | 70, 126 | 58, 97 |
| Day 1 | 30/0 | 75.1 (11.45) | 73.9 (12.63) | 69.5 (9.25) | 70.8 (9.88) | 74.4 (8.91) |
| | | 52, 88 | 54, 95 | 50, 84 | 54, 86 | 57, 85 |
| Day 2 | 30/30 | 71.4 (10.27) | 71.2 (11.99) | 68.8 (11.38) | 71.2 (11.96) | 73.5 (9.72) |
| | | 50, 89 | 48, 88 | 49, 86 | 48, 88 | 56, 87 |
| Day 3 | 30/30 | 70.3 (9.24) | 69.1 (10.23) | 70.7 (10.61) | 74.5 (11.99) | 77.1 (13.39) |
| Dialysis | | 48, 82 | 49, 88 | 49, 84 | 53, 100 | 56, 99 |
| Day 4 | 30/60 | 73.0 (11.34) | 72.1 (11.86) | 72.0 (11.65) | 72.6 (10.94) | 73.2 (10.02) |
| | | 52, 88 | 53, 91 | 53, 89 | 54, 89 | 54, 87 |
| Day 5 | 60/60 | 71.7 (12.40) | 69.4 (11.39) | 71.0 (9.91) | 74.7 (14.53) | 78.1 (13.20) |
| Dialysis | | 49, 91 | 46, 84 | 55, 87 | 54, 104 | 59, 101 |
| Day 6 | 60/60 | 75.8 (13.03) | 72.7 (13.10) | 71.1 (9.98) | 71.1 (11.06) | 72.6 (11.67) |
| | | 54, 94 | 52, 97 | 55, 86 | 52, 85 | 54, 94 |
| Day 7 | 60/120 | 72.8 (12.19) | 68.8 (11.73) | 72.9 (14.69) | 76.6 (12.97) | 76.1 (12.70) |
| Dialysis | | 48, 87 | 47, 84 | 50, 105 | 56, 96 | 57, 96 |
| Day 8 | 120/120 | 74.2 (14.07) | 72.7 (13.06) | 69.7 (11.81) | 73.1 (11.82) | 77.0 (13.50) |
| | | 51, 93 | 49, 90 | 50, 89 | 53, 91 | 59, 102 |
| Day 9 | 120/120 | 74.9 (13.61) | 72.1 (11.89) | 70.1 (11.65) | 70.6 (9.48) | 73.1 (10.41) |
| | | 51, 98 | 51, 89 | 51, 90 | 57, 86 | 56, 89 |
| Day 10 | 120/180 | 73.6 (12.18) | 69.3 (11.78) | 74.1 (11.91) | 76.2 (11.36) | 79.6 (12.55) |
| Dialysis | | 52, 95 | 50, 90 | 56, 94 | 60, 96 | 61, 96 |
| Day 11 | 180/180 | 74.6 (12.85) | 71.6 (11.65) | 70.6 (11.17) | 70.5 (11.35) | 73.8 (11.85) |
| | | 57, 96 | 57, 90 | 54, 88 | 52, 88 | 59, 100 |
| Day 12 | 180/180 | 71.5 (12.38) | 70.1 (11.03) | 72.0 (10.34) | 76.6 (12.41) | 75.9 (11.32) |
| Dialysis | 180/240* | 57, 100 | 52, 86 | 56, 88 | 57, 100 | 60, 93 |
| Day 13 | 180/0 | 74.6 (11.05) | 71.9 (10.26) | 71.1 (10.33) | 73.1 (10.86) | 75.0 (10.38) |
| | 240/240* | 57, 97 | 57, 90 | 57, 88 | 56, 96 | 59, 96 |
| Day 14** | 240/240 | 74.5 (8.43) | 68.0 (7.83) | 74.5 (11.56) | 77.8 (10.28) | 86.5 (11.90) |
| Dialysis | | 62, 80 | 60, 78 | 64, 91 | 66, 91 | 70, 97 |
| Day 15** | 240/0 | 78.8 (11.79) | 77.3 (13.33) | 78.0 (0.82) | 78.8 (8.85) | 84.3 (8.77) |
| | | 62, 87 | 59, 89 | 77, 79 | 66, 86 | 73, 94 |
| Day 16** | 0 | 77.3 (4.16) | 79.7 (3.21) | 75.3 (5.03) | 84.0 (2.65) | 83.0 (6.00) |
| | | 74, 82 | 76, 82 | 70, 80 | 82, 87 | 77, 89 |
| Day 17** | 0 | 70.9 (9.73)56, | | | | |
| Dialysis | | 88 | | | | |

\hat{ }Dinner about 10 hours post-am dose.
^{§}Coincides with evening dose administration.
*4 out of 14 HD subjects dosed up to 240 mg.
**N = 4

TABLE 18
Mean and range of change in heart rate as a function of dose, time and day following
oral administration of nalbuphine HCL ER tablets in hemodialysis patients (N = 14)

| | | HR change from baseline (beats/min) | | | | |
|---|---|---|---|---|---|---|
| | | mean (SD) and min, max values (N = 14) in HD patients | | | | |
| | | | | | | POST |
| Day/ | Dose (mg) | PRE AM | POST AM DOSE | | | DINNER\hat{ } |
| Time | AM/PM | DOSE | 1.5 hr | 4.5 hr | 8 hr | 2 hr^{§} |
| Day 1 | 30/0 | 0 | −1.3 (7.87) | −5.6 (5.28) | −4.3 (5.97) | −0.7 (5.43) |
| | | 0 | −14, 16 | −15, 2 | −19, 3 | −11, 10 |
| Day 2 | 30/30 | −3.7 (7.49) | −3.9 (7.96) | −6.3 (7.73) | −3.9 (10.17) | −1.7 (6.95) |
| | | −15, 9 | −23, 10 | −26, 6 | −24, 9 | −11, 10 |
| Day 3 | 30/30 | −4.9 (6.71) | −6.0 (9.86) | −4.4 (7.92) | −0.6 (10.87) | 2.0 (10.09) |
| Dialysis | | −16, 6 | −22, 14 | −20, 8 | −22, 18 | −19, 23 |
| Day 4 | 30/60 | −2.6 (7.56) | −3.6 (8.59) | −3.6 (9.15) | −3.0 (10.20) | −2.4 (8.01) |
| | | −17, 6 | −22, 10 | −21, 9 | −22, 15 | −19, 9 |
| Day 5 | 60/60 | −3.9 (10.27) | −6.3 (8.90) | −4.6 (11.11) | −0.9 (13.09) | 2.5 (10.74) |
| Dialysis | | −20, 11 | −24, 10 | −26, 11 | −21, 23 | −16, 20 |
| Day 6 | 60/60 | 0.1 (11.17) | −2.9 (10.56) | −4.5 (8.47) | −4.5 (10.11) | −3.1 (9.77) |
| | | −18, 24 | −22, 17 | −22, 6 | −22, 15 | −23, 7 |
| Day 7 | 60/120 | −2.9 (10.17) | −6.9 (11.11) | −2.7 (12.97) | 1.0 (10.67) | 0.5 (11.31) |
| Dialysis | | −22, 14 | −26, 8 | −24, 24 | −17, 15 | −17, 17 |
| Day 8 | 120/120 | −1.5 (10.55) | −2.9 (10.54) | −5.9 (10.25) | −2.5 (11.22) | 1.4 (13.52) |
| | | −20, 15 | −21, 12 | −21, 10 | −22, 12 | −23, 20 |
| Day 9 | 120/120 | −0.7 (15.83) | −3.5 (11.13) | −5.6 (10.01) | −5.0 (10.30) | −2.5 (10.29) |
| | | −22, 33 | −22, 15 | −24, 13 | −24, 12 | −21, 15 |
| Day 10 | 120/180 | −2.1 (11.72) | −6.4 (12.25) | −1.6 (11.33) | 0.6 (12.95) | 3.9 (11.91) |
| Dialysis | | −21, 21 | −31, 16 | −21, 16 | −21, 23 | −20, 21 |
| Day 11 | 180/180 | −1.0 (11.26) | −4.0 (11.01) | −5.0 (9.71) | −5.1 (10.83) | −1.9 (11.61) |
| | | −19, 17 | −23, 16 | −25, 10 | −22, 14 | −21, 12 |
| Day 12 | 180/180 | −4.1 (13.81) | −5.6 (11.63) | −3.6 (12.11) | 1.0 (12.56) | 0.3 (11.52) |
| Dialysis | 180/240* | −26, 26 | −29, 12 | −25, 14 | −21, 19 | −20, 17 |
| Day 13 | 180/0 | −1.0 (11.45) | −3.7 (11.05) | −4.6 (12.24) | −2.6 (12.28) | −0.6 (12.78) |
| | 240/240* | −16, 23 | −22, 11 | −24, 12 | −25, 14 | −22, 17 |
| Day 14** | 240/240* | 0.3 (7.68) | −6.3 (8.18) | 0.3 (7.04) | 3.5 (9.15) | 12.3 (11.84) |
| Dialysis | | −8, 9 | −11, 6 | −10, 6 | −10, 10 | −2, 27 |
| Day 15** | 240/0 | 4.5 (4.20) | 3.0 (2.45) | 3.8 (13.60) | 4.5 (7.23) | 10.0 (7.16) |
| | | −1, 9 | 1, 6 | −11, 20 | −2, 13 | 2, 17 |
| Day 16** | 0 | −2.3 (10.41) | 0.0 (10.44) | −4.3 (4.04) | 4.3 (6.81) | 3.3 (3.21) |
| | | −14, 6 | −7, 12 | −8, 0 | −1, 12 | 1, 7 |
| Day 17** | 0 | −6.1 (13.03) | — | — | — | — |
| Dialysis | | −27, 14 | | | | |

\hat{ }Dinner about 10 hours post-am dose.
^{§}Coincides with evening dose administration
*4 out of 14 HD subjects dosed up to 240 mg.
**N = 4

The embodiments described herein and illustrated by the foregoing examples should be understood to be illustrative of the present invention, and should not be construed as limiting. On the contrary, the present disclosure embraces alternatives and equivalents thereof, as embodied by the appended claims. Each reference disclosed herein is incorporated by reference herein in its entirety.

1. A method of treating pruritus in a subject in need of such treatment, comprising administering an effective amount of an anti-pruritic agent to the subject, wherein the anti-pruritic agent is nalbuphine or a pharmaceutically acceptable salt, solvate or ester thereof, and the method provides a therapeutic effect without producing a substantial aquaretic effect.

2. The method of claim 1, which does not significantly alter the total daily urine volume in the subject upon administration of the anti-pruritic agent.

3. The method of claim 2, wherein the subject has a total daily urine volume of no more than about 9500 ml upon administration of the anti-pruritic agent.

4. (canceled)

5. (canceled)

6. The method of claim 3, wherein the subject has a total daily urine volume of about 500 ml to about 9500 ml upon administration of the anti-pruritic agent.

7. (canceled)

8. (canceled)

9. (canceled)

10. The method of claim 1, which does not significantly alter urine specific gravity in the subject upon administration.

11. The method of claim 10, wherein the urine specific gravity in the subject ranges from about 0.900 to about 1.133 upon administration of the anti-pruritic agent.

12. (canceled)

13. (canceled)

14. The method of claim 1, which does not significantly alter the urobilinogen content of urine in the subject upon administration of the anti-pruritic agent.

15. The method of claim 14, wherein the urobilinogen content of urine in the subject ranges from about 0 to about 21 μmol/L upon administration of the anti-pruritic agent.

16. (canceled)

17. (canceled)

18. The method of claim 1, which does not significantly alter the pH of urine in the subject upon administration.

19. The method of claim 18, wherein the pH of urine in the subject ranges from about 4.5 to about 8.8 upon administration of the anti-pruritic agent.

20. (canceled)

21. (canceled)

22. The method of claim 1, which does not significantly alter the serum sodium content of the subject upon administration of the anti-pruritic agent.

23. The method of claim 1, which does not induce hypernatremia in the subject upon administration.

24. The method of claim 22, wherein the serum sodium content of the subject ranges from about 122 to about 161 mmol/L upon administration of the anti-pruritic agent.

25. (canceled)

26. (canceled)

27. The method of claim 23, wherein the serum sodium content of the subject is no more than about 161 mmol/L after administration of the anti-pruritic agent.

28. (canceled)

29. (canceled)

30. The method of claim 1, which does not significantly alter the heart rate of the subject upon administration of the anti-pruritic agent.

31. The method of claim 30, wherein the hear rate of the subject ranges from about 45 to about 113 beats/min upon administration of the anti-pruritic agent, provided that the subject does not need hemodialysis.

32. (canceled)

33. (canceled)

34. The method of claim 30, wherein the heart rate of the subject is no more than about 112 beats/min upon administration of the anti-pruritic agent, provided that the subject does not need hemodialysis.

35. (canceled)

36. (canceled)

37. The method of claim 30, wherein the heart rate of the subject ranges from about 41 to about 140 beats/min upon administration of the anti-pruritic agent, provided that the subject needs hemodialysis.

38. (canceled)

39. (canceled)

40. The method of claim 30, wherein the heart rate of the subject is no more than about 139 beats/min upon administration of the anti-pruritic agent, provided that the subject needs hemodialysis.

41. (canceled)

42. (canceled)

43. The method of claim 1, which does not significantly alter blood pressure of the subject upon administration of the anti-pruritic agent.

44. The method of claim 43, which does not induce hypotension in the subject upon administration of the anti-pruritic agent.

45. The method of claim 44, wherein the systolic blood pressure ranges from about 70 to about 163 mmHg upon administration of the anti-pruritic agent in a subject that does not need hemodialysis.

46. (canceled)

47. (canceled)

48. The method of claim 44, wherein the systolic blood pressure ranges from about 63 to about 220 mmHg upon administration of the anti-pruritic agent in a subject that needs hemodialysis.

49. (canceled)

50. (canceled)

51. The method of claim 44, wherein the diastolic blood pressure ranges from about 37 to about 103 mmHg upon administration of the anti-pruritic agent in a subject that does not need hemodialysis.

52. (canceled)

53. (canceled)

54. The method of claim 44, wherein the diastolic blood pressure ranges from about 30 to about 120 mmHg upon administration of the anti-pruritic agent in a subject that needs hemodialysis.

55. (canceled)

56. (canceled)

57. The method of claim 1, wherein said subject is suffering from a pruritic condition, and said pruritic condition comprises atopic dermatitis, nervous dermatitis, contact dermatitis, seborrheic dermatitis, autosensitization dermatitis, caterpillar dermatitis, asteatosis, senile pruritus cutaneous, insect sting, photosensitive dermatosis, urticaria, prurigo, herpes, impetigo, eczema, tinea, lichen, psoriasis, scabies and acne vulgaris, or visceral diseases complicated with pruritus.

58. The method of claim 57, wherein said visceral diseases complicated with pruritus comprise malignant tumors, diabetes mellitus, hepatic diseases, renal failure, or pregnancy.

59. The method of claim 1, wherein said subject is suffering from a skin change comprising pruritus secondary to inflamed skin, pruritus arising from conditions of non-diseased skin, pruritus associated with chronic secondary scratch, or skin lesions resulting from an underlying medical condition.

60. The method of claim 59, wherein said underlying medical condition has an origin comprising dermatologic origin, systemic disease origin, neurologic origin, psychogenic origin, or mixed origin.

61. The method of claim 1, wherein said subject has uremic pruritus or prurigo nodularis.

62. The method of claim 1, wherein the anti-pruritus agent is administered at an initial oral dose of from about 15 mg to about 30 mg twice a day and then titrated to an effective dose.

63. The method of claim 1, wherein the anti-pruritic agent is administered at an initial dose of from about 15 mg to about 30 mg once a day and then titrated to an effective dose.

64. The method of claim 1, wherein the anti-pruritic agent is administered at an initial dose of from about 15 mg to about 30 mg twice a day or once a day for about 2 to 3 days and then titrated to an effective dose at about 15 mg to about 30 mg increment.

65. The method of claim 1, wherein the maximum dose of the anti-pruritic agent is about 480 mg when said agent is administered to a subject twice a day or about 240 mg when said agent is administered to a subject once a day.

66. The method of claim 1, wherein the anti-pruritic agent is administered with an AM dosage and a PM dosage and wherein the PM dosage is higher than the AM dosage, or vice versa.

67. The method of claim 1, wherein the anti-pruritic agent is administered at a dose of about 60 mg or about 120 mg twice a day to a subject with uremic pruritus or renal impairment or about 90 mg or about 180 mg twice a day to a subject without a renal impairment condition.

68. The method of claim 1, wherein the anti-pruritic agent is in an extended release oral dosage form.

69. The method of claim 68, wherein the anti-pruritic agent is administered in a formulation comprising nalbuphine hydrochloride, mannitol, hydroxypropyl cellulose, locust bean gum, xanthan gum, calcium sulfate dihydrate, and magnesium stearate.
